(19) United States
(12) Patent Application Publication
(10) Pub. No.: US 2026/0130924 A1
(43) Pub. Date: May 14, 2026

(54) COMPOSITIONS AND METHODS OF TREATING SUBJECTS WITH BRCA1 MUTATION OR DEFICIENCY

(71) Applicant: BOARD OF REGENTS, THE UNIVERSITY OF TEXAS SYSTEM, Austin, TX (US)

(72) Inventors: Boyi GAN, Houston, TX (US); Guang LEI, Houston, TX (US)

(21) Appl. No.: 19/118,723

(22) PCT Filed: Oct. 6, 2023
(86) PCT No.: PCT/US2023/076276

§ 371 Date: Apr. 4, 2025

Related U.S. Application Data
(60) Provisional application No. 63/378,651, filed on Oct. 6, 2022.

(51) Int. Cl. A61K 31/585; A61K 31/381; A61K 31/4184; A61K 31/437; A61K 31/454; A61K 31/496; A61K 31/502; A61K 31/5025; A61K 31/55; A61K 45/06; A61P 35/00

(52) U.S. Cl. CPC A61K 31/585; A61K 31/381; A61K 31/4184; A61K 31/437; A61K 31/454; A61K 31/496; A61K 31/502; A61K 31/5025; A61K 31/55; A61K 45/06; A61P 35/00

(57) ABSTRACT

The present disclosure provides methods for treating cancer comprising administering to a subject in need thereof a therapeutically effective amount of a GPX4 inhibitor and a PARP inhibitor, wherein the subject is BRCA1 deficient (e.g., carries a BRCA1 mutation). The present disclosure also provides methods of selecting a subject afflicted with a cancer as suitable for treatment with a GPX4 inhibitor and a PARP inhibitor comprising identifying the subject as having a BRCA1 deficiency and treating the subject with a GPX4 inhibitor and a PARP inhibitor.

### CROSS REFERENCE TO RELATED APPLICATIONS

This patent application claims the benefit of U.S. Provisional Patent Application Ser. No. 63/378,651, filed Oct. 6, 2022 which is incorporated herein by reference in its entirety for all purposes.

### GOVERNMENTAL RIGHTS

This invention was made with government support under grant number CA181196 awarded by the National Institutes of Health. The government has certain rights in the invention.

### FIELD OF THE DISCLOSURE

The present disclosure relates to methods and therapeutic compositions for the treatment of various types of cancers in subjects having a BRCA1 deficiency (e.g., a BRCA1 mutation). The compositions and methods relate to administration of a GPX4 inhibitor and a PARP inhibitor in such subjects.

### BACKGROUND

Ferroptosis is a form of non-apoptotic cell death induced by excessive lipid peroxidation (Dixon, S. J., et al., Cell 149, 1060-1072 (2012); Stockwell, B. R., et al., Cell 171, 273-285 (2017)). Incorporation of polyunsaturated fatty acids (PUFAs; fatty acids that contain more than one double bond) into phospholipids (PLs) in cellular membranes is critical for cellular functions. However, chemical features in PUFAs also render PUFA-PLs particularly susceptible to lipid peroxidation in iron- and oxygen-rich cellular environments. If left unchecked, these toxic lipid peroxides can damage membrane integrity and induce ferroptotic cell death (Conrad, M., et al., Nat. Chem Bio. 15, 1137-47 (2019)).

Cells have evolved at least two defense mechanisms to suppress ferroptosis. In the first, glutathione peroxidase 4 (GPX4) utilizes reduced glutathione (GSH) to detoxify lipid hydroperoxides and inhibit ferroptosis (Jiang, L., et al., Nature 520, 57-62 (2015); Zhang, Y., et al., Nat Cell Biol 20, 1181-1192 (2018)). Many cancer cells rely on solute carrier family 7 member 11 (SLC7A11)-mediated cystine transport to obtain cysteine for GSH biosynthesis (Yang, W. S., et al., Cell 156, 317-331 (2014)). The SLC7A11-GPX4 signaling axis represents the major cellular defense system against ferroptosis, and inactivation of GPX4 or SLC7A11 by corresponding ferroptosis inducers induces ferroptosis in many cancer cells (Dixon, S. J., et al., Cell 149, 1060-1072 (2012) Jiang, L., et al., Nature 520, 57-62 (2015); Zhang, Y., et al., Nat Cell Biol 20, 1181-1192 (2018)). In the second, ferroptosis suppressor protein 1 (FSP1; also called AIFM2) acts as another ferroptosis inhibitor in parallel to GPX4 to suppress ferroptosis. Mechanistically, FSP1 functions as an oxidoreductase primarily localized on the plasma membrane to reduce ubiquinone (CoQ) to ubiquinol (CoQH2), which then acts as a lipophilic radical trapping antioxidant (RTA) to detoxify lipid hydroperoxides (Friedmann Angeli, J. P., et al., Nat Cell Biol 16, 1180-1191 (2014); Bersuker, K., et al., Nature 575, 688-692 (2019)). In addition, mitochondrial enzyme dihydroorotate dehydrogenase (DHODH) operates independently of GPX4 or FSP1 to suppress ferroptosis in the mitochondrial inner membrane by reducing CoQ to CoQH2 (Feng, C. et al., J Virol., 95 (17): e0026421 (2021)). Ferroptosis has recently emerged as a critical tumor suppression mechanism (Dixon, S. J., et al., Cell 149, 1060-1072 (2012); Stockwell, B. R., et al., Cell 171, 273-285 (2017); Doll, S., et al., Nature 575, 693-698 (2019); Koppula, P., Zhuang, L. & Gan, B., Protein Cell (2020)). However, mechanisms underlying ferroptosis vulnerabilities under specific cancer or genetic contexts remain unexplored.

Breast cancer-associated gene 1 (BRCA1) is a tumor suppressor gene encoding a large protein that is involved in many essential biological processes, including DNA damage repair, cell cycle checkpoints, chromatin remodeling, transcriptional regulation, and protein ubiquitination (Liu, J., Nat. Comm. 11 (2020)), The deficiency of BRCA1 induces severe genome instability, eventually leading to tumorigenesis (Deng, C. X., Nucleic Acids Res. 34, 1416-26 (2006); Gorodeska, I., J. Cancer 10, 2109-27 (2019). Mutations in BRCA1 predispose carriers to pancreatic, prostate, colorectal, and notably, breast and ovarian cancers (Silver, D. P., Livingtston, D. M., Cancer Discov. 2, 679-84 (2012); Gorodetska, I., et al., J. Cancer, 10, 2109-27 (2019); Mersch, J., et al., Cancer, 121, 269-75 (2015); Cavanagh, H., Rogers, K. M., Hered. Cancer Clin. Pract., 13 (2015); Roy R., et al, Nat. Rev. Cancer, 12, 68-78 (20211)).

Inhibitors of poly(ADP-ribose) polymerase (PARP inhibitor, such as olaparib and niraparib) have been used clinically to treat BRCA1-deficient cancers (Ison G., et al., Clin. Cancer Res., 24, 4066-71 (2018); Konercy G. E. & Kristeleit R. S., Br. J. Cancer, 115, 1157-73 (2016); Zimmer A. S., et al., Curr. Treat. Options Oncol., 19, 21 (2018)) However, 40%-60% of cancers in patients with BRCA1 deficiency do not respond to PARP inhibitors (Audeh M. W., et al, Lancet., 276, 245-52 (2010); Tutt A., et al., Lancet., 376, 235-44 (2010); Fong P. C., et al., N Engl. J. Med., 361, 123-34 (2009)). Therefore, there remains a pressing need to identify additional targetable vulnerabilities in BRCA1-deficient cancers and to develop more effective combination therapeutic strategies to treat these cancers by, for example, inducing ferroptosis.

### SUMMARY

As described herein, a combination of a PARP inhibitor and a GPX4 inhibitor induce ferroptosis in cancers associated with BRCA1 deficiency. Further, a combination of GPX4 inhibitors with PARP inhibitors can be used to treat BRCA1 deficient cancers associated with BRCA1 deficiency.

The present disclosure provides a method for treating a BRCA1 deficient cancer, the method comprising administering to a subject a glutathione peroxidase 4 (GPX4) inhibitor and a poly (ADP-ribose) polymerase (PARP) inhibitor.

In some aspects, the subject has a BRCA1 mutation.

In some aspects, the subject does not have a BRCA2 deficiency.

In some aspects, the subject does not have a BRCA2 mutation.

In some aspects, the GPX4 inhibitor is selected from the group consisting of RSL3, ML162, ML210, JKE-1674, withaferin A, and a combination thereof.

In some aspects, the PARP inhibitor is selected from the group consisting of olaparib, niraparib, veliparib, talazoparib, rucaparib, fluzoparib, and a combination thereof.

In some aspects, the GPX4 inhibitor is JKE-1674, and the PARP inhibitor is olaparib. In some aspects, the GPX4 inhibitor is withaferin A, and the PARP inhibitor is olaparib. In some aspects, the GPX4 inhibitor is RSL3, and the PARP inhibitor is olaparib. In some aspects, the GPX4 inhibitor is RSL3, and the PARP inhibitor is niraparib.

In some aspects, the BRCA1 deficient cancer is a tumor.

In some aspects, the tumor is a carcinoma.

In some aspects, the BRCA1 deficient cancer is selected from the group consisting of breast cancer, ovarian cancer, colon cancer, pancreatic cancer, and prostate cancer.

In some aspects, the BRCA1 deficient cancer is breast cancer.

In some aspects, the BRCA1 deficient cancer has low GPX4 expression relative to non-deficient BRCA1 cancers.

In some aspects, the BRCA1 deficient cancer is not deficient of 53BP1.

In some aspects, the BRCA1 deficient cancer is deficient of 53BP1.

In some aspects, the GPX4 inhibitor is administered prior to administration of the PARP inhibitor.

In some aspects, the GPX4 inhibitor is administered after administration of the PARP inhibitor.

In some aspects, the GPX4 inhibitor and the PARP inhibitor are administered simultaneously.

In some aspects, the GPX4 inhibitor and the PARP inhibitor are administered in the same composition.

In some aspects, the GPX4 inhibitor and the PARP inhibitor are administered in different compositions.

In some aspects, the administration induces ferroptosis.

In some aspects, the administration induces cancer cell death and/or reduces cancer cell growth in the subject.

In some aspects, the subject is a human.

In some aspects, the present disclosure provides a method of treating a cancer in a subject in need thereof, comprising identifying whether the subject has a BRCA1 deficiency and administering to the subject having a BRCA1 deficiency a GPX4 inhibitor and a PARP inhibitor. The present disclosure also provides a method of selecting a subject afflicted with a cancer as suitable for treatment with a GPX4 inhibitor and a PARP inhibitor, the method comprising identifying the subject as having a BRCA1 deficiency and treating the subject with a GPX4 inhibitor and a PARP inhibitor.

In some aspects, identifying whether the subject has decreased expression of BRCA1 comprises obtaining a cancer sample from the subject and analyzing the sample for the BRCA1 expression level.

The present disclosure provides a method of selecting a subject afflicted with a cancer as suitable for treatment with a GPX4 inhibitor and a PARP inhibitor, the method comprising identifying the subject as having a BRCA1 mutation and treating the subject with a GPX4 inhibitor and a PARP inhibitor.

In some aspects, identifying whether the subject has a BRCA1 mutation comprises obtaining a cancer sample from the subject and analyzing the BRCA1 mutation status in the sample.

In some aspects, the subject does not have a BRCA2 deficiency.

In some aspects, the subject does not have a BRCA2 mutation.

In some aspects, the present disclosure provides a method of inducing ferroptosis in a BRCA1-deficient cancer cell, comprising contacting the cell with a GPX4 inhibitor and a PARP inhibitor.

In some aspects, the cancer cell is in a tumor.

In some aspects, the tumor is in a human.

In some aspects, the cancer is PARP inhibitor resistant. In some aspects, the cancer is PARP inhibitor sensitive.

In some aspects, the ferroptosis induction is increased compared to use of a GPX4 inhibitor alone or a PARP inhibitor alone.

In some aspects, cell death is increased compared to use of a GPX4 inhibitor alone or a PARP inhibitor alone.

In some aspects, cell viability is reduced compared to use of a GPX4 inhibitor alone or a PARP inhibitor alone.

In some aspects, lipid peroxidation in the cell is increased compared to use of a GPX4 inhibitor alone or a PARP inhibitor alone.

In some aspects, the GPX4 inhibitor is selected from the group consisting of RSL3, ML162, ML210, JKE-1674, withaferin A, and a combination thereof.

In some aspects, the PARP inhibitor is selected from the group consisting of olaparib, niraparib, veliparib, talazoparib, rucaparib, fluzoparib, and a combination thereof.

In some aspects, the GPX4 inhibitor is JKE-1674, and the PARP inhibitor is olaparib. In some aspects, the GPX4 inhibitor is withaferin A, and the PARP inhibitor is olaparib. In some aspects, the GPX4 inhibitor is RSL3, and the PARP inhibitor is olaparib. In some aspects, the GPX4 inhibitor is RSL3, and the PARP inhibitor is niraparib.

In some aspects, the methods described herein further comprise:
a) administering chemotherapy;
b) performing surgery;
c) administering radiation therapy;
d) administering targeted therapy; or
e) any combination thereof.

In some aspects of the methods described herein, the administering reduces the cancer burden.

The present disclosure also provides a pharmaceutical composition comprising a GPX4 inhibitor and a PARP inhibitor for use in treating a cancer cell.

In some aspects, the GPX4 inhibitor is selected from the group consisting of RSL3, ML162, ML210, JKE-1674, withaferin A, and a combination thereof.

In some aspects, the PARP inhibitor is selected from the group consisting of olaparib, niraparib, veliparib, talazoparib, rucaparib, fluzoparib, and a combination thereof.

In some aspects, the GPX4 inhibitor is JKE-1674, and the PARP inhibitor is olaparib. In some aspects, the GPX4 inhibitor is withaferin A, and the PARP inhibitor is olaparib. In some aspects, the GPX4 inhibitor is RSL3, and the PARP inhibitor is olaparib. In some aspects, the GPX4 inhibitor is RSL3, and the PARP inhibitor is niraparib.

In some aspects, the composition described herein further comprises at least one pharmaceutically acceptable excipient. In some aspects, at least one pharmaceutically acceptable excipient is a pharmaceutically acceptable carrier.

The present disclosure provides a method of of treating a PARP inhibitor-resistant cancer in a subject in need thereof, comprising administering an effective amount of a PARP inhibitor and GPX4 inhibitor to the subject, wherein the PARP inhibitor-resistant cancer is BRCA1 deficient.

In some aspects, the BRCA1 deficiency is a BRCA1 mutation.

In some aspects, the subject does not have a BRCA2 deficiency.

In some aspects, the subject does not have a BRCA2 mutation.

In some aspects, the GPX4 inhibitor is selected from the group consisting of RSL3, ML162, ML210, JKE-1674, withaferin A, and a combination thereof.

In some aspects, the PARP inhibitor is selected from the group consisting of olaparib, niraparib, veliparib, talazoparib, rucaparib, fluzoparib, and a combination thereof.

In some aspects, the GPX4 inhibitor is JKE-1674, and the PARP inhibitor is olaparib. In some aspects, the GPX4 inhibitor is withaferin A, and the PARP inhibitor is olaparib. In some aspects, the GPX4 inhibitor is RSL3, and the PARP inhibitor is olaparib. In some aspects, the GPX4 inhibitor is RSL3, and the PARP inhibitor is niraparib.

In some aspects, the PARP inhibitor-resistant cancer is a tumor.

In some aspects, the tumor is a carcinoma.

In some aspects, the PARP inhibitor-resistant cancer is selected from the group consisting of breast cancer, ovarian cancer, colon cancer, pancreatic cancer, and prostate cancer.

In some aspects, the PARP-resistant cancer is breast cancer.

### BRIEF DESCRIPTION OF THE DRAWINGS

FIG. 1A, FIG. 1B, FIG. 1C, FIG. 1D, FIG. 1E, FIG. 1F, FIG. 1G, FIG. 1H, FIG. 1I,

FIG. 1J, FIG. 1K, FIG. 1L, and FIG. 1M show BRCA1 deficiency promotes GPX4 inhibitor-induced ferroptosis. FIG. 1A is a graph that shows the percentage of cell death in different BRCA1 sgRNAs infected cells (HT1080, SKOV3, HEY, HS578T, and RPMI-7951) in the presence and absence of RSL3. FIG. 1B is a graph that shows the percentage of lipid peroxidation in BRCA1 sgRNA infected cells (HT1080, SKOV3, HEY, HS578T, and RPMI-7951) in the presence of RSL3. FIGS. 1C and 1D are plots that show the percentage of relative cell viability in BRCA1 sgRNAs infected HT1080, SKOV3, and DLD-1 cells in the presence of RSL3. FIG. 1E is a plot that shows the percentage of relative cell viability in BRCA1 reconstituted UWB1.289 cells in the presence of RSL3.

FIG. 1F is a graph that shows the percentage of cell death in the presence of RSL3, (RSL3+apoptosis inhibitor Z-VAD), (RSL3+ferroptosis inhibitor Fer-1), and (RSL3+iron chelator DFO) in BRCA1 sgRNAs infected SKOV3 cells. FIGS. 1G-1I are plots that show the percentage of relative cell viability in BRCA1 sgRNAs infected HT1080 cells treated with ML210, JKE-1674, or ML162, respectively. FIG. 1J is a graph showing the percentage of cell death in the presence of ML162 and (ML162+Fer-1) in BRCA1 sgRNAs infected SKOV3 cells. FIG. 1K is a graph showing the percentage of cell death in the presence of ML210 and (ML210+Fer-1) in BRCA1 sgRNAs infected SKOV3 cells. FIG. 1L is a graph showing the percentage of cell death in the presence of JKE-1674 and (JKE-1674+Fer-1) in BRCA1 sgRNAs infected SKOV3 cells. FIG. 1M is a plot that shows the percentage of relative cell viability in BRCA2 sgRNAs infected HT1080 cells in the presence of RSL3.

FIG. 2A, FIG. 2B, FIG. 2C, FIG. 2D, FIG. 2E, FIG. 2F, FIG. 2G, FIG. 2H, FIG. 2I, FIG. 2J, FIG. 2K, FIG. 2L, and FIG. 2M show BRCA1 deficiency promotes GPX4 inhibitor-induced ferroptosis via interference with GPX4 transcription. FIG. 2A provides images showing residual levels of GPX4 protein in cells (HT1080, HEY, SKOV3, HS578T, RPMI-7951, and DLD-1) infected with BRCA1 sgRNAs as determined by western blotting. FIG. 2B provides an image showing GPX4 protein levels in UWB1.289 cells reconsituted with BRCA1 expression as determined by western blotting. FIG. 2C provides an image showing GPX4 protein levels in HS578T cells infected with BRCA1 sgRNA or BRCA1 sgRNA infection supplemented with GPX4 as determined by western blotting. FIG. 2D is a plot showing the percentage of cell death in the presence of RSL3 in HS578T cells infected with BRCA1 sgRNA or BRCA1 sgRNA infection supplemented with GPX4. FIGS. 2E and 2F are plots showing GPX4 expression and GPX4 promoter activity in HS578T and 293T cells infected with BRCA1 sgRNAs. FIG. 2G shows plots of GPX4 expression in HT1080 and HS578T cells from control and BRCA1 sgRNA infection (Empty vector, BRCA1 WT, BRCA1 C61G, or BRCA1 M1775R). FIG. 2H provides images showing levels of BRCA1 and GPX4 protein in HT1080 and HS578T cells from BRCA1 sgRNA infection (Empty vector, BRCA1 wt, BRCA1 C61G, or BRCA1 M1775R). FIG. 2I is a plot showing GPX4 promoter activity in 293T cells infected with BRCA1 sgRNAs (Empty vector, BRCA1 wt, BRCA1 C61G, or BRCA1 M1775R). FIG. 2J is a plot showing the percentage of cell death in the presence of RSL3 in HS578T cells infected with BRCA1 sgRNA (Empty vector, BRCA1 wt, BRCA1 C61G, or BRCA1 M1775R). FIG. 2K is a graph of BRCA1 chromatin immunoprecipitation sequencing profiles in cells (SH-EP, HEPG2, and HeLa-S3) from GEO datasets (GSE31477 and GSE111905) showing the BRCA1 binding sites (BS) at the GPX4 promoter region. FIG. 2L is a plot showing chromatin immunoprecipitation analyses confirming the binding sites (BS) of BRCA1 at GPX4 promoter region in HS578T cells. FIG. 2M is an image showing the mechanism of how BRCA1 regulates GPX4 transcription and GPX4 inhibitor-induced ferroptosis.

FIG. 3A, FIG. 3B, FIG. 3C, FIG. 3D, FIG. 3E, FIG. 3F, FIG. 3G, FIG. 3H, FIG. 3I, FIG. 3J, FIG. 3K, and FIG. 3L show BRCA1 deficiency suppresses Erastin-induced ferroptosis via inhibiting without blocking sulfasalazine or cystine starvation. FIG. 3A are graphs showing the percentage of cell death in BRCA1 sgRNAs infected HT1080 and HEY cells in presence of Erastin. FIG. 3B provides graphs showing the relative cell viability of BRCA1 sgRNA infected HT1080 and HEY cells in the presence of increasing concentration of Erastin. FIG. 3C shows the percentage of lipid peroxidation in BRCA1 sgRNA infected HT1080 and HEY cells in the presence of Erastin. FIG. 3D is a graph showing the relative cell viability of BRCA1 sgRNA infected HT1080 cells in the presence of Erastin, (Erastin+Z-VAD), (Erastin+Fer-1), and (Erastin+DFO). FIG. 3E is a graph showing the relative cell viability of BRCA1 sgRNA infected HT1080 cells or BRCA1 sgRNA infected HT1080 cells with wild-type BRCA1 re-expression in the presence of Erastin. FIG. 3F provides plots showing the relative cell viability of BRCA1 sgRNA infected HT1080 and HEY cells in the presence of increasing concentration of imidazole ketone Erastin (IKE, an Erastin analog). FIG. 3G provides plots showing the relative cell viability of BRCA1 sgRNA infected HT1080 and HEY cells in the presence of increasing concentration of sulfasalazine. FIG. 3H are graphs showing the relative cell viability of BRCA1 sgRNAs infected HT1080 and HEY cells in the presence of cysteine.

FIGS. 31 and 3J provide plots showing the relative cell viability of BRCA2 sgRNA infected HT1080 cells in the presence of increasing concentration of Erastin or IKE. FIG. 3K provides a plot showing the relative cell viability of BRCA2 sgRNA infected HT1080 cells in the presence of increasing concentration of sulfasalazine. FIG. 3L is a graph showing the relative cell viability of BRCA2 sgRNAs infected HT1080 cells in the presence of cysteine.

FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 4M, FIG. 4N, and FIG. 4O show BRCA1 deficiency suppresses Erastin-induced ferroptosis via inhibiting VDAC3 expression and mitochondria-lipid peroxidation. FIG. 4A provides images showing the level of VDAC1, VDAC2, and VDAC3 in BRCA1 sgRNA infected cells as determined by western blotting. FIG. 4B is a graph showing the relative VDAC3 mRNA levels in BRCA1 sg RNA infected HT1080 cells as compared to normal HT1080 cells. FIG. 4C is an image showing no expression of VDAC3 in VDAC3 sgRNA transfected cells as examined by western blotting. FIGS. 4D and 4E are plots showing the relative cell viability of VDAC3 sgRNA transfected HT1080 cells in the presence of increasing concentration of Erastin or IKE. FIG. 4F is a graph showing lipid peroxidation in VDAC3 sgRNA infected cells in the presence of Erastin. FIG. 4G is a graph showing the relative cell viability of IDA (3 sgRNAs infected HT1080 cells in the presence of cysteine. FIG. 4H is a plot showing the relative cell viability of VDAC3 sgRNA transfected HT1080 cells in the presence of increasing concentration of sulfasalazine. FIG. 4I is an image showing expression of BRCA1 and VDAC3 in BRCA1 sgRNA infected cells, VDAC3 sgRNA infected cells, and BRCA1 sgRNA and VDAC3 sgRNA infected cells as determined by western blotting. FIG. 4J is a plot showing the relative cell viability of VDAC3 sgRNA infected cells, BRCA1 sgRNA infected cells, and VDAC3 sgRNA and BRCA1 sgRNA infected cells in the presence of increasing concentration of Erastin. FIG. 4K is a graph of BRCA1 chromatin immunoprecipitation sequencing profiles in cells (GM12878, H1-hESC, HeLa-S3, and HEPG2) from GEO datasets (GSE31477) showing a sharp peak of BRCA1 binding at the site of the IDA (3 gene promoter. FIG. 4L is a plot showing chromatin immunoprecipitation analyses confirming the BRCA1 binding on VDAC3 promoter in HT1080 cells. FIGS. 4M and 4N are graphs showing mitochondrial lipid peroxidation of BRCA1 sgRNA or VDAC3 sgRNA infected cells in the presence of Erastin. FIG. 4O is a graph showing the relative cell viability of BRCA1 sgRNA or VDAC3 sgRNA infected cells in the presence of Erastin, (Erastin+mitochondrial ROS scavenger mitoTEMPO), (Erastin+ROS scavenger TEMPO), or (Erastin+Fer-1).

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5L, FIG. 5M, FIG. 5N, FIG. 5O, FIG. 5P, FIG. 5Q, FIG. 5R, FIG. 5S, and FIG. 5T show that PARP inhibitors (PARPi) synergize with GPX4 inhibitors (GPX4i) in BRCA1-deficient cancers via ferroptosis. FIGS. 5A and 5B provide plots showing the relative cell viability of UWB1.289 cells (control and BRCA1-reconsituted) treated with PARPi (olaparib or niraparib) and/or RSL3. FIGS. 5C and 5D provide plots of relative cell viability of HS578T cells (BRCA1 sgRNA infected and control) treated with olaparib and/or RSL3. FIG. 5E is a graph of scores from the Bliss independence model indicating the combination effects of PARPi and RSL3 in cells featured in FIGS. 5A-D. FIGS. 5F and 5G provide plots showing the relative cell viability of cells (SUM149, HCC1937, HCC1395, SUM1315, MCF10A, MDA-MB-468, HCC1806, and MDA-MB-453) treated with olaparib and/or RSL3. FIG. 5H is a graph of scores from the Bliss independence model indicating the combination effects of olaparib and RSL3 in cells featured in FIGS. 5F and 5G. FIG. 5I provides graphs of percent cell death in cells (UWB1.289, HCC1937, and SUM149) cells treated with olaparib and/or RSL3 in the absence or presence of ferrostatin-1. FIG. 5J provides a graph of percent cell death in HCC1937 cells treated with olaparib and/or RSL3 in the absence or presence of Z-VAD, Nec-1s, or liproxstatin-1.

FIG. 5K provides a graph of percent cell death in SKOV3 cells (control and BRCA1 sgRNA infected) treated with olaparib, niraparib, and/or JKE-1674 in the absence or presence of ferrostatin-1. FIG. 5L provides graphs of percent cell death in cells (MCF10A, HS578T, SUM149, and HCC1937) olaparib and/or JKE-1674 in the absence or presence of ferrostatin-1. FIGS. 5M and 5N are plots showing the tumor volume of SKOV3 xenografts (control or BRCA1 sgRNA infected) over 24 days and treated with olaparib and/or JKE-1674 in the absence or presence of liproxstatin-1. FIG. 5O provides images that show BRCA1 and GPX4 protein levels in SUM149 cells (parental or BRCA1-reverted clones) as determined by western blotting. FIG. 5P is a plot showing relative cell viability in SUM149 cells (parental or BRCA1-reverted clones) treated with RSL3. FIGS. 5Q-5T provide plots showing relative cell viability in SUM149 cells (parental or BRCA1-reverted clones) treated with PARPi (olaparib or niraparib) and/or RSL3.

FIG. 6A, FIG. 6B, FIG. 6C, FIG. 6D, FIG. 6E, FIG. 6F, FIG. 6G, FIG. 6H, FIG. 6I, FIG. 6J, FIG. 6K, FIG. 6L, FIG. 6M, FIG. 6N, FIG. 6O, FIG. 6P, FIG. 6Q, FIG. 6R, FIG. 6S, FIG. 6T, FIG. 6U, FIG. 6V, and FIG. 6W show NCOA4-mediated ferritinophagy coupled with defective GPX4 induction contributes to the synergy of PARP inhibitors and GPX4 inhibitors in BRCA1-deficient cancer cells. FIGS. 6A-6D are images showing protein levels of NCOA4, L3CI and L3CII in cells (HCC1937 and SUM149) when treated with olaparib (FIGS. 6A and 6C) or niraparib (FIGS. 6B and 6D) as determined by western blotting. FIG. 6E is graph showing the relative NCOA4 levels in HCC1937 treated with olaparib or niraparib. FIG. 6F is graph showing the relative NCOA4 levels in SUM149 cells treated with olaparib or niraparib. FIG. 6G is graph showing the relative labile iron pool in HCC1937 cells treated with olaparib or niraparib. FIG. 6H is graph showing the relative labile iron pool in SUM149 cells treated with olaparib or niraparib. FIG. 6I provides an image showing the protein levels of NCOA4 in HCC1937 cells (control and NC (A4 sgRNA infected) treated with olaparib as determined by western blotting. FIG. 6J provides a graph showing the relative labile iron pool in HCC1937 cells (control and NCOA4 sgRNA infected) treated with olaparib. FIG. 6K provides a graph showing the percent of lipid peroxidation in HCC1937 cells (control and NCO4 sgRNA infected) treated with olaparib. FIG. 6L is a graph showing percent cell death in HCC1937 cells (control and NC (A4 sgRNA infected) treated with olaparib and/or RSL3. FIG. 6M is a graph showing percent of lipid peroxidation in HCC1937 cells treated with olaparib or niraparib in the absence or presence of deferoxamine (DFO). FIG. 6N provides a graph showing percent cell death in HCC1937 cells treated with olaparib, niraparib, and/or RSL3 in the absence or presence of DFO. FIG. 6O provides images showing protein levels of NCOA4, L3CI and L3CII in HS578T cells (BRCA1 sgRNA infected and control) treated with olaparib. FIG. 6P are graphs showing the relative labile iron pool in HS578T cells (BRCA1 sgRNA infected and control) treated with olaparib. FIG. 6Q provides an image showing protein levels of GPX4 and p-H2AX in HS578T cells (BRCA1 sgRNA infected and control) treated with olaparib. FIG. 6R provides an image showing protein levels of GPX4 and p-H2AX in cells (HCC1937, SUM149, and HS578T) treated with olaparib. FIG. 6S provides an image showing protein levels of GPX4 in HS578T cells (control and GPX4 sgRNA infected). FIG. 6T is a graph showing percent cell death in HS578T cells (control and GPX4 sgRNA infected) treated with olaparib in the absence or presence of ferrostatin-1. FIG. 6U provides an image showing protein levels of GPX4 in SUM149 cells (EV and GPX4-expressing). FIG. 6V is a graph showing the percent cell death of SUM149 cells (EV and GPX4-expressing) treated with olaparib in the absence or presence of RSL3. FIG. 6W are schemes illustrating different levels of ferroptosis induced by PARPi alone in BRCA1 WT and in BRCA1 deficient cells; by PARPi and GPX4i in BRCA1 WT and BRCA1 deficient cells.

FIG. 7A, FIG. 7B, FIG. 7C, FIG. 7D, FIG. 7E, FIG. 7F, FIG. 7G, FIG. 7H, FIG. 7I, FIG. 7J, FIG. 7K, FIG. 7L, FIG. 7M, FIG. 7N, and FIG. 7O show GPX4 inhibitors (GPX4i) overcome resistance to PARP inhibitors (PARPi) in BRCA1-mutant tumors. FIG. 7A provides a schematic of the orthotopic implantation model for cell line-derived xenografts (CDXs) and patient-derived xenografts (PDXs). FIGS. 7B-7D provides plots showing tumor volume growth of HCC1937-derived xenografts (7B), PDX 18-S (7C), and PDX 27-S (7D) after treatment with olaparib, JKE-1674, a combination of olaparib and JKE-1674, a combination of olaparib, JKE-1674, and liproxstatin (Lip-1). FIGS. 7E and 7F provide plots showing percent survival and tumor volume growth in PDX 18-S after treatment with talazoparib and/or JKE-1674. FIGS. 7G and 7H provide plots showing percent survival and tumor volume growth in PDX 27-S after treatment with talazoparib and/or JKE-1674. FIG. 7I is a plot showing tumor volume growth in PDX PIM224 (BRCA1 WT) treated with olaparib, JKE-1674, a combination of olaparib and JKE-1674, a combination of olaparib, JKE-1674, and liproxstatin (Lip-1). FIGS. 7J and 7K provides GPX4 immunochemistry staining images (7J) and a GPX4 staining score graph (7K) of PDX 18-S, PDX 27-S, and PDX PIM224. FIGS. 7L and 7M provide graphs showing immunochemistry staining scoring of 4-HNE (7L) and p-H2AX (7M) from PDX 18-S, PDX 27-S, and PDX PIM224 treated with olaparib, JKE-1674, a combination of olaparib and JKE-1674, a combination of olaparib, JKE-1674, and liproxstatin (Lip-1). FIGS. 7N and 7O provide schematics showing the dual role of BRCA1 in ferroptosis regulation by governing GPX4 or IDA (3 transcription (7N) and showing the vulnerability of BRCA1-deficient cancer to PARP and GPX4 co-inhibition and its underlying mechanisms (7O).

FIG. 8A, FIG. 8B, FIG. 8C, FIG. 8D, FIG. 8E, FIG. 8F, FIG. 8G, FIG. 8H, FIG. 8I, FIG. 8J, FIG. 8K, FIG. 8L, FIG. 8M, FIG. 8N, FIG. 8O, FIG. 8P, FIG. 8Q, FIG. 8R, FIG. 8S, and FIG. 8T show BRCA1 deficiency promotes GPX4 inhibitor-induced ferroptosis. FIG. 8A provides a schematic depicting ferroptosis pathways and ferroptosis inducers (FINs) used throughout the Figures. FIGS. 8B and 8C provides images showing residual levels of BRCA1 protein in cells (HT1080, SKOV3, HEY, HS578T, and RPMI-7951 and DLD-1) infected with BRCA1 sgRNAs as determined by western blotting. FIG. 8D are plots showing the relative cell viability in SKOV3 and HT1080 cells (control and BRCA1 sgRNA infected) treated with olaparib. FIG. 8E are plots showing the relative cell viability in SKOV3 and HT1080 cells (control and BRCA1 sgRNA infected) treated with ferrostatin-1 and RSL3. FIGS. 8F-8K provide plots showing the relative cell proliferation of control and BRCA1 sgRNA infected cells (HT1080, SKOV3, HEY, HS578T, RPMI-7951, and DLD1). FIG. 8L is a plot showing the relative cell proliferation of UWB1.289 cells (control and BRCA1-reconsituted). FIG. 8M is an image showing the protein levels of ECAD in HT1080 cells (control and ECAD) sgRNA infected) as determined by western blotting. FIG. 8N provides a plot showing relative cell viability in HT1080 cells (control and ECAD sgRNA infected) treated with RSL3. FIG. 8O is an image showing the protein levels of BRCA2 in HT1080 cells (control and BRCA2 sgRNA infected) as determined by western blotting. FIGS. 8P-8R provide plots showing relative cell viability in HT1080 cells (control and BRCA2 sgRNA infected) treated with ML210 (8P), JKE1674 (8Q), and ML162 (8R). FIGS. 8S and 8T are plots showing the Pearson correlation z-scores for BRCA1 and BRCA2 from Therapeutics Response Portal database.

FIG. 9A, FIG. 9B, FIG. 9C, and FIG. 9D show BRCA1 regulates GPX4 through the BRCT domain. FIG. 9A provides an image showing the protein levels of ACSL4, FSP1, and DHODH in HT1080 cells (control and BRCA1 sgRNA infected) as determined by western blotting. FIG. 9B provides an image showing the protein levels of GPX4 in HT1080 cells (control and BRCA2 sgRNA infected) as determined by western blotting.

FIG. 9C provides a graph showing GPX4 expression in RPMI-7951 BRCA1 sgRNA infected cells expressing empty vector, WT BRCA1 construct, or mutant BRCA1 construct (C61G or M1775R). FIG. 9D provides an image showing the protein levels of BRCA1 and GPX4 in RPMI-7951 BRCA1 sgRNA infected cells expressing empty vector, WT BRCA1 construct, or mutant BRCA1 construct (C61G or M1775R) as determined by western blotting.

FIG. 10A, FIG. 10B, FIG. 10C, FIG. 10D, FIG. 10E, FIG. 10F, FIG. 10G, FIG. 10H, FIG. 10I, FIG. 10J, FIG. 10K, FIG. 10L, FIG. 10M, FIG. 10N, FIG. 10O, FIG. 10P, FIG. 10Q, and FIG. 10R show BRCA1 deficiency suppresses Erastin-induced ferroptosis via interference with VDAC3 transcription and mitochondrial lipid peroxidation. FIG. 10A provides an image showing the protein levels of SLC7A11 in HT1080 cells (control and BRCA1 sgRNA infected) as determined by western blotting. FIG. 10B is a graph showing relative GSH levels in HT1080 cells (control and BRCA1 sgRNA infected) treated with Erastin. FIGS. 10C and 10D provide images showing the protein levels of VDAC3, VDAC2, and, VDAC1 in HEY cells that are BRCA1 sgRNA infected (10C) and HT1080 cells that are BRCA2 sgRNA infected (10D) as determined by western blotting. FIG. 10E provides an image showing the protein levels of VDAC3 in HEY cells (control and VDA (3 sgRNA infected) as determined by western blotting. FIG. 10F is a graph showing percent cell death in HEY cells (control and VDA (3 sgRNA infected) treated with Erastin or IKE. FIG. 10G is a graph showing the relative cell viability in HEY cells (control and VDA (3 sgRNA infected) cultured in cystine-free medium. FIG. 10H is a plot showing the relative cell viability in HEY cells (control and VDAC3 sgRNA infected) treated with sulfasalazine. FIG. 10I provides an image showing the protein levels of VDAC2 and VDAC3 in HT1080 VDA (3 sgRNA infected cells expressing empty vector, VDAC2, and VDAC3 as determined by western blotting. FIG. 10J provide a plot showing relative cell viability in HT1080 VDAC3 sgRNA infected cells (expressing empty vector, VDAC2, and VDAC3) treated with Erastin. FIG. 10K is a graph showing VDAC3 expression in HT1080 BRCA1 sgRNA infected cells expressing empty vector, WT BRCA1 construct, or mutant BRCA1 construct (C61G or M1775R). FIG. 10L provides an image showing the protein levels of BRCA1 and VDAC3 in HT1080 BRCA1 sgRNA infected cells expressing empty vector, WT BRCA1 construct, or mutant BRCA1 construct (C61G or M1775R) as determined by western blotting. FIG. 10M provides a graph showing relative cell viability in HT1080 BRCA1 sgRNA infected cells expressing empty vector, WT BRCA1 construct, or mutant BRCA1 construct (C61G or M1775R) and treated with Erastin. FIGS. 10N and 10O are graphs showing the mitochondrial lipid peroxidation in control, BRCA1 sgRNA infected (10N), or VDAC3 sgRNA infected (10O) HEY cells treated with Erastin. FIG. 10P is a graph showing the mitochondrial lipid peroxidation in control, BRCA1 sgRNA infected, or VDAC3 sgRNA infected HT1080 cells treated with RSL3. FIG. 10Q provides a graph showing the percent cell death in HEY cells (control and IT) A (′3 sgRNA infected) treated with RSL3. FIG. 10R provides a plot showing the relative cell viability in HT1080 cells (control and VDAC3 sgRNA infected) treated with RSL3.

FIG. 11A, FIG. 11B, FIG. 11C, FIG. 11D, FIG. 11E, FIG. 11F, FIG. 11G, FIG. 11H, and FIG. 11I show PARP inhibitors synergize with GPX4 inhibitors in BRCA1-deficient cancers via ferroptosis. FIG. 11A provides an image showing the protein levels of GPX4 in BRCA1-WT (MCF10A, HS578T, MDA-MB-468, HCC1806, and MDA-MB-453) and BRCA1-deficient (SUM149, SUM1315, HCC1395, and HCC1937) cell lines as determined by western blotting. FIG. 11B provides graphs showing percent cell death in UWB1.289, HCC1937, and SUM149 cells treated with niraparib and/or RSL3 and in the absence or presence of ferrostatin-1. FIG. 11C provides graphs showing percent lipid peroxidation in HCC1937 cells treated with PARPi (olaparib or niraparib) and/or RSL3 and in the absence or presence of ferrostatin-1. FIG. 11D provides graphs showing percent lipid peroxidation in HS578T and SKOV3 cells (control and BRCA1 sgRNA infected) treated with niraparib and/or RSL3 and in the absence or presence of ferrostatin-1. FIG. 11E provides graphs showing percent cell death in HS578T and SKOV3 cells (control and BRCA1 sgRNA infected) treated with niraparib and/or RSL3 and in the absence or presence of ferrostatin-1. FIGS. 11F-11H provide plots showing relative cell viability of UWB1.289 (11F), HCC1937 (11G), and HS578T (11H) cells treated with RSL3 and olaparib. FIG. 11I is a plot showing the bliss scores of olaparib and RSL3 treated UWB1.289, HCC1937, and HS578T cells.

FIG. 12A, FIG. 12B, FIG. 12C, FIG. 12D, FIG. 12E, FIG. 12F, FIG. 12G, FIG. 12H, FIG. 12I, FIG. 12J, FIG. 12K, FIG. 12L, and FIG. 12M show PARP inhibitors combined with GPX4 inhibitors in BRCA1-mutant/deficient or homologous-recombination-restored cells or tumors. FIG. 12A is a plot showing the body weight of mice treated with olaparib and/or JKE-1674 and in the absence or presence of ferrostatin-1. FIGS. 12B and 12C provide p-H2AX (12B) and RAD51 (12C) immunochemistry staining images of SKOV3 cells (control and BRCA1 sgRNA infected) treated with olaparib and/or JKE-1674 and in the absence or presence of ferrostatin-1. FIGS. 12D and 12E are plots showing p-H2AX (12D) and RAD51 (12E) immunochemistry staining scores of SKOV3 cells (control and BRCA1 sgRNA infected) treated with olaparib and/or JKE-1674 and in the absence or presence of ferrostatin-1. FIG. 12F provides 4-HNE immunochemistry staining images of SKOV3 cells (control and BRCA1 sgRNA infected) treated with olaparib and/or JKE-1674 and in the absence or presence of ferrostatin-1. FIG. 12G provides 4-hydroxynonenal (4-HNE) immunochemistry staining score of SKOV3 cells (control and BRCA1 sgRNA infected) treated with olaparib and/or JKE-1674 and in the absence or presence of ferrostatin-1. FIG. 12H is a plot showing the relative cell viability of BRCA1-reverted SUM 149 cells (clones 2, 5, and 8) treated with olaparib. FIG. 12I is a plot showing the relative cell proliferation of BRCA1-reverted SUM 149 cells (clones 2, 5, and 8). FIG. 12J provides graphs showing the bliss scores of olaparib and RSL3 treated BRCA-reverted SUM 149 cells (clones 2, 5, and 8). FIG. 12K provides an image showing the protein levels of 53BP1 and GPX4 in UWB1.289 cells (control and 53BP1 sgRNA infected) as determined by western blotting. FIG. 12L provides plots showing the relative cell viability of UWB1.289 cells (control and 53BP1 sgRNA infected) treated with RSL3 and/or with olaparib. FIG. 12M is a graph showing the bliss scores of olaparib and RSL3 treatment in UWB1.289 cells (control and 53BP1 sgRNA infected).

FIG. 13A, FIG. 13B, FIG. 13C, FIG. 13D, FIG. 13E, FIG. 13F, FIG. 13G, FIG. 13H, FIG. 13I, FIG. 13J, and FIG. 13K show NCOA4-mediated ferritinophagy coupled with defective GPX4 induction contributes to the synergy of PARP inhibitors and GPX4 inhibitors in BRCA1-deficient cancer cells. FIG. 13A provides an image showing the protein levels of ACSL4, SLC7A11, GPX4, FSP1, and DHODH in HCC1937 cells treated with olaparib as determined by western blotting. FIG. 13B provides a graph showing the percent lipid peroxidation of HCC1937 cells (control and NCO) A4 sgRNA infected) treated with niraparib. FIG. 13C provides a graph showing percent cell death in HCC1937 cells (control and NC (A4 sgRNA infected) treated with RSL3 and/or niraparib. FIG. 13D provides a graph showing percent lipid peroxidation in SUM149 cells treated with a PARPi (olaparib or niraparib) in the absence or presence of Deferoxamine (DFO). FIG. 13E provides a graph showing percent cell death in SUM149 cells treated with a PARPi (olaparib or niraparib) and/or RSL3 in the absence or presence of DFO. FIGS. 13F-13H provides graphs showing the cell viability in SUM149 cells and HS578T cells (control and BRCA1 sgRNA infected) treated with olaparib in the absence or presence of 5 μM ferrostatin-1. FIG. 13I provides graphs showing the percent reactive oxygen species (ROS) in HS578T (control, BRCA1 sgRNA infected, and WT), SUM149, and HCC1937 cells treated with olaparib. FIG. 13J provides an image showing the protein levels of GPX4 in HS578T cells treated with olaparib and/or N-acetyl-1-cysteine (NAC) as determined by western blotting. FIG. 13K provides a graph showing cell viability of SUM149 cells treated with olaparib and/or N-acetyl-1-cysteine (NAC).

FIG. 14A, FIG. 14B, FIG. 14C, FIG. 14D, FIG. 14E, FIG. 14F, FIG. 14G, FIG. 14H, FIG. 14I, FIG. 14J, FIG. 14K, FIG. 14L, and FIG. 14M show GPX4 inhibitors overcome resistance to PARP inhibitors in BRCA1-mutant tumors. FIGS. 14A-14C provide plots of tumor weights from tumors derived from HCC1937 cells, PDX 18-S, and PDX 16 treated with olaparib and/or JKE-1674 in the absence or presence of Lip-1. FIG. 14D provides a plot for the tumor volume growth from PDX 16 grown in mice and treated with olaparib and/or talazoparib. FIG. 14E provides a plot for the tumor weight from PDX PIM224 (BRCA1 WT) grown in mice and treated with olaparib and/or JKE-1674 in the absence or presence of Lip-1. FIGS. 14F-14H provide 4-HNE (14F), p-H2AX (14G) and cleaved caspase-3 (14H) immunochemistry staining images from PDX 18-S, PDX 27-S, and PDX PIM224 treated with olaparib and/or JKE-1674 and in the absence or presence of Lip-1. FIG. 14I provides plots showing cleaved caspase-3 staining scores from PDX 18-S, PDX 27-S, and PDX PIM224 treated with olaparib and/or JKE-1674 and in the absence or presence of Lip-1. FIGS. 14J-14M provide the body weight of mice growing xenografts from HCC1937 (14J) or PDXs from PDX 18-S (14K), PDX 27-S (14L), and PDX PIM224 (14M) all of which were treated with olaparib and/or JKE-1674 and in the absence or presence of Lip-1.

FIG. 15A, FIG. 15B, FIG. 15C, FIG. 15D, FIG. 15E, FIG. 15F, FIG. 15G, FIG. 15H, FIG. 15I, FIG. 15J, FIG. 15K, FIG. 15L, FIG. 15M, FIG. 15N, FIG. 15O, FIG. 15P, FIG. 15Q, FIG. 15R, FIG. 15S, FIG. 15T, FIG. 15U, and FIG. 15V show BRCA1 deficiency impairs NRF2-mediated GPX4 transcription by interacting with NRF2 via BRCA1's BRCT domain. FIG. 15A is a scheme showing the mutated regions of the different GPX4 promoter truncating mutants used in the experiments. FIG. 5B is a graph showing the fold change of GPX4 promoter activity in 293T cells carrying different GPX4 promoter truncating mutants. FIG. 15C is a scheme showing the mutations of the antioxidant response element (ARE) at the −1,800 to −800 nt region in different GPX4 promoter truncating mutants. FIG. 15D is a graph showing the fold change of GPX4 promoter activity in 293T cells carrying different GPX4 promoter truncating mutants. FIG. 15E provides images showing the expression levels of NRF2 and GPX4 in NRF2 sgRNA infected HS578T, SKOV3, and RPMI-7951 cells as determined by western blotting. FIG. 15F is a graph showing the fold change of NRF2 expression in BRCA1 sgRNA infected HS578T cell variants expressing empty vector, BRCA1 wild type, BRCA1 C61G, or BRCA1 M1775R as determined by qPCR. FIG. 15G is an image showing expression level of BRCA1 and NRF2 in BRCA1 sgRNA infected HS578T cells expressing empty vector, BRCA1 wild type, BRCA1 C61G, or BRCA1 M1775R as determined by western blotting. FIG. 15H is a graph showing the fold change of NRF2 expression in BRCA1 sgRNA infected HT1080 cell variants expressing empty vector, BRCA1 wild type, BRCA1 C61G, or BRCA1 M1775R as determined by qPCR. FIG. 15I is an image showing the expression of BRCA1, NRF2, and GPX4 in BRCA1 sgRNA infected HT1080 cell variants expressing empty vector, BRCA1 wild type, BRCA1 C61G, or BRCA1 M1775R as determined by western blotting. FIG. 15J is a graph showing the fold change of NRF2 expression in BRCA1 sgRNA infected RPMI-7951 cell variants expressing empty vector, BRCA1 wild type, BRCA1 C61G, or BRCA1 M1775R as determined by qPCR. FIG. 15K is an image showing the expression of BRCA1, NRF2, and GPX4 in BRCA1 sgRNA infected RPMI-7951 cell variants expressing empty vector, BRCA1 wild type, BRCA1 C61G, or BRCA1 M1775R as determined by western blotting. FIG. 15L is an image showing interaction of BRCA1 with NRF2 in BRCA1 sgRNA infected HS578T cell variants expressing empty vector, BRCA1 wild type, BRCA1 C61G, or BRCA1 M1775R as determined by immunoprecipitation. FIG. 15M is a graph showing the relative cell viability in BRCA1 sgRNA infected HT1080 cell variants expressing empty vector, BRCA1 wild type, BRCA1 C61G, or BRCA1 M1775R in the presence of Erastin. FIG. 15N is a graph showing the fold change of VDAC3 expression in BRCA1 sgRNA infected HT1080 cell variants expressing empty vector, BRCA1 wild type, BRCA1 C61G, or BRCAIM1775R as determined by qPCR. FIG. 15O is an image showing the expression of BRCA1 and VDAC3 in BRCA1 sgRNA infected HT1080 cell variants expressing empty vector, BRCA1 wild type, BRCA1 C61G, or BRCA1 M1775R as determined by western blotting. FIGS. 15P and 15Q are graphs showing the fold change of NRF2 promoter enrichment (15P) and GPX4 promoter enrichment (15Q) in BRCA1 sgRNA infected HS578T cells expressing empty vector, BRCA1 wild type, BRCA1 C61G, or BRCA1 M1775R in the presence of anti-BRCA1 or anti-NRF2. FIG. 15R is an image showing expression of NRF2, GPX4, and VDAC3 in BRCA2 sgRNA infected HT1080 cells as determined by western blotting. FIG. 15S is an image showing the expression of NRF2 and GPX4 in BRCA1 mutant SUM149 cells induced by tert-butylhydroquinone (TBHQ) or sulforaphane (SFN) as determined by western blotting. FIG. 15T is an image showing expression of NRF2 and GPX4 in NRF2 sgRNA infected SKOV3 cells, BRCA1 sgRNA infected SKOV3 cells, or BRCA1 sgRNA infected cells induced by TBHQ. FIG. 15U is a graph showing the percentage of cell death in NRF2 sgRNA infected SKOV3 cells, BRCA1 sgRNA infected SKOV3 cells, or BRCA1 sgRNA infected cells induced by TBHQ. FIG. 15V is a scheme illustrating BRCA1 modulation of GPX4 expression through interacting with NRF2.

FIG. 16A, FIG. 16B, FIG. 16C, FIG. 16D, FIG. 16E, FIG. 16F, FIG. 16G, FIG. 16H, FIG. 16I, FIG. 16J, FIG. 16K, FIG. 16L, FIG. 16M, and FIG. 16N show BRCA1 regulates GPX4 transcription through BRCA1 interactions with NRF2. FIG. 16A is graph showing the percentage of lipid peroxidation in SUM149 cells treated with niraparib or a combination of niraparib and DFO. FIGS. 16B and 16C are images showing expression of GPX4 and NRF2 in control HS578T (BRCA1-WT) cells, BRCA1 sgRNA infected HS578T cells, BRCA1-mutant HCC1937 cells, or BRCA1-mutant SUM149 cells as determined by western blotting. FIG. 16D is an image showing decreased expression of GPX4 in NRF2 sgRNA infected HS578T cells as compared to normal HS578T cells. FIG. 16E is graph showing the percentage of cell death in SUM149 cells treated with or without TBHQ in the presence of olaparib, RSL3, or a combination of olaparib and RSL3. FIG. 16F is graph showing the percentage of cell death in control or NRF2 sgRNA infected HS578T cells treated with olaparib, RSL3, or a combination of olaparib and RSL3. FIG. 16G is a graph showing the percentage of ROS in BRCA1 sgRNA infected HS578T cells treated with niraparib. FIG. 16H is an image showing expression of NRF2 and GPX4 in in HS578T cells treated with N-acetyl-1-cysteine (NAC), olaparib, or a combination of NAC and olaparib as determined by western blotting. FIGS. 16I and 16J are plots showing cell viability of HS578T cells, SUM149 cells, or BRCA1 sgRNA infected HS578T cells treated with ROS inducer TBH. FIGS. 16K to 16N are schemes illustrating different levels of ferroptosis induced by PARPi alone in BRCA1 WT (FIG. 16K), in BRCA1 mutant (FIG. 16L), by PARPi and GPX4i in BRCA1 WT (FIG. 16M), and by both PARPI and GPX4i in BRCA1 mutant (FIG. 16N).

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a,” “an,” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

The term “about,” as used herein, refers to a value or composition that is within an acceptable error range for the particular value or composition as determined by one of ordinary skill in the art, which will depend in part on how the value or composition is measured or determined, i.e., the limitations of the measurement system. For example, about can include the recited number ±10% (for example, “about 10” means 9 to 11).

The term “administering,” as used herein, refers to the physical introduction of a composition comprising a therapeutic agent (e.g., a GPX4 inhibitor and a PARP inhibitor) to a subject, using any of the various methods and delivery systems known to those skilled in the art. Routes of administration include oral, intravenous, intramuscular, subcutaneous, intraperitoneal, spinal or other parenteral routes of administration, for example by injection or infusion.

The term an “anti-cancer agent” or combination thereof promotes cancer regression in a subject. In some aspects, a therapeutically effective amount of the therapeutic agent promotes cancer regression to the point of eliminating the cancer.

The terms “sample,” “biological sample,” or a “cancer sample” as used herein refers to biological material isolated from a subject. The sample can contain any biological material suitable for determining gene expression, for example, by sequencing nucleic acids, protein expression, or any other marker of interest.

The sample can be any suitable biological tissue, for example, cancer tissue. In one aspect, the sample is a tumor tissue biopsy, e.g., a formalin-fixed, paraffin-embedded (FFPE) tumor tissue or a fresh-frozen tumor tissue or the like. In another aspect, an intratumoral sample is used. In another aspect, biological fluids can be present in a tumor tissue biopsy, but the biological sample will not be a biological fluid per se.

The term a “cancer,” as used herein, refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. The term “tumor” refers to a solid cancer. The term “carcinoma” refers to a cancer of epithelial origin.

The term “control sample,” as used herein refers to a biological sample (e.g. blood, urine, tumor) obtained from a “normal” or “healthy” individual(s) that is believed not to have cancer or from a “normal” or “healthy” (e.g., non-cancerous) biological sample from an individual(s) that is believed to have cancer. Controls may be selected using methods that are well known in the art. Once a level has become well established for a control population, array results from test biological samples can be directly compared with the known levels.

The term “BRCA1,” as used herein, refers to breast cancer type 1 susceptibility protein that is encoded by the BRCA1 gene (NCBI gene access code: NG_005905). BRCA1 protein (NCBI protein access code: AAC37594.1) contains three important domains that mediate its canonical functions in DNA damage response and repair (Huen M. S., et al., Nat. Rev. Mol. Cell Biol., 11, 138-48 (2010); Jiang, Q. & Greenberg, R. A., J. Biol. Chem., 290, 17724-32 (2015)): the N-terminal RING domain has the E3 ubiquitin ligase activity and mediates BRCA1 interaction with BARD1; the C-terminal BRCT domain associates with three distinctive complexes (BRCA1-A, -B, -C) and regulates different aspects of DNA damage response; the coiled-coil domain mediates BRCA1 interaction with the PALB2-BRCA2 complex, which then recruits the recombinase RAD51 for homologous recombination-mediated DNA repair. BRCA1 also regulates other cellular processes, such as gene transcription (Savage K. I. & Harkin D. P., FEBS J., 282, 630-46 (2015)).

The term “BRCA2,” as used herein, refers to breast cancer type 2 susceptibility protein that is encoded by the BRCA2 gene (NCBI gene accession code: NG_012772; NCBI protein accession code: KAI4062990).

The term “BRCA1 deficient,” as used herein, refers to reduced or eliminated BRCA1 polypeptide expression and/or reduced or eliminated BRCA1 polypeptide activity. A reduced level of BRCA1 polypeptide expression or BRCA1 polypeptide activity refers to any level of BRCA1 polypeptide expression or BRCA1 polypeptide activity that is lower than the median level of BRCA1 polypeptide expression or BRCA1 polypeptide activity typically observed in a sample (e.g., a control sample) from one or more healthy subjects (e.g., healthy humans) and/or from one or more healthy tissues (e.g., healthy human tissues). Control samples can include, without limitation, samples from subjects that do not have cancer, cell lines originating from subjects that do not have cancer, non-tumorigenic cell lines, and adjacent normal tissue. It will be appreciated that comparable samples are used when determining whether or not a particular level is a reduced level. An eliminated level of BRCA1 polypeptide expression or BRCA1 polypeptide activity refers to any non-detectable level of BRCA1 polypeptide expression or BRCA1 polypeptide activity.

The term “BRCA1 mutation” refers to one or more modifications in a BRCA1 nucleic acid (e.g., a nucleic acid encoding a BRCA1 polypeptide) and/or one or more modifications in a BRCA1 polypeptide that alter cancer cell metabolism. A modification in a BRCA1 polypeptide can alter any appropriate type of cancer cell metabolism. In some aspects, a modification does not affect glycolysis. Examples of metabolic alterations that can be seen in cells in a BRCA1-deficient cancer include, without limitation, reduced levels of adenosine-5′-triphosphate (ATP), increased levels of reactive oxygen species (ROS), and reduced OXPHOS. In some cases, a BRCA1-deficient cancer can include one or more cancer cells having one or more modifications in a BRCA1 nucleic acid and/or one or more modifications in a BRCA1 polypeptide that can reduce OXPHOS. A modification can be any appropriate modification. A modification in a BRCA1 nucleic acid or a modification in a BRCA1 polypeptide refers to any change in a BRCA1 nucleic acid sequence or a change in a BRCA1 polypeptide sequence (e.g., C61G and M1775R) relative to a normal (e.g., wild type) BRCA1 sequence.

The term “glutathione peroxidase 4” or “GPX4,” as used herein refers to an enzyme encoded by a GPX4 gene. GPX4 is a phospholipid hydroperoxidase that protects cells against membrane lipid peroxidation. Within the meaning of this term, GPX4 encompasses all proteins encoded by a GPX4 gene, mutants thereof, and alternatively spliced proteins thereof. Additionally, as used herein, the term “GPX4” includes GPX4 analogues, homologues and orthologues in other animals.

The term “GPX4 inhibitor” or “GPX4i” refers to any agent that is capable of interacting with GPX4 and inhibits the activity or function of GPX4. The term “GPX4i” or “GPX4 inhibitor” encompasses molecules, including antibodies, peptides, and small molecules that may bind to and inhibit the activity or function of GPX4. The term “GPX4i” or “GPX4 inhibitor” also encompasses any molecule that indirectly inhibits the activity of GPX4. Exemplary molecules are described elsewhere herein.

The term “PARP,” as used herein refers to “Poly (ADP-ribose) polymerase.” The PARP family comprises 17 members which have different structures and functions in a cell. Within the meaning of this term, PARP encompasses all proteins encoded by a PARP gene, mutants thereof, and alternatively spliced proteins thereof. Additionally, as used herein, the term “PARP” includes PARP analogues, homologues and orthologues in other animals. The term “PARP,” includes but is not limited to PARP-1. Additionally, within the meaning of this term PARP-2, PARP-3, Vault-PARP (PARP-4), PARP-7 (TiPARP), PARP-8, PARP-9 (Bal), PARP-10, PARP-11, PARP-12, PARP-13, PARP-14, PARP-15, PARP-16, TANK-1, TANK-2, and TANK-3 may be encompassed.

The term “PARP inhibitor” or “PARPi” refers to molecule that is capable of interacting with a PARP and inhibiting its activity, more particularly its enzymatic activity. Inhibiting PARP enzymatic activity means reducing the ability of a PARP to produce poly(ADP-ribose) or to induce poly(ADP-ribosyl)ation of a substrate. The term “PARP inhibitor” or “PARPi” encompasses molecules, including antibodies, peptides, and small molecules that may bind to and inhibit the function of PARP. Preferably, such inhibition is specific, i.e. the PARP inhibitor reduces the ability of a PARP to produce poly(ADP-ribose) or to induce poly(ADP-ribosyl)ation of a substrate at a concentration that is lower than the concentration of the inhibitor that is required to produce some other, unrelated biological effect. The term “PARP inhibitor” or “PARPi” also encompasses any molecule that indirectly inhibits the activity of PARP.

The term “ferroptosis” refers to regulated cell death that is iron-dependent. Ferroptosis is characterized by the overwhelming, iron-dependent accumulation of lethal lipid reactive oxygen species. Ferroptosis is distinct from apoptosis, necrosis, and autophagy. Ferroptosis induction is determined by methods that are known in the art. See e.g., Dixon, S J, et al., Cell 149, 1060-72 (2012).

As used herein, the term “subject” includes any human or nonhuman animal. The terms, “subject” and “patient” are used interchangeably herein. The term “nonhuman animal” includes, but is not limited to, vertebrates such as dogs, cats, horses, cows, pigs, boar, sheep, goat, buffalo, bison, llama, deer, elk and other large animals, as well as their young, including calves and lambs, and to mice, rats, rabbits, guinea pigs, primates such as monkeys and other experimental animals. Within animals, mammals are preferred, most preferably, valued and valuable animals such as domestic pets, race horses and animals used to directly produce (e.g., meat) or indirectly produce (e.g., milk) food for human consumption, although experimental animals are also included. In specific aspects, the subject is a human. Thus, the present disclosure is applicable to clinical, veterinary and research uses.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated.

As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a GPX4 inhibitor and a PARP inhibitor to an individual in need of such treatment.

The term “therapeutically effective amount” or “effective dose” as used herein refers to an amount of the active ingredient(s) that is (are) sufficient, when administered by a method of the disclosure, to efficaciously deliver the active ingredient(s) for the treatment of a condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; modulate protein methylation in the target cells; and/or relieve, to some extent, one or more of the symptoms associated with the cancer. To the extent the administered compound or composition prevents growth and/or kills existing cancer cells, it may be cytostatic and/or cytotoxic.

In addition, the terms “effective” and “effectiveness” with regard to a treatment disclosed herein includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

The ability of a therapeutic agent to promote disease regression, e.g., cancer regression, can be evaluated using a variety of methods known to the skilled practitioner, such as in human subjects during clinical trials, in animal model systems predictive of efficacy in humans, or by assaying the activity of the agent in in vitro assays.

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

### II. Therapeutic Methods

One aspect of the present disclosure is directed to a method of treating a cancer in a subject in need thereof, comprising administering to the subject a GPX4 inhibitor and a PARP inhibitor, wherein the subject is BRCA1 deficient.

Another aspect of the present disclosure is directed to a method of treating a cancer in a subject in need thereof, comprising administering to the subject a GPX4 inhibitor and a PARP inhibitor, wherein the subject is BRCA1 deficient and the cancer is PARP inhibitor resistant.

Any appropriate method can be used to determine whether or not a cancer has reduced or eliminated BRCA1 polypeptide expression and/or BRCA1 polypeptide activity. For example, the presence, absence, level, or activity of BRCA1 polypeptides can be detected in a sample (e.g., a tumor sample such as a cancer biopsy) obtained from a subject to determine if the subject has a BRCA1-deficient cancer. For example, western blotting, reverse-transcription polymerase chain reaction (RT-PCR), spectrometry methods (e.g., high-performance liquid chromatography (HPLC) and liquid chromatography-mass spectrometry (LC/MS)), enzyme-linked immunosorbent assay (ELISA), and the ability of BRCA1 polypeptides to bind with nucleic acid (e.g., deoxyribonucleic acid (DNA)) can be used to determine whether or not a sample contains a reduced or eliminated level of BRCA1 polypeptide expression or BRCA1 polypeptide activity. In some aspects, when reduced or eliminated BRCA1 polypeptide expression and/or reduced or eliminated BRCA1 polypeptide activity is/are detected in a sample obtained from a subject having cancer, the subject can be identified as being BRCA1 deficient.

In some aspects, the subject has a BRCA1 mutation. Any appropriate method can be used to identify the presence or absence of a mutation in a BRCA1 nucleic acid and/or a BRCA1 polypeptide. In some cases, one or more sequencing techniques (e.g., nucleic acid sequencing techniques or polypeptide sequencing techniques) can be used to identify the presence or absence of a mutation in a BRCA1 nucleic acid and/or a BRCA1 polypeptide. Examples of mutations in a BRCA1 nucleic acid and/or a BRCA1 polypeptide that can alter cancer cell metabolism include, without limitation, epigenetic silencing of BRCA1 (e.g., due to promoter methylation), genomic deletions that include all or part of a BRCA1 nucleic acid, modifications that introduce premature stop codons (e.g., frameshift and nonsense mutations), modifications that alter the coding sequence (e.g., missense mutations), and modifications that lead to truncated BRCA1 polypeptides. In some aspects, a BRCA1-deficient cancer can include one or more cancer cells having one or more modifications. Such modifications include, but are not limited to, a missense mutation such as BRCA1 C64G and M1775R as well as a BRCA1 nonsense or truncating mutation.

In some aspects, the subject does not have BRCA2 deficiency. In some aspects, the subject does not have a BRCA2 mutation. Thus, for example, a subject treatable according to the methods described herein may be BRCA1 deficient but BRCA2 positive or have a wild-type BRCA2 gene.

In some aspects, the cancer in the subject in need thereof is a tumor. In a further aspect, the tumor is a carcinoma. In some aspects, the tumor is a solid tumor. A “solid tumor” includes, but is not limited to, a sarcoma, melanoma, carcinoma, or other solid tumor cancer.

The term “sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzkycell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated according to the methods disclosed herein include, e.g., leukemia, Hodgkin's disease, non-Hodgkin's lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, pancreatic cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, peritoneal cancer, fallopian tube cancer, or uterine papillary serous carcinoma.

In some aspects, the cancer is relapsed, refractory, or refractory following at least one prior therapy comprising administration of at least one anti-cancer agent. The term “relapsed” refers to a situation where a subject, that has had a remission of cancer after a therapy, has a return of cancer cells. As used herein, the term “refractory” or “resistant” refers to a circumstance where a subject, even after intensive treatment, has residual cancer cells in the body. In some aspects, the cancer is metastatic.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, the cancer is selected from the group consisting of breast cancer, ovarian cancer, colon cancer, pancreatic cancer, and prostate cancer. In some aspects, the cancer is breast cancer.

A “cancer” or “cancer tissue” can include a tumor at various stages. In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In other aspects, the cancer or tumor is stage II or stage III, such that, e.g., the cancer or tumor is larger than in stage 0 or stage I, and it has grown into neighboring tissues but it has not metastasized, except potentially to the lymph nodes. In other aspects, the cancer or tumor is stage IV, such that, e.g., the cancer or tumor has metastasized. Stage IV can also be referred to as advanced or metastatic cancer.

GPX4 inhibitors have been disclosed as agents to treat cancer either alone, or with other agents. See, for example, WO/2021/041539A3, WO/2021/041536A1, WO/2021/183702A1, US20210244715, WO/2021/132592A1, US20210380988, US20200299283, U.S. Pat. No. 9,695,133, each of which are herein incorporated by reference in their entirety.

In some aspects, the GPX inhibitor is a class II ferroptosis-inducing compound (FIN) selected from a group consisting of RSL3, (1S,3R)-RSL3, ML162 (DPI7), ML210 (DPI10), CIL56, DPI19, DPI18, DPI17, DPI13, DPI12, altretamine, JKE-1674, JKE1716, AND PACMA 31.

In some aspects, the GPX inhibitor is a class III FIN such as FIN56.

In some aspects, the GPX inhibitor is a class IV FIN such as withaferin A.

In some aspects, the GPX inhibitor is not a class I FIN.

In some aspects, the GPX4 inhibitor is selected from the group consisting of RSL3, ML162, ML210, JKE-1674, withaferin A, and any combination thereof. In aspects, the GPX4 inhibitor is RSL3. In aspects, the GPX4 inhibitor is ML162. In aspects, the GPX4 inhibitor is ML210. In aspects, the GPX4 inhibitor is JKE-1674. In aspects, the GPX4 inhibitor is withaferin A (WFA).

PARP inhibitors have been disclosed as agents for treating cancer either alone, or with other agents. See, for example, US20220047567, US2021025290, WO/2021/119523A1, WO/2020/053125A1, WO/2019/231220A1, WO2016/019125, WO2015/069851, WO2012/054698, WO2010/017055, US20140235675, U.S. Pat. Nos. 1,162,095, 10,563,197 925,580, 9,150,540, 8,946,221, 8,778,966, 8,623,884, 8,623,872, 8,404,713, 8,299,256, 8,188,103, 7,803,795, each of which are herein incorporated by reference in their entirety.

Examples of PARP inhibitors include, but are not limited to olaparib, niraparib, rucaparib, talazoparib, veliparib, iniparib, cediranib, fluzoparib, BGB-290, rucaparib, cediranib, 2X-121, AZD2281, BSI-201, CEP-9722, or a pharmaceutically acceptable salt thereof.

In some aspects, the PARP inhibitor is a PARP-1 or PARP-2 inhibitor. In some aspects, the PARP inhibitor is selected from the group consisting of olaparib, niraparib, veliparib, talazoparib, rucaparib, and fluzoparib. In some aspects, the PARP inhibitor is olaparib. In some aspects, the PARP inhibitor is niraparib. In some aspects, the PARP inhibitor is veliparib. In some aspects, the PARP inhibitor is talazoparib. In some aspects, the PARP inhibitor is rucaparib. In some aspects, the PARP inhibitor is fluzoparib. The PARP inhibitor may, for example, include olaparib, niraparib, rucaparib, talazoparib, or any combination thereof.

In some aspects, the GPX4 inhibitor used herein is RSL3, and the PARP inhibitor used herein is olaparib. In some aspects, the GPX4 inhibitor used herein is ML162, and the PARP inhibitor used herein is olaparib. In some aspects, the GPX4 inhibitor used herein is MLE210, and the PARP inhibitor used herein is olaparib. In some aspects, the GPX4 inhibitor used herein is JKE-1674, and the PARP inhibitor used herein is olaparib. In some aspects, the GPX4 inhibitor used herein is withaferin A, and the PARP inhibitor used herein is olaparib.

In some aspects, the GPX4 inhibitor used herein is RSL3, and the PARP inhibitor used herein is niraparib. In some aspects, the GPX4 inhibitor used herein is ML162, and the PARP inhibitor used herein is niraparib. In some aspects, the GPX4 inhibitor used herein is MLE210, and the PARP inhibitor used herein is niraparib. In some aspects, the GPX4 inhibitor used herein is JKE-1674, and the PARP inhibitor used herein is niraparib. In some aspects, the GPX4 inhibitor used herein is withaferin A, and the PARP inhibitor used herein is niraparib.

In some aspects, the GPX4 inhibitor used herein is RSL3, and the PARP inhibitor used herein is veliparib. In some aspects, the GPX4 inhibitor used herein is ML162, and the PARP inhibitor used herein is veliparib. In some aspects, the GPX4 inhibitor used herein is MLE210, and the PARP inhibitor used herein is veliparib. In some aspects, the GPX4 inhibitor used herein is JKE-1674, and the PARP inhibitor used herein is veliparib. In some aspects, the GPX4 inhibitor used herein is withaferin A, and the PARP inhibitor used herein is veliparib.

In some aspects, the GPX4 inhibitor used herein is RSL3, and the PARP inhibitor used herein is talazoparib. In some aspects, the GPX4 inhibitor used herein is ML162, and the PARP inhibitor used herein is talazoparib. In some aspects, the GPX4 inhibitor used herein is MLE210, and the PARP inhibitor used herein is talazoparib. In some aspects, the GPX4 inhibitor used herein is JKE-1674, and the PARP inhibitor used herein is talazoparib. In some aspects, the GPX4 inhibitor used herein is withaferin A, and the PARP inhibitor used herein is talazoparib.

In some aspects, the GPX4 inhibitor used herein is RSL3, and the PARP inhibitor used herein is rucaparib. In some aspects, the GPX4 inhibitor used herein is ML162, and the PARP inhibitor used herein is rucaparib. In some aspects, the GPX4 inhibitor used herein is MLE210, and the PARP inhibitor used herein is rucaparib. In some aspects, the GPX4 inhibitor used herein is JKE-1674, and the PARP inhibitor used herein is rucaparib. In some aspects, the GPX4 inhibitor used herein is withaferin A, and the PARP inhibitor used herein is rucaparib.

In some aspects, the GPX4 inhibitor used herein is RSL3, and the PARP inhibitor used herein is fluzoparib. In some aspects, the GPX4 inhibitor used herein is ML162, and the PARP inhibitor used herein is fluzoparib. In some aspects, the GPX4 inhibitor used herein is MLE210, and the PARP inhibitor used herein is fluzoparib. In some aspects, the GPX4 inhibitor used herein is JKE-1674, and the PARP inhibitor used herein is fluzoparib. In some aspects, the GPX4 inhibitor used herein is withaferin A, and the PARP inhibitor used herein is fluzoparib.

In some aspects, the GPX4 inhibitor may be provided in an effective amount including about 0.1 mg/day-1200 mg/day, such as about 0.100 mg/day-600 mg/day, or about 0.25 mg/day-1 mg/day. Exemplary effective amounts include about 0.1 mg, 0.25 mg, 0.5 mg, 0.75 mg, 1.0 mg, 100 mg, 200 mg, 300 mg, 400 mg, 500 mg, 600 mg, 700 mg, 750 mg, 800 mg, 900 mg, 1000 mg, 2000 mg, 3000 mg, 4000 mg, 5000 mg, 6000 mg, 7000 mg, and 8000 mg, taken orally in one or two doses per day. A physician prescribing a GPX4 inhibitor to a subject or treating a subject with a GPX4 inhibitor could readily consult resources such as drug product prescribing information or other references (such as the Physicians' Desk Reference) to dose an effective amount of a GPX4 inhibitor.

In some aspects, the PARP inhibitor may be provided in an effective amount including about 0.1 mg/day-1200 mg/day, such as about 0.100 mg/day-600 mg/day, or about 0.25 mg/day-1 mg/day. Exemplary effective amounts include about 0.1 mg, 0.25 mg, 0.5 mg, 0.75 mg, 1.0 mg, 100 mg, 200 mg, 300 mg, 400 mg, 500 mg, 600 mg, 700 mg, 750 mg, 800 mg, 900 mg, and 1000 mg, taken orally in one or two doses per day. A physician prescribing a PARP inhibitor to a subject or treating a subject with a PARP inhibitor could readily consult resources such as drug product prescribing information or other medical or clinical references (such as the Physicians' Desk Reference) to dose an effective amount of a PARP inhibitor.

In some aspects of the methods described herein, the subject is a human.

In some aspects of the methods described herein, a GPX4 inhibitor and a PARP inhibitor are administered to a subject in need thereof simultaneously. In other aspects, a GPX4 inhibitor and a PARP inhibitor are administered to a subject in need thereof sequentially, where, for example, a GPX4 inhibitor is administered before a PARP inhibitor or a GPX4 inhibitor is administered after a PARP inhibitor. When a GPX4 inhibitor and a PARP inhibitor are administered sequentially, the interval or duration of time between administration of each inhibitor can be on the order of minutes, hours, days, or weeks. For example, a GPX4 inhibitor and a PARP inhibitor may be administered within about 1, 5, 10, 20, 30, 40, 50, or 60 minutes of each other, within about 1, 2, 3, 4, 5, 6, 7, 8, 9, 10, 11, 12, 13, 14, 15, 16, 17, 18, 19, 20, 21, 22, 23, or 24 hours of each other, within about 1, 2, 3, 4, 5, 6, or 7 days of each other, or within about 1, 2, 3, 4, 5, 6, 7, 8, 9, or 10 weeks of each other. Selection of the dosing interval between a GPX4 inhibitor and a PARP inhibitor may be made according to the dosing schedules for each inhibitor by consulting a resource such as drug product prescribing information or other medical or clinical references (such as the Physicians' Desk Reference).

The methods described herein can also include additional steps such as prescribing, initiating, and/or altering prophylaxis and/or treatment, based at least in part on the determination of the GPX4, PARP, or both GPX4 and PARP expression levels. In some aspects, the methods disclosed herein further comprise (a) administering chemotherapy; (b) performing surgery; (c) administering radiation therapy; or (d) any combination thereof.

In other aspects, the methods described herein can be combined with standard of care for cancer therapy. In some aspects, standard of care includes, but is not limited to, chemotherapy, radiotherapy, administering immunotherapy, administering targeted therapy, and combination thereof.

In some aspects of the methods described herein, the administration of a GPX4 inhibitor and a PARP inhibitor induces ferroptosis. In some aspects, the administration induces cancer cell death and/or reduces cancer cell growth in the subject.

In some aspects of the methods described herein, the methods disclosed herein reduce the cancer burden.

In some aspects of the methods described herein, the cancer burden is reduced by at least about 5%, at least about 10%, at least about 15%, at least about 20%, at least about 30%, at least about 40%, or about 50% as compared to the cancer burden prior to the administration of a GPX4 inhibitor and a PARP inhibitor.

In some aspects of the methods described herein, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration of the GPXs inhibitor and the PARP inhibitor.

In some aspects, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of a GPX4 inhibitor and a PARP inhibitor. The term “stable disease” refers to a diagnosis for the presence of a cancer, however the cancer has been treated and remains in a stable condition, i.e. one that that is not progressive, as determined, e.g., by imaging data and/or best clinical judgment. The term “progressive disease” refers to a diagnosis for the presence of a highly active state of a cancer, i.e., one that has not been treated and is not stable or has been treated and has not responded to therapy, or has been treated and active disease remains, as determined by imaging data and/or best clinical judgment.

“Stable disease” can encompass a (temporary) tumor shrinkage/reduction in tumor volume during the course of the treatment compared to the initial tumor volume at the start of the treatment (i.e. prior to treatment). In this context, “tumor shrinkage” can refer to a reduced volume of the tumor upon treatment compared to the initial volume at the start of (i.e. prior to) the treatment. A tumor volume of, for example, less than 100% (e.g., of from about 99% to about 66% of the initial volume at the start of the treatment) can represent a “stable disease.”

“Stable disease” can alternatively encompass a (temporary) tumor growth/increase in tumor volume during the course of the treatment compared to the initial tumor volume at the start of the treatment (i.e. prior to treatment). In this context, “tumor growth” can refer to an increased volume of the tumor upon treatment inhibitor compared to the initial volume at the start of (i.e. prior to) the treatment. A tumor volume of, for example, more than 100% (e.g. of from about 101% to about 135% of the initial volume, preferably of from about 101% to about 110% of the initial volume at the start of the treatment) can represent a “stable disease.”

The term “stable disease” can include the following aspects. For example, the tumor volume does, for example, either not shrink after treatment (i.e. tumor growth is halted) or it does, for example, shrink at the start of the treatment but does not continue to shrink until the tumor has disappeared (i.e. tumor growth is first reverted but, before the tumor has, for example, less than 65% of the initial volume, the tumor grows again.

In some aspects, the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the GPX4 inhibitor and the PARP inhibitor.

In some aspects, the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of a GPX4 inhibitor and a PARP inhibitor.

The term “response” when used herein can refer to a “tumor shrinkage” or a reduction in the number of tumors, for example, when a cancer has metastasized. The term “response” can also be reflected in a “complete response” or “partial response” of the patients or the tumors. The term “complete response” as used herein can refer to the disappearance of all signs of cancer in response to a specific therapy disclosed herein. The term “complete response” and the term “complete remission” can be used interchangeably herein. For example, a “complete response” can be reflected in the continued shrinkage of the tumor (as shown in the appended example) until the tumor has disappeared. A tumor volume of, for example, 0% compared to the initial tumor volume (100%) at the start of (i.e. prior to) the treatment can represent a “complete response.”

Treatment with a GPX4 inhibitor and a PARP inhibitor as disclosed herein can result in a “partial response” (or partial remission; e.g. a decrease in the size of a tumor, or in the extent of cancer in the body, in response to the treatment). A “partial response” can encompass a (temporary) tumor shrinkage/reduction in tumor volume during the course of the treatment compared to the initial tumor volume at the start of the treatment (i.e. prior to treatment).

In some aspects, administering a GPX4 inhibitor and a PARP inhibitor improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject not receiving the treatment.

In some aspects, administering a GPX4 inhibitor and a PARP inhibitor improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not receiving the treatment.

### III. Pharmaceutical Compositions

A GPX4 inhibitor and a PARP inhibitor can be administered to a subject in the form of a raw chemical without any other components present. In some aspects, a GPX4 inhibitor and a PARP inhibitor can be administered to a subject as part of a pharmaceutical composition containing the compound combined with a suitable pharmaceutically acceptable carrier. Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

Pharmaceutical compositions within the scope of the present disclosure include all compositions where a GPX4 inhibitor and/or a PARP inhibitor are combined with one or more pharmaceutically acceptable carriers. In certain aspects, a GPX4 inhibitor and a PARP inhibitor is present in the composition in an amount that is effective to achieve its intended therapeutic purpose as described herein. While individual needs may vary, a determination of optimal ranges of effective amounts of each compound is within the skill of the art. Typically, a GPX4 inhibitor and a PARP inhibitor, individually or combined, can be administered to a subject, e.g., a human, at a dose of from about 0.0025 to about 1500 mg per kg body weight of the subject, or an equivalent amount of a pharmaceutically acceptable salt or solvate thereof, per day to treat the particular disorder. A useful dose of a GPX4 inhibitor and/or a PARP inhibitor, individually or combined, administered to a subject is from about 0.0025 to about 150 mg per kg body weight of the subject, or an equivalent amount of the pharmaceutically acceptable salt or solvate thereof. For intramuscular injection, the dose is typically about one-half of the oral dose.

A unit dose may comprise from about 0.01 mg to about 1 g of a GPX4 inhibitor and/or a PARP inhibitor, individually or combined, e.g., about 0.01 mg to about 8000 mg, about 0.01 mg to about 7000 mg, about 0.01 mg to about 6000 mg, about 0.01 mg to about 5000 mg, about 0.01 mg to about 2000 mg, about 0.01 mg to about 1000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 250 mg, about 0.01 mg to about 100 mg, 0.01 mg to about 50 mg, e.g., about 0.1 mg to about 10 mg, of the compound. The unit dose can be administered one or more times daily, e.g., as one or more tablets or capsules, each containing from about 0.01 mg to about 1 g of the compound, or an equivalent amount of a pharmaceutically acceptable salt or solvate thereof.

A pharmaceutical composition comprising a GPX4 inhibitor and a PARP inhibitor can be administered to any subject, e.g., a BRCA1-deficient cancer patient in need thereof, that may experience the beneficial effects of the GPX4 inhibitor and the PARP inhibitor. Foremost among such subjects are mammals, e.g., humans and companion animals, although the disclosure is not intended to be so limited. In some aspects, the subject is a human.

In some aspects of the pharmaceutical compositions described herein, a GPX4 inhibitor can be administered at the same time as a PARP inhibitor (e.g., as part of the same pharmaceutical composition or as part of different pharmaceutical compositions). In other aspects, a GPX4 inhibitor can be administered at different times than a PARP inhibitor. In additional aspects, a GPX4 inhibitor and a PARP inhibitor can be administered sequentially. In an aspect, a GPX4 inhibitor can be administered followed by the PARP inhibitor. In another aspect, a GPX4 inhibitor can be administered preceded by a PARP inhibitor.

A pharmaceutical composition of the present disclosure can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

In some aspects, a pharmaceutical composition of the present disclosure can be administered orally. In some aspects, a pharmaceutical composition of the present disclosure can be administered orally and is formulated into tablets, dragees, capsules, or an oral liquid preparation. In some aspects, the oral formulation comprises extruded multiparticulates comprising a GPX4 inhibitor and a PARP inhibitor.

Alternatively, a pharmaceutical composition of the present disclosure can be administered rectally, and is formulated in suppositories.

Alternatively, a pharmaceutical composition of the present disclosure can be administered by injection.

Alternatively, a pharmaceutical composition of the present disclosure can be administered transdermally.

Alternatively, a pharmaceutical composition of the present disclosure can be administered by inhalation or by intranasal or transmucosal administration.

Alternatively, a pharmaceutical composition of the present disclosure can be administered by the intravaginal route.

A pharmaceutical composition of the present disclosure can contain from about 0.01 to 99 percent by weight, e.g., from about 0.25 to 75 percent by weight, of a GPX4 inhibitor and/or a PARP inhibitor, individually or combined, e.g., about 1%, about 5%, about 10%, about 15%, about 20%, about 25%, about 30%, about 35%, about 40%, about 45%, about 50%, about 55%, about 60%, about 65%, about 70%, or about 75% by weight of a GPX4 inhibitor and/or a PARP inhibitor, individually or combined.

A pharmaceutical composition of the present disclosure is manufactured in a manner which itself will be known in view of the instant disclosure, for example, by means of conventional mixing, granulating, dragee-making, dissolving, extrusion, or lyophilizing processes. Thus, pharmaceutical compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which can be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers can be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound can be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

### IV. Methods of Selecting Subjects for Treatment

A PARP inhibitor and GPX4 inhibitor combination therapy as described herein relates to treatment of a subject with BRCA1 deficiency or BRCA1 mutation. The present disclosure also provides a method of selecting a subject afflicted with a cancer as suitable for treatment with a GPX4 inhibitor and a PARP inhibitor comprising identifying the subject as having a BRCA1 deficiency and treating the subject with a GPX4 inhibitor and a PARP inhibitor.

In some aspects, a method of identifying whether the subject has a BRCA1 deficiency comprises obtaining a cancer sample from the subject and analyzing the sample for BRCA1 expression level. Any appropriate method can be used to determine whether or not a cancer has reduced or eliminated BRCA1 polypeptide expression and/or BRCA1 polypeptide activity. For example, the presence, absence, level, or activity of BRCA1 polypeptides can be detected in a sample (e.g., a tumor sample such as a cancer biopsy) obtained from a subject to determine if the subject has a BRCA1-deficient cancer. For example, western blotting, reverse-transcription polymerase chain reaction (RT-PCR), spectrometry methods (e.g., high-performance liquid chromatography (HPLC) and liquid chromatography-mass spectrometry (LC/MS)), enzyme-linked immunosorbent assay (ELISA), and the ability of BRCA1 polypeptides to bind with nucleic acid (e.g., deoxyribonucleic acid (DNA)) can be used to determine whether or not a sample contains a reduced or eliminated level of BRCA1 polypeptide expression or BRCA1 polypeptide activity. In some cases, when reduced or eliminated BRCA1 polypeptide expression and/or reduced or eliminated BRCA1 polypeptide activity is/are detected in a sample obtained from a subject having cancer, the subject can be identified as having a BRCA1-deficient cancer.

Another aspect of the present disclosure is directed to a method of selecting a subject afflicted with a cancer as suitable for treatment with a GPX4 inhibitor and a PARP inhibitor, the method comprising identifying the subject as having a BRCA1 mutation and treating the subject with a GPX4 inhibitor and a PARP inhibitor. In some aspects, the method of identifying whether the subject has a BRCA1 mutation comprises obtaining a biological sample, such as a cancer sample, from the subject and analyzing the BRCA1 mutation status in the sample. Any appropriate method can be used to identify the presence or absence of a mutation in a BRCA1 nucleic acid and/or a BRCA1 polypeptide. In some cases, one or more sequencing techniques (e.g., nucleic acid sequencing techniques or polypeptide sequencing techniques) can be used to identify the presence or absence of a mutation in a BRCA1 nucleic acid and/or a BRCA1 polypeptide.

In some aspects, the cancer sample comprises tumor tissue, intratumoral tissue, a blood sample, bone marrow, or combinations thereof.

In some aspects, the BRCA1-deficient subject does not have BRCA2 deficiency. In some aspects, the BRCA1-deficient subject does not have BRCA2 mutation. Thus, for example, a subject identified according to the methods of selecting a subject for treatment as described herein may be BRCA1 deficient but BRCA2 positive or have a wild-type BRCA2 gene.

In several aspects of the methods described herein, the samples (e.g., cancer samples or biological samples) can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the quantification of the expression level of BRCA1 and/or BRCA2 disclosed herein, e.g., comparisons between the expression level of a control sample and that of a subject; evaluation of the absence or presence of BRCA1 and/or BRCA2; determination of BRCA1 and/or BRCA2 expression level with respect to a certain threshold; determination of BRCA1 and/or BRCA2 mutation status; treatment decisions; or combinations thereof, can be performed by one or more healthcare providers, healthcare benefits providers, and/or clinical laboratories.

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a GPX4 inhibitor and a PARP inhibitor disclosed herein to treat a cancer. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification of a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits provides can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

### V. Methods of Inducing Ferroptosis

Another aspect of the present disclosure is directed to a method of inducing ferroptosis in a BRCA1-deficient cancer cell comprising contacting the cell with a GPX4 inhibitor and a PARP inhibitor. In some aspects, the ferroptosis induction by a combination of a GPX4 inhibitor and a PARP inhibitor is increased compared to use of a GPX4 inhibitor alone or a PARP inhibitor alone.

In some aspects, ferroptosis induced by a GPX4 inhibitor and a PARP inhibitor is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, or at least about 50-fold as compared to use of a GPX4 inhibitor alone or a PARP inhibitor alone.

In some aspects, after inducing ferroptosis in the BRCA1-deficient cancer cell according to the methods described herein, cell death in the cancer cell is increased compared to use of a GPX4 inhibitor alone or a PARP inhibitor alone. Cell death is determined by methods that are well-known in the art, such as described in the Examples herein.

In some aspects, after inducing ferroptosis in the BRCA1-deficient cancer cell according to the methods described herein, cell death is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, or at least about 50-fold as compared to use of a GPX4 inhibitor alone or a PARP inhibitor alone.

In some aspects, after inducing ferroptosis in the BRCA1-deficient cancer cell with the method described herein, cell viability in the cancer cell is reduced comopared to use of a GPX4 inhibitor alone or a PARP inhibitor alone. The cell viability is determined by methods that are well-known in the art. The cell viability was determined by a method of transcriptional and translational assay (MTT assay).

In some aspects, after inducing ferroptosis in the BRCA1-deficient cancer cell with the method described herein, cell viability is reduced by at least about 5%, at least about 10%, at least about 15%, at least about 20%, at least about 25%, at least about 30%, at least about 35%, at least about 40%, at least about 50%, at least about 60%, at least about 70%, at least about 80%, at least about 90%, or at least about 95% as compared to use of a GPX4 inhibitor alone or a PARP inhibitor alone.

In some aspects, after inducing ferroptosis in the BRCA1-deficient cancer cell with the method described herein, lipid peroxidation in the cancer cell is increased comopared to use of a GPX4 inhibitor alone or a PARP inhibitor alone. Lipid peroxidation is determined by methods that are well-known in the art.

In some aspects, after inducing ferroptosis in the BRCA1-deficient cancer cell with the method described herein, cell death is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, or at least about 50-fold as compared to use of a GPX4 inhibitor alone or a PARP inhibitor alone.

In some aspects, the GPX4 inhibitor is selected from the group consisting of RSL3, ML162, ML210, JKE-1674 and withaferin A. In some aspects, the PARP inhibitor is selected from the group consisting of olaparib, niraparib, veliparib, talazoparib, rucaparib, and fluzoparib.

In some aspects, the GPX4 inhibitor used herein is ML162, and the PARP inhibitor used herein is olaparib. In some aspects, the GPX4 inhibitor used herein is MLE210, and the PARP inhibitor used herein is olaparib. In some aspects, the GPX4 inhibitor used herein is JKE-1674, and the PARP inhibitor used herein is olaparib. In some aspects, the GPX4 inhibitor used herein is withaferin A, and the PARP inhibitor used herein is olaparib.

In some aspects, the GPX4 inhibitor used herein is RSL3, and the PARP inhibitor used herein is niraparib. In some aspects, the GPX4 inhibitor used herein is ML162, and the PARP inhibitor used herein is niraparib. In some aspects, the GPX4 inhibitor used herein is MLE210, and the PARP inhibitor used herein is niraparib. In some aspects, the GPX4 inhibitor used herein is JKE-1674, and the PARP inhibitor used herein is niraparib. In some aspects, the GPX4 inhibitor used herein is withaferin A, and the PARP inhibitor used herein is niraparib.

In some aspects, the GPX4 inhibitor used herein is RSL3, and the PARP inhibitor used herein is veliparib. In some aspects, the GPX4 inhibitor used herein is ML162, and the PARP inhibitor used herein is veliparib. In some aspects, the GPX4 inhibitor used herein is ML210, and the PARP inhibitor used herein is veliparib. In some aspects, the GPX4 inhibitor used herein is JKE-1674, and the PARP inhibitor used herein is veliparib. In some aspects, the GPX4 inhibitor used herein is withaferin A, and the PARP inhibitor used herein is veliparib.

In some aspects, the GPX4 inhibitor used herein is RSL3, and the PARP inhibitor used herein is talazoparib. In some aspects, the GPX4 inhibitor used herein is ML162, and the PARP inhibitor used herein is talazoparib. In some aspects, the GPX4 inhibitor used herein is ML210, and the PARP inhibitor used herein is talazoparib. In some aspects, the GPX4 inhibitor used herein is JKE-1674, and the PARP inhibitor used herein is talazoparib. In some aspects, the GPX4 inhibitor used herein is withaferin A, and the PARP inhibitor used herein is talazoparib.

In some aspects, the GPX4 inhibitor used herein is RSL3, and the PARP inhibitor used herein is rucaparib. In some aspects, the GPX4 inhibitor used herein is ML162, and the PARP inhibitor used herein is rucaparib. In some aspects, the GPX4 inhibitor used herein is ML210, and the PARP inhibitor used herein is rucaparib. In some aspects, the GPX4 inhibitor used herein is JKE-1674, and the PARP inhibitor used herein is rucaparib. In some aspects, the GPX4 inhibitor used herein is withaferin A, and the PARP inhibitor used herein is rucaparib.

In some aspects, the GPX4 inhibitor used herein is RSL3, and the PARP inhibitor used herein is fluzoparib. In some aspects, the GPX4 inhibitor used herein is ML162, and the PARP inhibitor used herein is fluzoparib. In some aspects, the GPX4 inhibitor used herein is ML210, and the PARP inhibitor used herein is fluzoparib. In some aspects, the GPX4 inhibitor used herein is JKE-1674, and the PARP inhibitor used herein is fluzoparib. In some aspects, the GPX4 inhibitor used herein is withaferin A, and the PARP inhibitor used herein is fluzoparib.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: BRCA1 Deficiency Promotes Gpx4 Inhibitor-Induced Ferroptosis

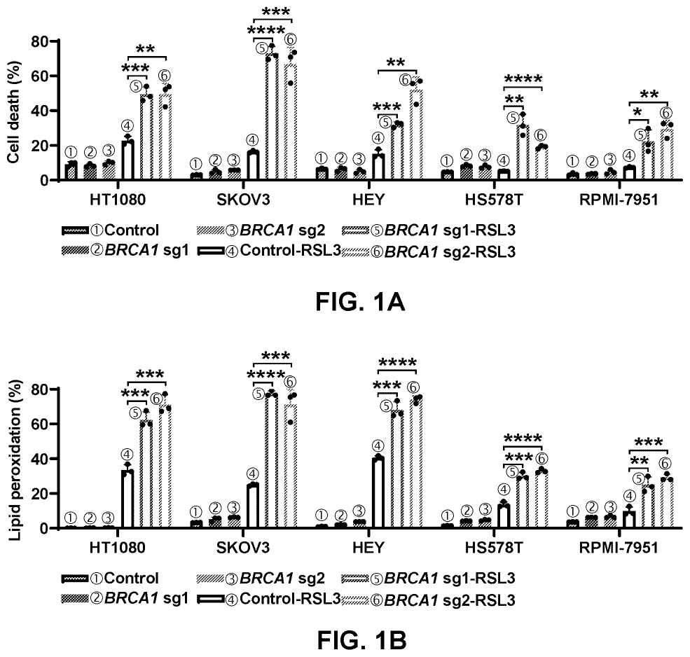

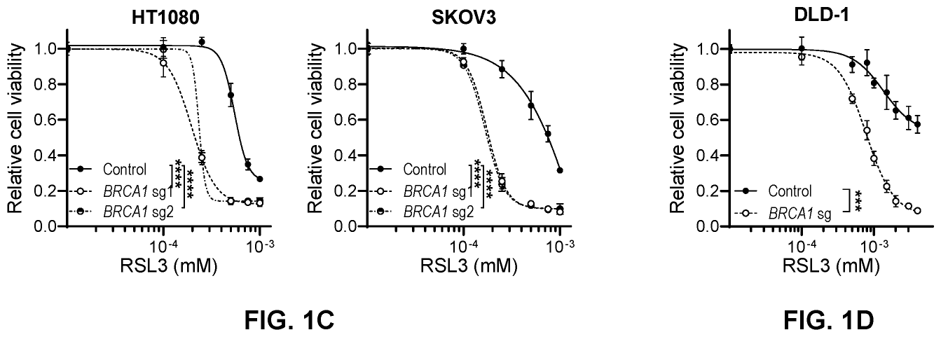

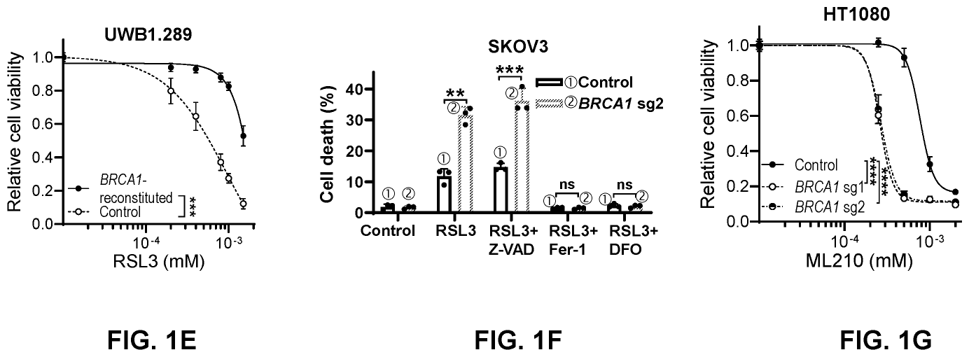

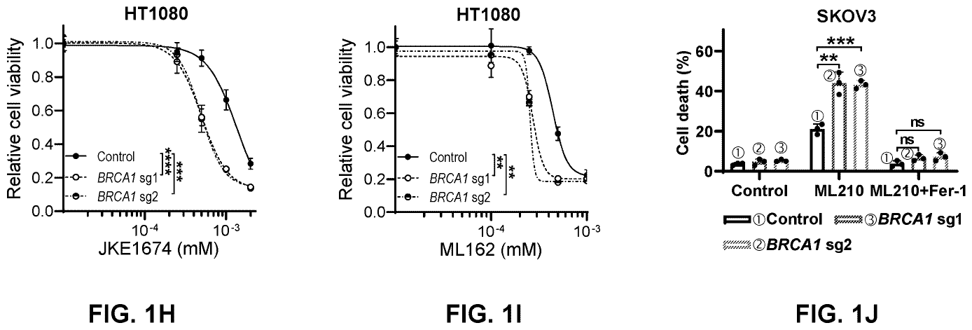

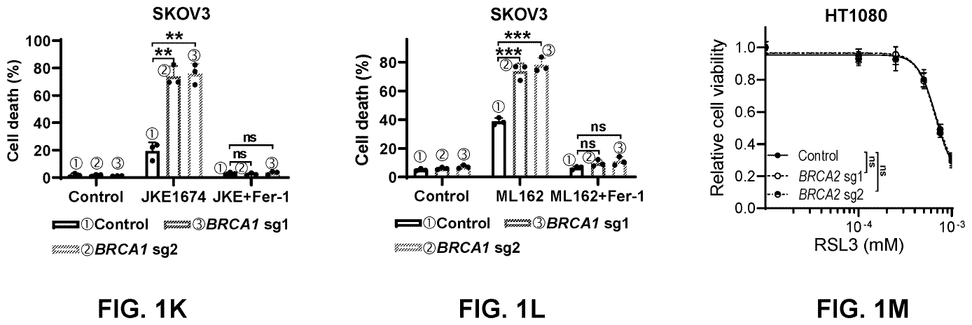

To identify potential ferroptosis vulnerabilities induced by tumor suppressor loss in cancer cells, whether deleting common tumor suppressors rendered cancer cells susceptible to FINs was systematically examined. Decreasing BRCA1 expression in diverse cancer cell lines by the CRISPR/Cas9 approach significantly promoted lipid peroxidation and ferroptosis induced by the GPX4 inhibitor RSL3 (FIGS. 1A-1D and FIGS. 8B-8C). Conversely, restoring BRCA1 expression in the BRCA1-mutant UWB1.289 cells promoted resistance to RSL3-induced ferroptosis (FIG. 1E and FIG. 8C). RSL3-induced cell death in BRCA1 sgRNA-infected (BRCA1 sg) cells, when compared to control, was abolished by the ferroptosis inhibitor ferrostatin-1 or the iron chelator deferoxamine (DFO), but not by the apoptosis inhibitor Z-VAD, confirming that the cell death induced in this context was ferroptosis (FIG. 1F and FIG. 8E). Similar observations were made by using other GPX4 inhibitors, including ML162, ML210, and JKE-1674 (FIGS. 1G-1L). Of note, in these analyses pooled cells infected with BRCA1 sgRNAs were generated, which exhibited residual BRCA1 expression (FIG. 8B) and unaltered cell proliferation rates (FIGS. 8F-8K). Similarly, restoring BRCA1 expression in the BRCA1-mutant UWB1.289 cells did not affect cell proliferation (FIG. 8L). In line with the data with BRCA1 depletion, analyses of data from the Cancer Therapeutics Response Portal (CTRP) [1] revealed that BRCA1 expression correlated with cellular resistance to GPX4 inhibitors (FIG. 1I; it should be noted that correlation scores of GPX4 inhibitor were even higher than those of PARP inhibitor and cisplatin).

Deletion of tumor suppressors invovled in the Hippo pathway, such as E-cadherin (ECAD), renders cancer cells sensitive to ferroptosis [16]. ECAD deletion conferred sensitization to RSL3-induced ferroptosis in HT1080 cells and the phenotypes of sensitization to RSL3-induced ferroptosis were comparable between ECAD-knockout (KO) and BRCA1-KO HT1080 cells (FIG. 1C and FIG. 8M-8N).

BRCA2 is another breast and ovarian cancer susceptibility gene [2]. BRCA1 and BRCA2 form a complex to regulate DNA repair [2], and deficiency in either BRCA1 or BRCA2 renders cancer cells hypersensitive to PARP inhibitor [3, 4]. Therefore, the potential role of BRCA2 in ferroptosis was examined. In contrast to BRCA1 deficiency, BRCA2 depletion did not affect cellular sensitivity to RSL3 (FIG. 1M and FIGS. 8O-8R). Together, these data show that BRCA1-deficient (but not BRCA2-deficient) cancer cells are sensitive to GPX4 inhibitor-induced ferroptosis.

### Example 2: BRCA1 Deficiency Interferes with Gpx4 Transcription

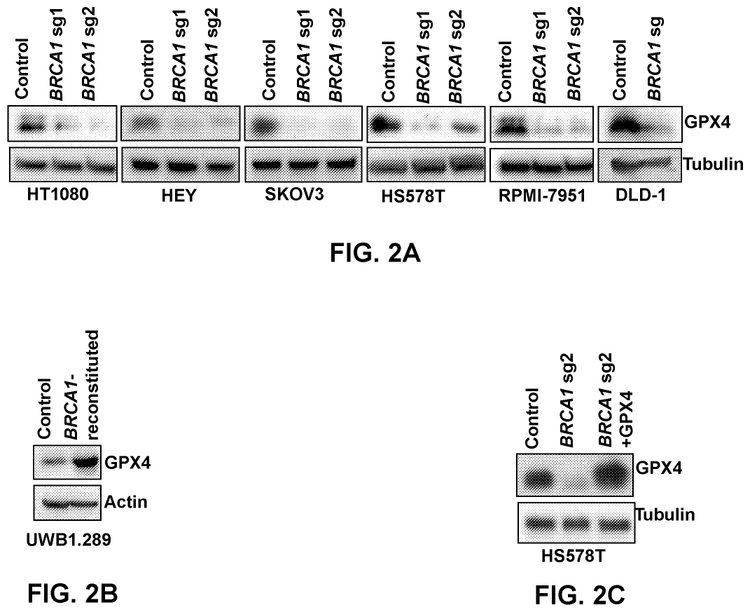

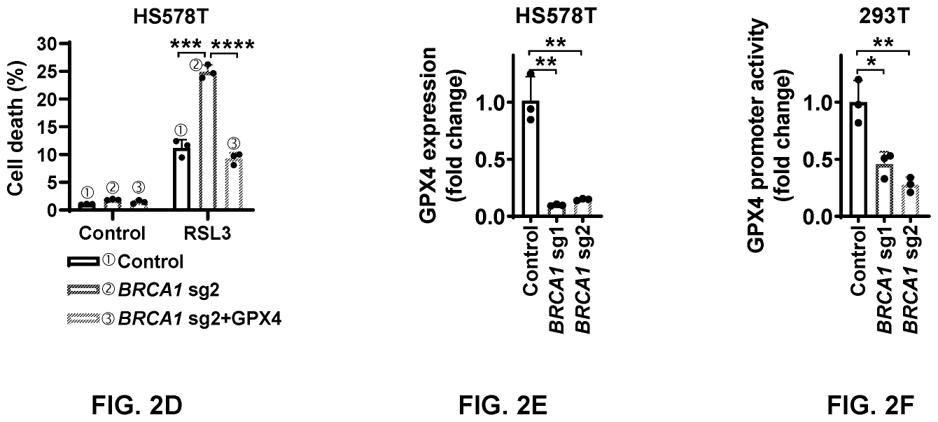

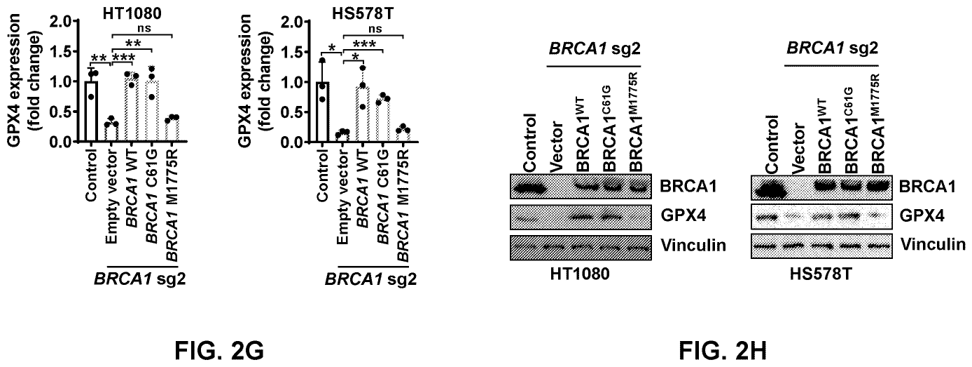

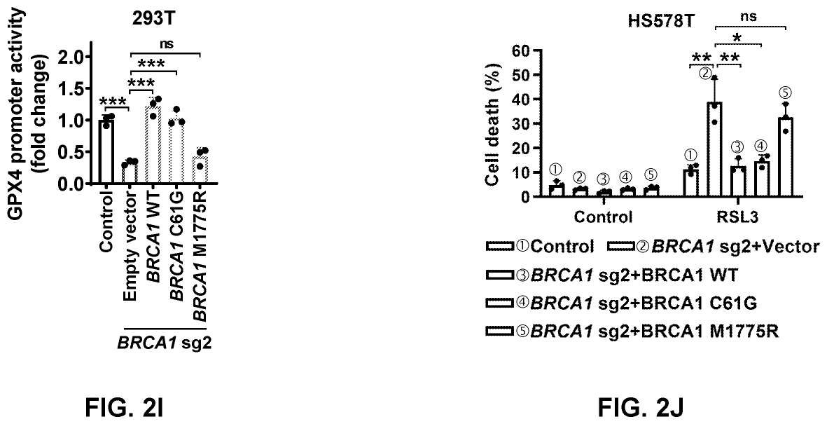

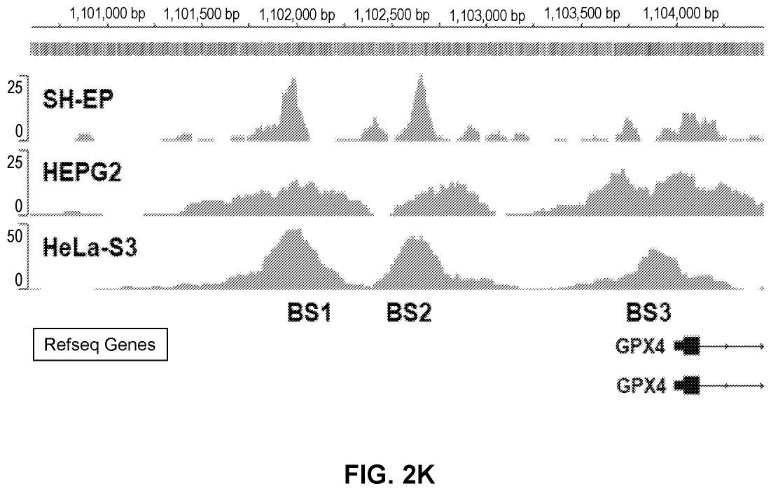

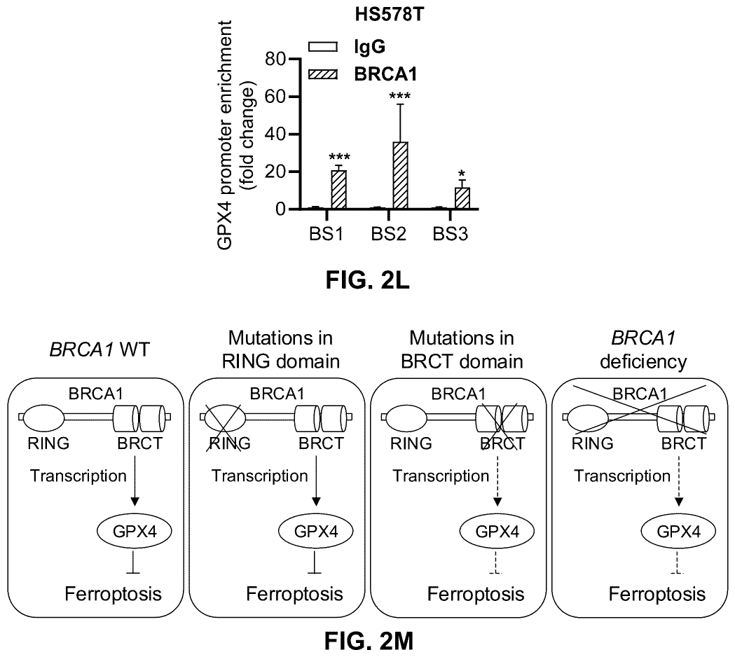

To determine how BRCA1 regulates GPX4i-induced ferroptosis, it was examined whether BRCA1 deficiency affects GPX4 levels or activity, and found that BRCA1 deficiency decreased GPX4 levels (FIG. 2A,) but did not affect the levels of other known ferroptosis regulators, such as ACSL4, FSP1, and DHODH (FIG. 9A). Conversely, BRCA1 restoration in BRCA1-mutant UWB1.289 cells increased GPX4 levels (FIG. 2B, FIGS. 8B and 8B). GPX4 expression in BRCA1-deficient cells suppressed RSL3-induced ferroptosis (FIGS. 2C and 2D). As a comparison, BRCA2 deficiency did not affect expression levels of GPX4 (FIG. 9B). Further analyses revealed that BRCA1 sg cells exhibited decreases in GPX4 mRNA levels (FIG. 2E) and GPX4 promoter luciferase activity (FIG. 2F). Re-expression of BRCA1-wild-type (WT) or BRCA1-C61G mutant (a patient-derived mutant that disrupts its RING domain function), but not BRCA1-M1775R mutant (a patient-derived mutant in the BRCT domain), in BRCA1 sgRNA infected cells restored GPX4 expression levels (FIGS. 2G, 2H, 9C, and 9D) and GPX4 promoter activity (FIG. 2I), and attenuated RSL3-induced ferroptosis (FIG. 2J and FIG. 2M. These data suggest that BRCA1 regulates GPX4 transcription through its BRCT domain.

To further examine how BRCA1 regulates GPX4 transcription, and consistent with BRCA's role in promoter binding and transcriptional regulation [17], analyses of BRCA1 chromatin immunoprecipitation coupled with high-throughput sequencing (ChIP-seq) datasets from GEO revealed several BRCA1 binding sites (BS1-3) on the GPX4 promoter within 3 kb upstream of GPX4 transcription start site in diverse cancer cell lines (FIG. 2K). BRCA1 binding on these sites was further confirmed by ChIP assay in HS578T cells (FIG. 2L). Collectively, BRCA1 deficiency impairs GPX4 transcription and BRCA1's BRCT domain is critical for this regulation, leading to reduced GPX4 expression and increased sensitivity to GPX4i-induced ferroptosis in BRCA1-deficient cells or BRCA1-M1775R mutant-expressing cells (FIG. 2M).

### Example 3: BRCA1 Deficiency Decreases NRF2 Levels and BRCA1 Regulates Gpx4 Transcription Through BRCA1 Interactions with NRF2

To study how BRCA1 regulates GPX4 transcription, a series of GPX4 promoter truncating mutants (FIG. 15A) was generated, and the results show that BRCA1 overexpression-stimulated GPX4 promoter luciferase activity was significantly attenuated in truncating mutant #6, in which GPX4 promoter region at −1,000 to −800 nt upstream of the transcription start site was deleted (FIG. 15B and FIG. 15A). BRCA1 may regulate GPX4 transcription through its interaction with a transcription factor that binds to the GPX4 promoter at the −1,000 to −800 nt region. To test this, a putative transcription factor binding sites in this region was analyzed to investigate whether BRCA1 is shown to interact with such transcription factor(s). These analyses identified an NRF2 binding site (also known as antioxidant response element [ARE]) within the −1,000 to −800 nt region of the GPX4 promoter; importantly, it is known that NRF2 interacts with BRCA1. Mutation of the ARE at the −1,000 to −800 nt region (but not mutation of the ARE identified from another promoter region) abolished BRCA1 overexpression-induced GPX4 promoter activity (FIGS. 15D and 15E).

NRF2 is a transcription factor that governs the transcription of genes involved in antioxidant response. Previous studies have shown that BRCA1 interacts with NRF2 and promotes NRF2-mediated antioxidant-response gene transcription through at least two mechanisms: (i) BRCA1-NRF2 interaction promotes the transcription of NRF2 targets, including NRF2 gene itself and (ii) NRF2 normally is subjected to KEAP1-mediated proteasomal degradation, and BRCA1 interferes with the NRF2-KEAP1 interaction and promotes NRF2 protein stability; consequently, BRCA1 deficiency decreases NRF2 protein levels. The results also suggests reduced NRF2 levels in BRCA1 sgRNA infected cells generated in the studies (FIG. 15F).

Notably, re-expression of BRCA1 WT or C64G mutant, but not its M1775R mutant, in BRCA1-deficient cells also restored NRF2 levels (FIGS. 15G-15L). Furthermore, M1775R mutation, but not C64G mutation, in BRCA1 sg infected cells abolished BRCA1 interaction with NRF2 (FIG. 15P), which corresponded with these mutants' abilities to regulate NRF2 and GPX4 expression or RSL3-induced ferroptosis (FIGS. 2G-2J, 15G, 15L); in contrast, re-expression of either BRCA1 C64G or M1775R mutant restored VDAC3 expression (FIGS. 15N and 15O) and Erastin-induced ferroptosis in BRCA1-deficient cells (FIG. 15M), suggesting that BRCA1 regulates RSL3- or Erastin-induced ferroptosis through different mechanisms. Of note, while NRF2 is known to modulate antioxidant gene transcription, whether GPX4 is a transcriptional target of NRF2 remains unclear; in addition, GPX4 was not studied in previous publications on the BRCA1-NRF2 interaction [8-10]. Analyses by chromatin immunoprecipitation (ChIP) revealed the binding of BRCA1 or NRF2 protein on the GPX4 or NRF2 promoter in control cells but not in BRCA1-depleted cells (FIGS. 15Q and 15V); importantly, re-expression of BRCA1 WT or C64G mutant, but not its M1775R mutant, in BRCA1-depleted cells restored BRCA1 or NRF2 binding on the GPX4 or NRF2 promoter (FIGS. 15Q and 15V). Moreover, BRCA2 deficiency did not affect expression levels of NRF2, GPX4, or VDAC3 (FIG. 15R).

Finally, the data suggests that NRF2 induction by NRF2 inducers (tert-butylhydroquinone and sulforaphane) increased GPX4 levels in BRCA1-mutant SUM149 cells (FIG. 15S). NRF2 deletion, similar to BRCA1 deficiency, reduced GPX4 levels in BRCA1-WT SKOV3 cells (FIG. 15T) and rendered these cells sensitive to RSL3-induced ferroptosis (FIG. 15U); conversely, treatment with tert-butylhydroquinone induced both NRF2 and GPX4 levels in BRCA1 sgRNA infected SKOV3 cells (FIG. 15T) and correspondingly, rendered these cells resistant to RSL3-induced ferroptosis (FIG. 15U). Collectively, the data suggest that BRCA1 deficiency impairs NRF2-mediated GPX4 transcription through interacting with NRF2 via BRCA1's BRCT domain, leading to reduced GPX4 expression and increased sensitivity to GPX4 inhibitor-induced ferroptosis in BRCA1-deficient cells (FIG. 15V).

While inducing lipid peroxidation, PARP inhibitor treatment alone did not trigger strong ferroptosis (although BRCA1 deficiency moderately increased PARP inhibitor-induced ferroptosis; see FIGS. 11D and 11E). It is possible that PARP inhibitor treatment also induces adaptive responses that dampen PARP inhibitor-induced lipid peroxidation and ferroptosis. The results show that PARP inhibitor potently raised NRF2 and GPX4 levels in BRCA1-WT HS578T cells, but not in BRCA1 sg counterparts or BRCA1-mutant HCC1937 or SUM149 cells (FIGS. 16B and 16C). Consistently, PARP inhibitor moderately induced lipid peroxidation in HS578T cells, but induced lipid peroxidation more dramatically in BRCA1-depleted HS578T or BRCA1-mutant HCC1937 or SUM149 cells (FIGS. 11D and 16A). Overexpression of GPX4 in BRCA1-mutant SUM149 cells (which exhibited lower GPX4 levels under PARP inhibitor treatment; see FIG. 16C) suppressed olaparib+RSL3 combination-induced ferroptosis (FIGS. 6V and 16I), whereas GPX4 depletion in HS578T cells (which exhibited higher GPX4 levels under PARP inhibitor treatment; see FIG. 16B) dramatically increased olaparib-induced ferroptosis (FIG. 6T and FIG. 6S). Likewise, NRF2 deletion in HS578T cells not only decreased basal GPX4 levels but also abolished PARP inhibitor-induced GPX4 expression (FIG. 16D). Inducing NRF2 expression by tert-butylhydroquinone treatment in SUM149 cells suppressed, whereas NRF2 deletion in HS578T cells dramatically promoted ferroptosis induced by olaparib+RSL3 combination (FIGS. 16E and 16F; NRF2 blots for tert-butylhydroquinone-treated SUM149 cells and NRF2 HS578T KO cells are shown in FIG. 15S and FIG. 15F, respectively).

How PARP inhibitor induces NRF2 levels was investigated. Previous studies showed that PARP inhibitor triggers reactive oxygen species (ROS) [13], and it is known that oxidative stress stabilizes NRF2 to induce anti-oxidant responses. PARP inhibitor-induced ROS in HS578T cells; interestingly, PARP inhibitor induced even higher levels of ROS in BRCA1 sg HS578T cells or BRCA1-deficient SUM149 cells (FIG. 16G and FIG. 13I), likely because of the decreased NRF2 levels in BRCA1-depleted/-deficient cells. Treatment with the ROS scavenger N-acetyl-1-cysteine (NAC) abolished PARP inhibitor-induced NRF2 and GPX4 induction in BRCA1-WT HS578T cells (FIG. 16H), and partially restored PARP inhibitor-induced cell killing in BRCA1-deficient SUM149 cells (FIG. 13K). Furthermore, the results show that treatment with the ROS inducer tert-butyl hydroperoxide (TBH) (FIGS. 16I and 16J) or PARP inhibitor (FIGS. 13F-13H) induced more cell death in BRCA1-deficient SUM149 cells or BRCA1 sg HS578T cells than in BRCA1-proficient HS578T cells; importantly, the cell death could be largely (for TBH treatment) or partially (for PARP inhibitor treatment) suppressed by the ferroptosis inhibitor treatment, suggesting ferroptosis induction in these contexts.

Together, the data suggest a model that PARP inhibitor on one hand promotes lipid peroxidation through enhancing NCOA4-mediated ferritinophagy, and on the other hand induces GPX4 expression in a ROS-NRF2-BRCA1-dependent manner, which acts as an adaptive response to antagonize PARP inhibitor-induced lipid peroxidation and ferroptosis (FIG. 16K). Consequently, GPX4 inhibition or BRCA1 deficiency alone partially attenuates this adaptive response, resulting in moderate ferroptosis (FIGS. 16L and 16M), whereas combining GPX4 inhibition and BRCA1 deficiency abrogates this adaptive response, leading to potent ferroptosis induction by PARP inhibitor+GPX4 inhibitor treatment in BRCA1-deficient cancer cells (FIG. 16N).

### Example 4: Brca1 Deficiency Suppresses Erastin-Induced Ferroptosis Via Interference with Vdac3 Transcription and Mitochondrial Lipid Peroxidation

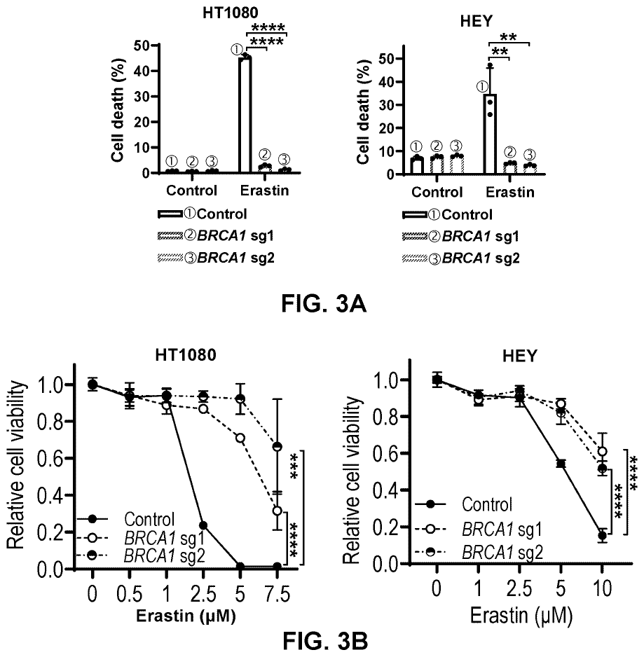

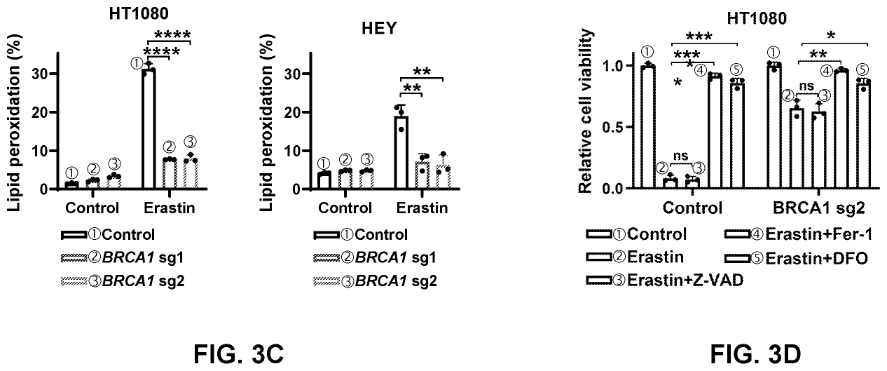

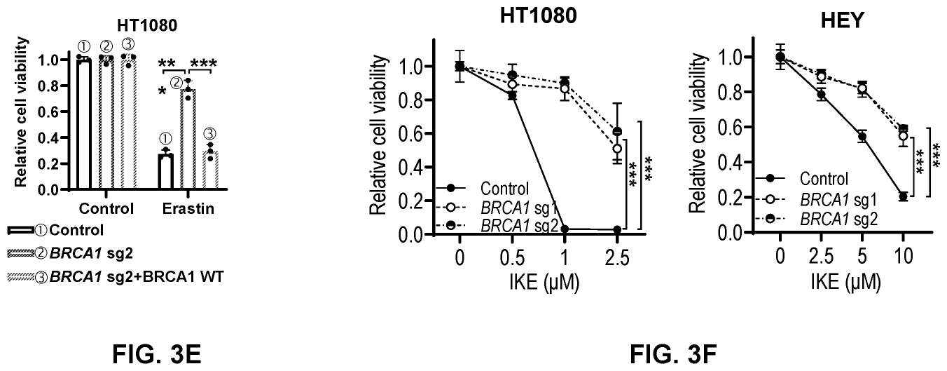

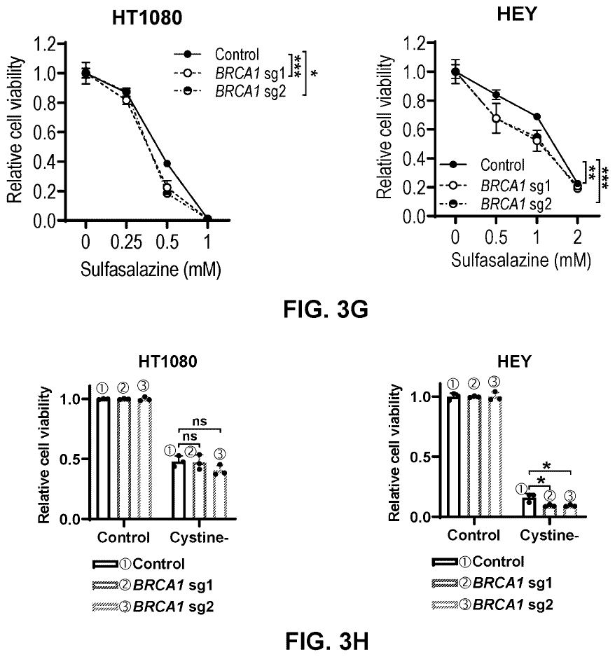

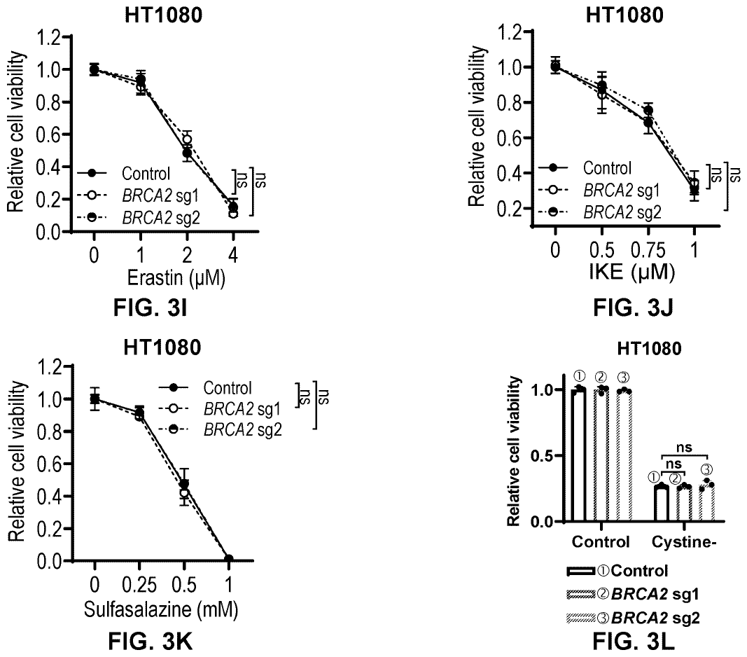

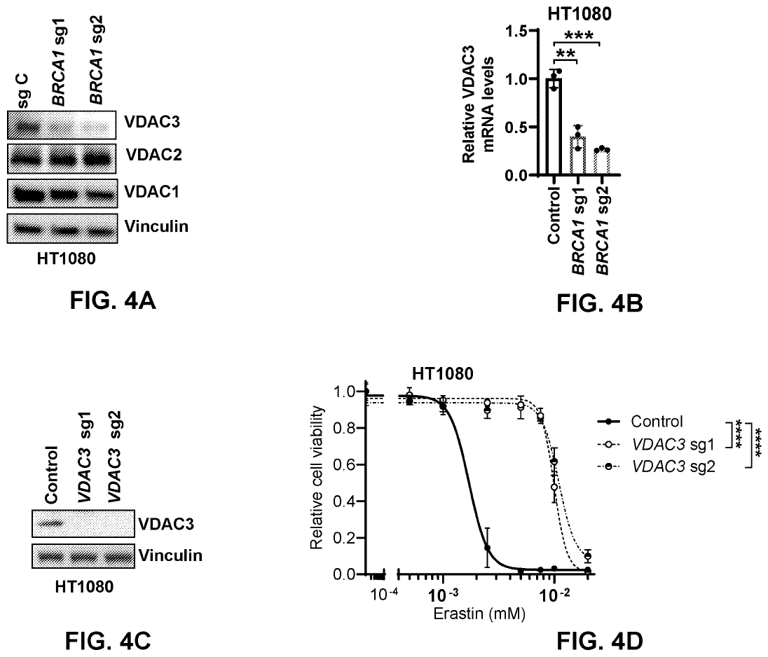

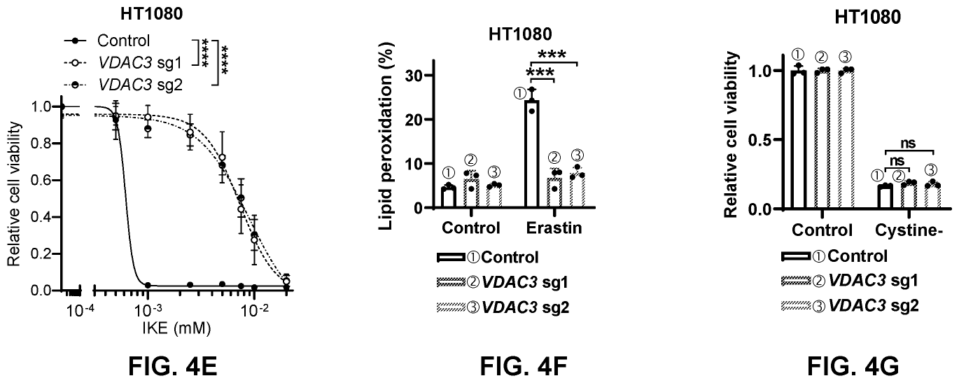

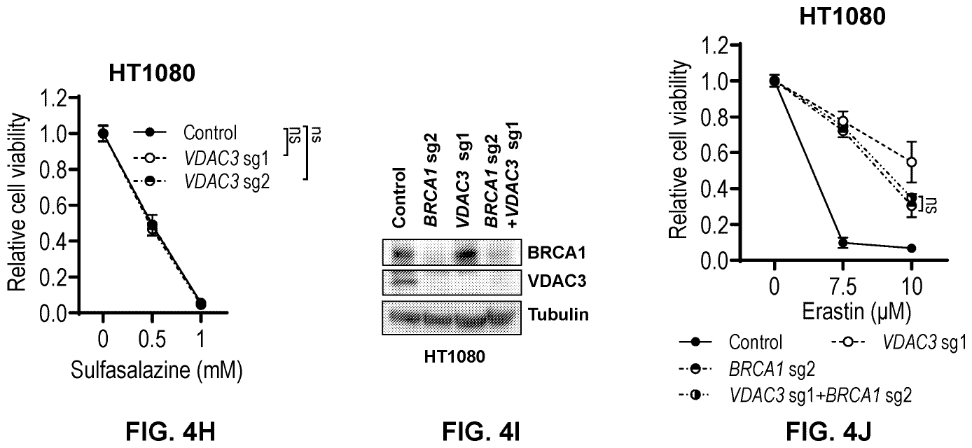

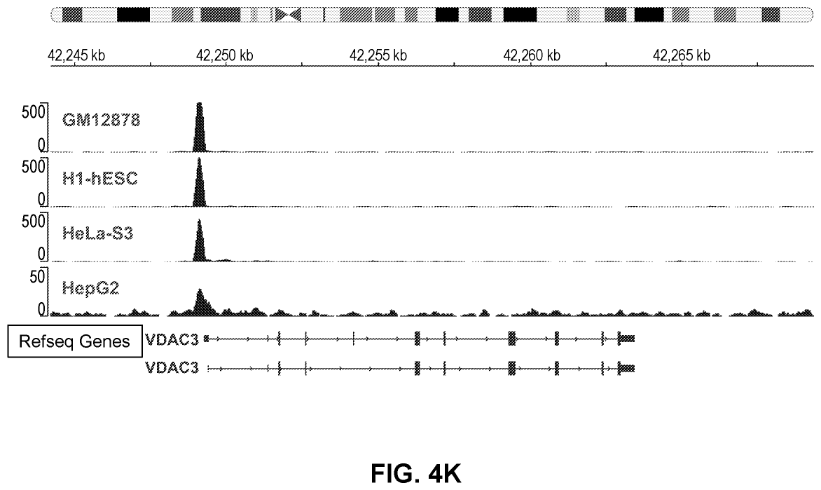

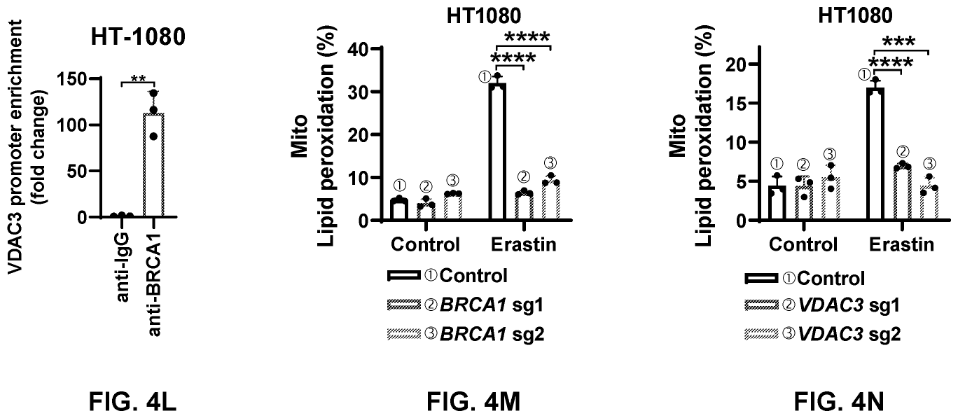

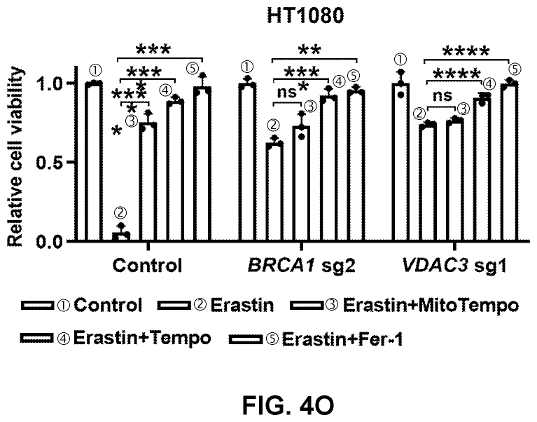

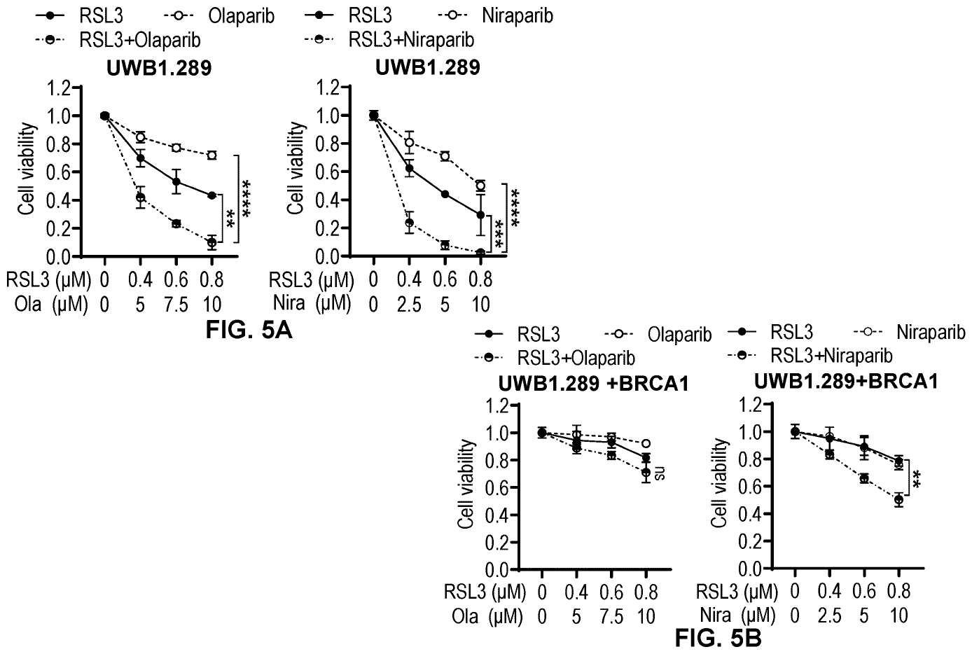

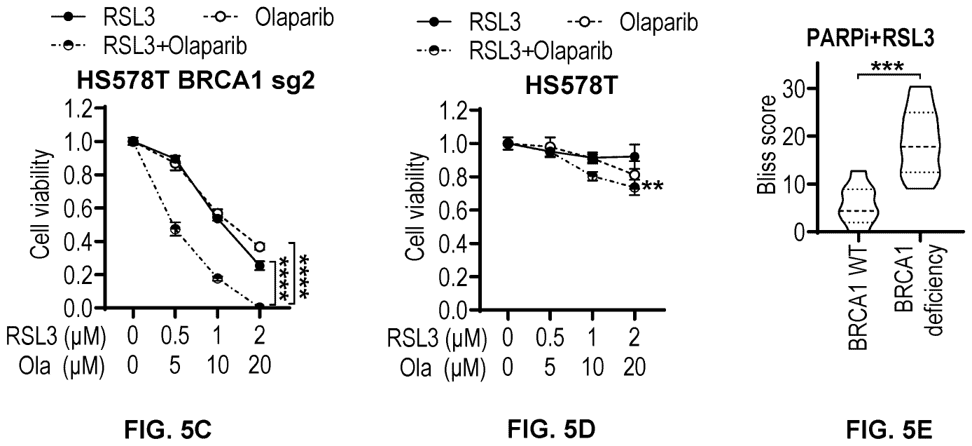

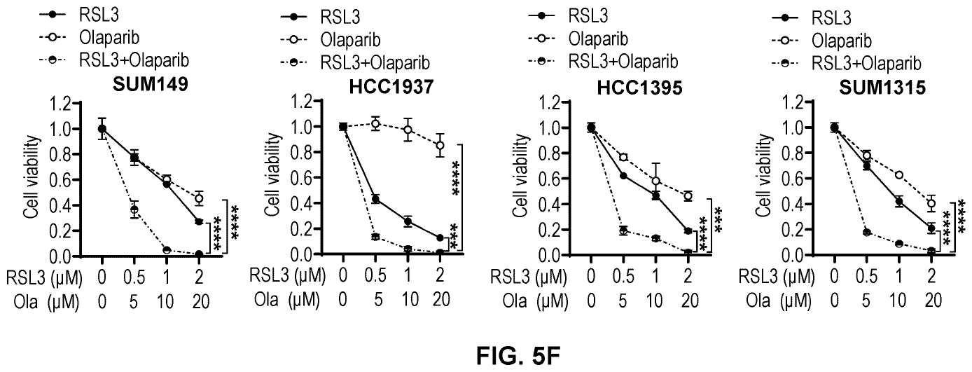

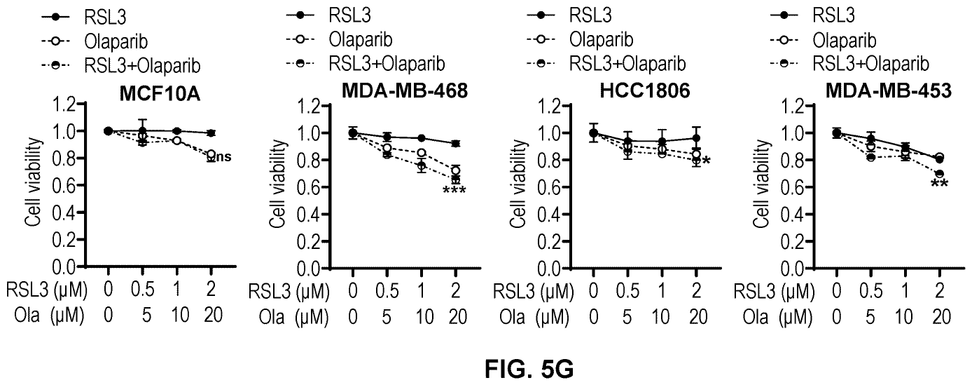

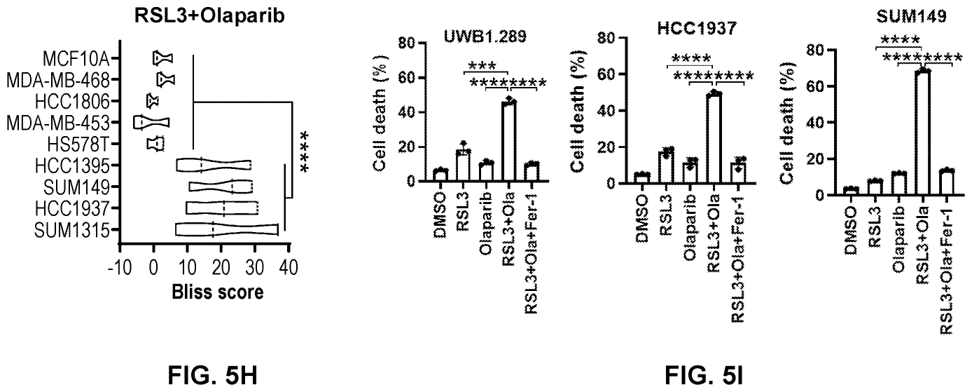

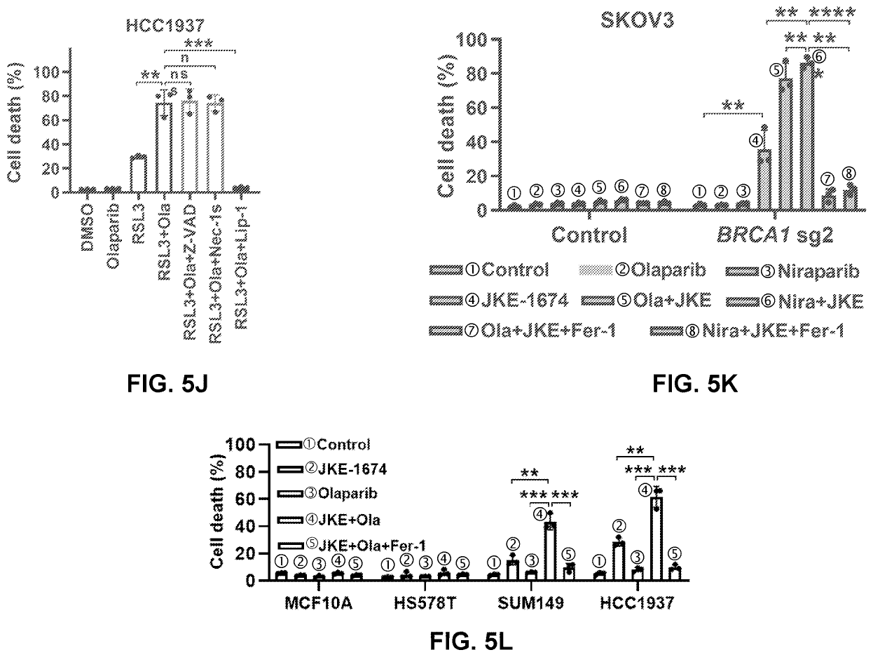

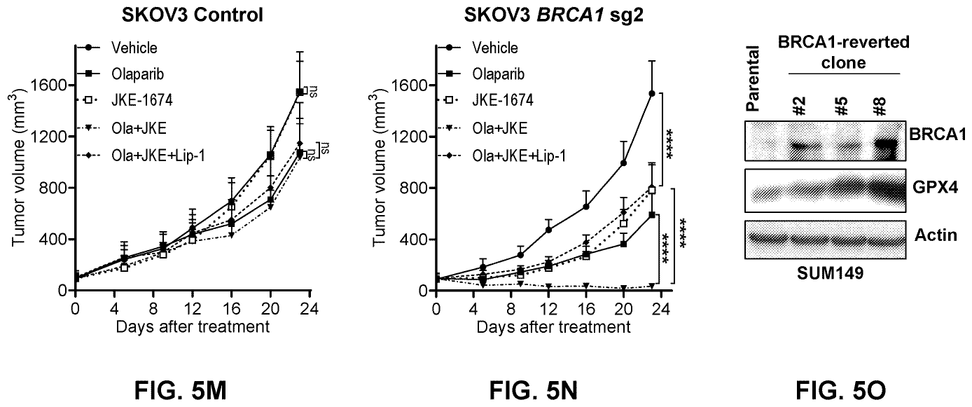

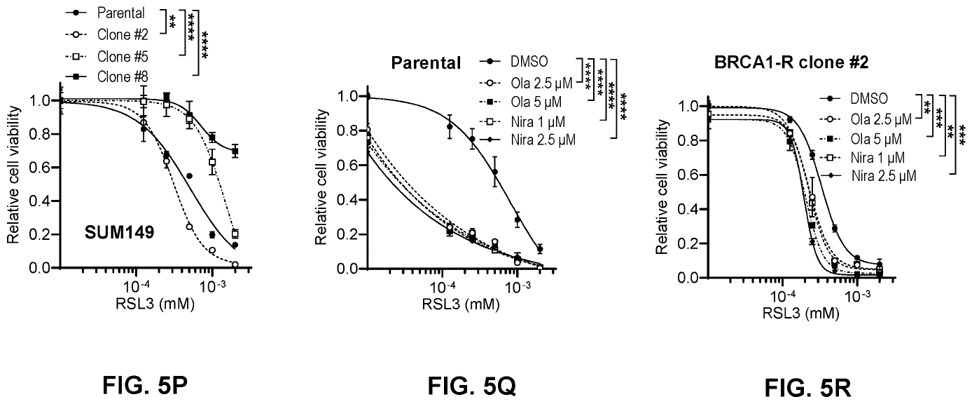

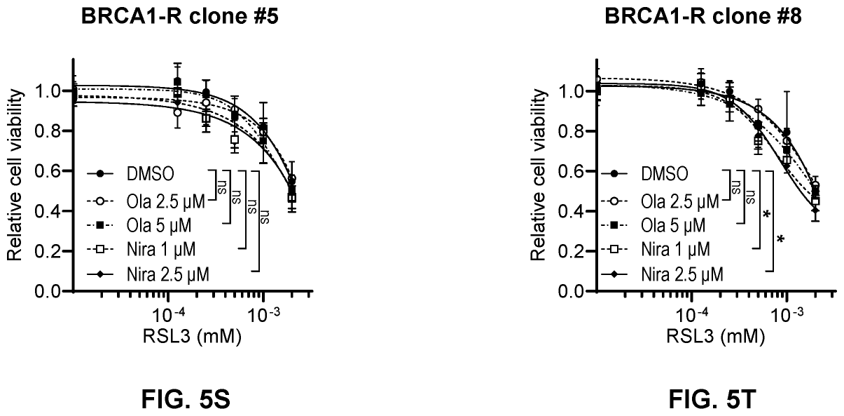

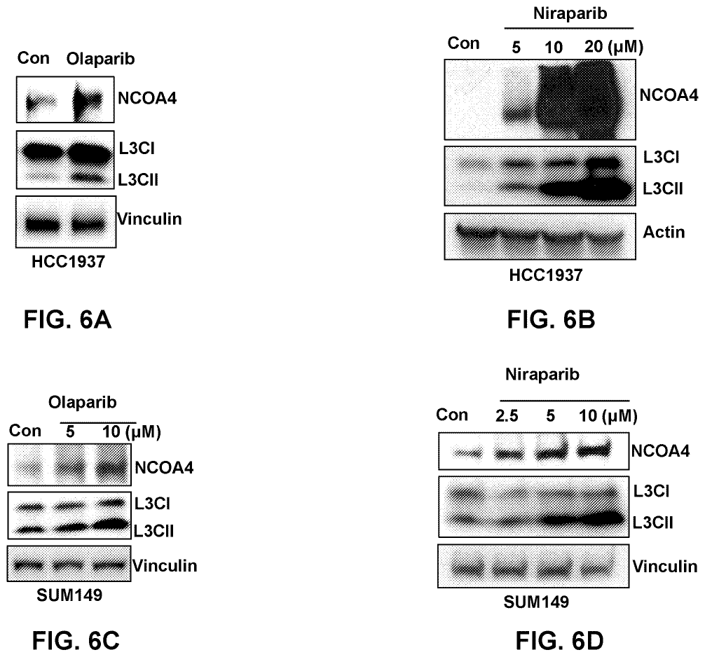

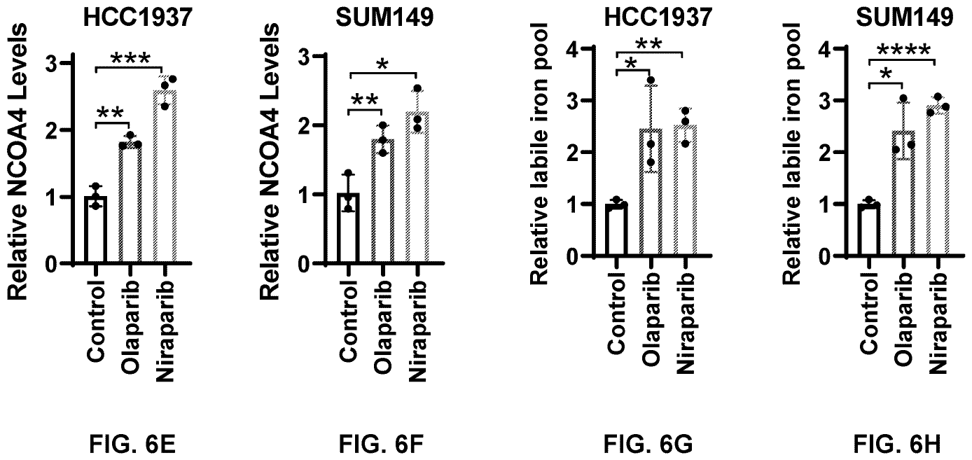

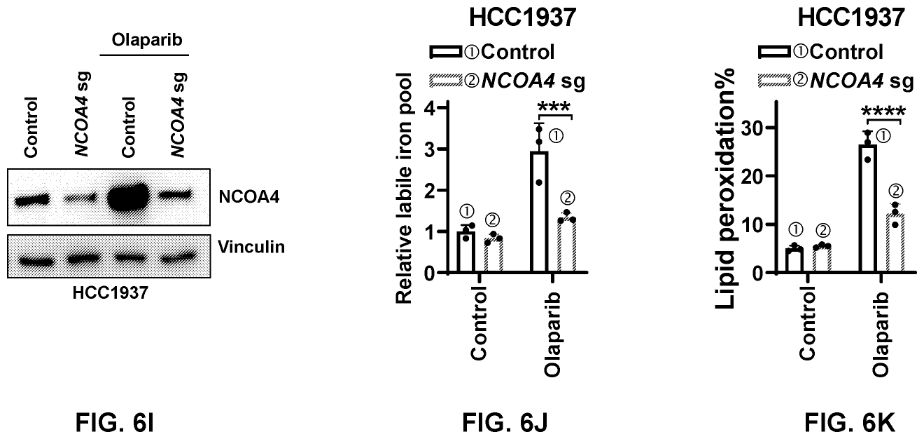

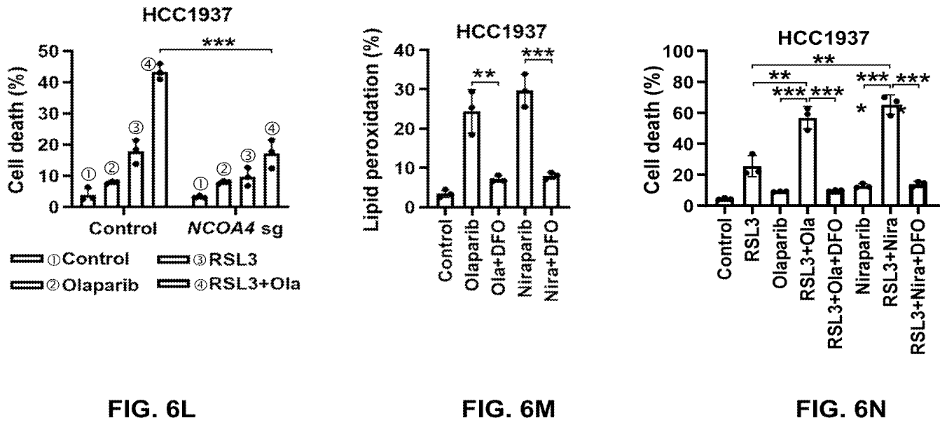

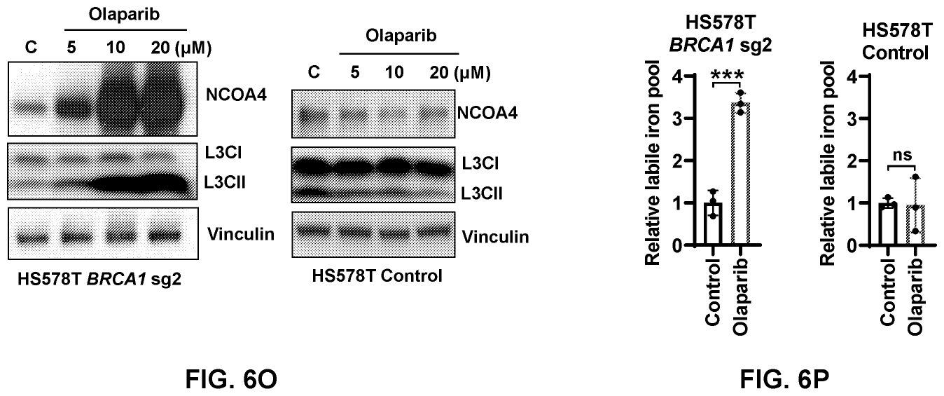

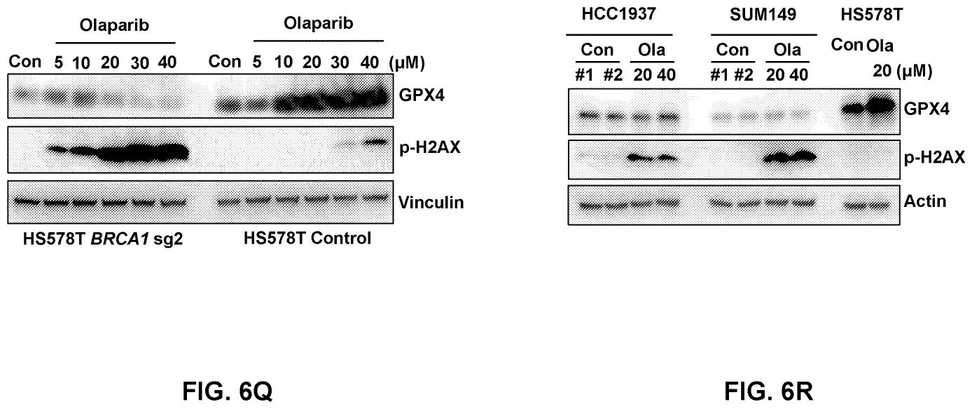

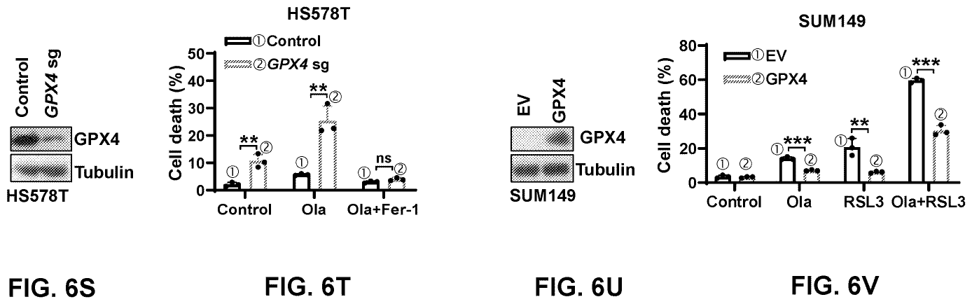

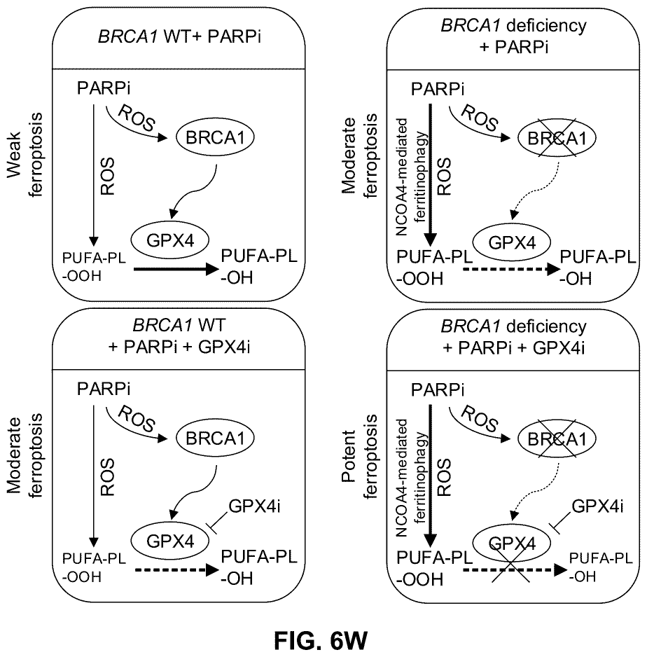

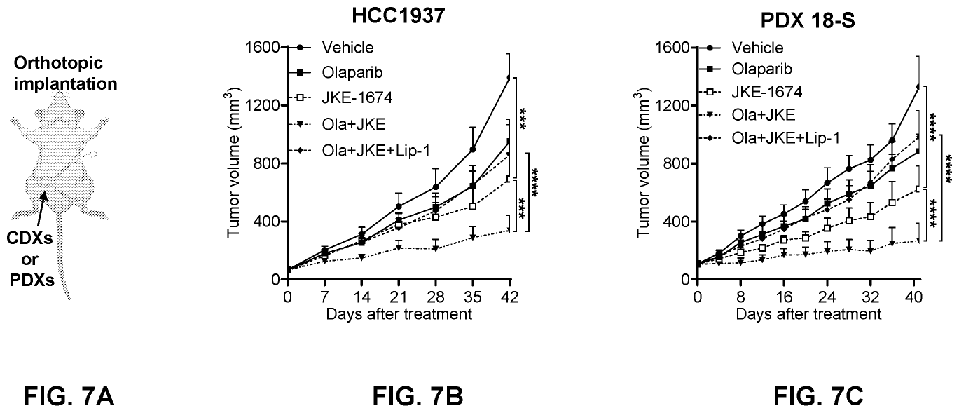

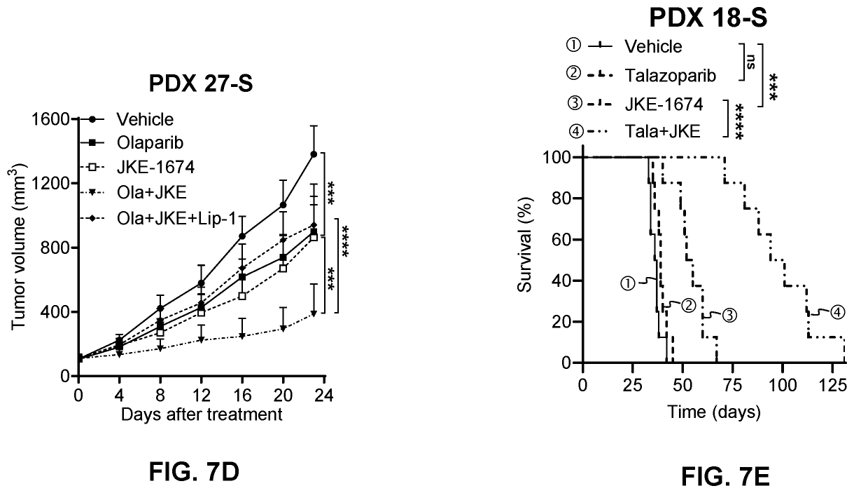

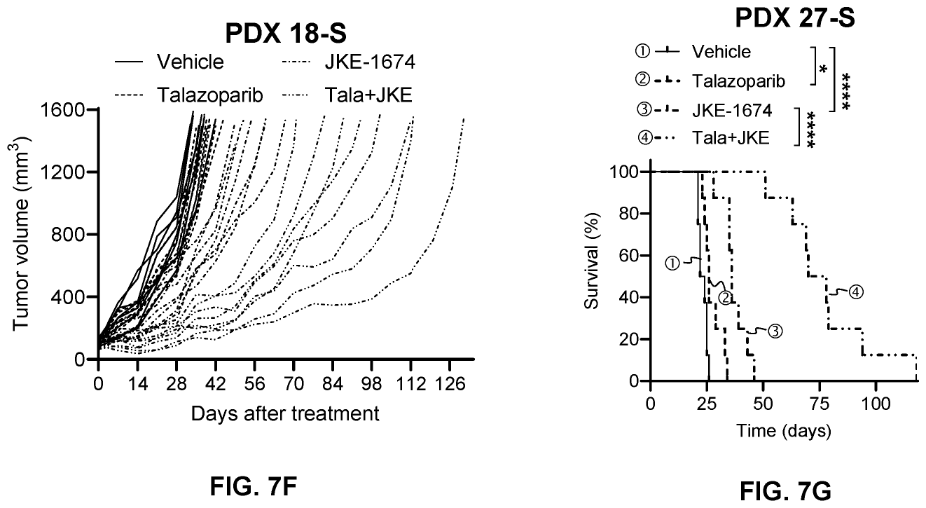

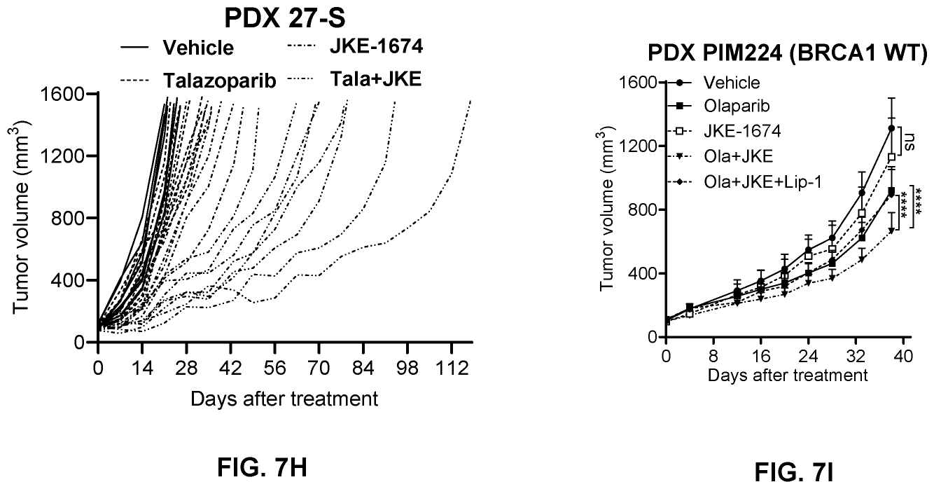

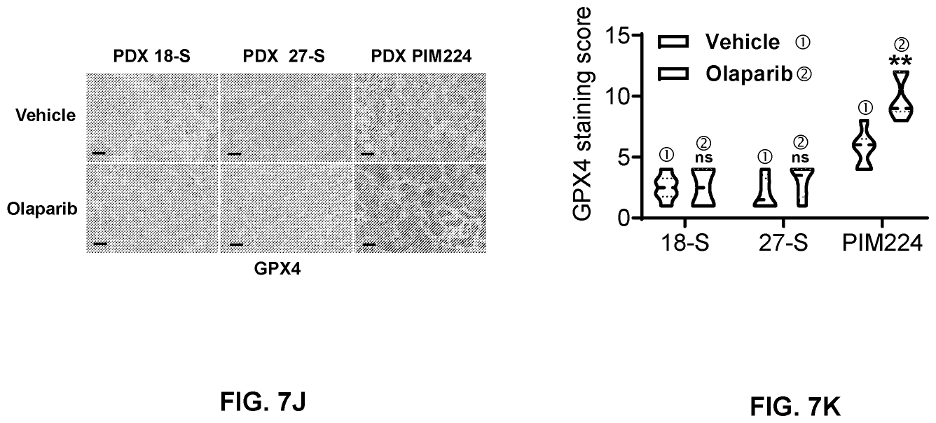

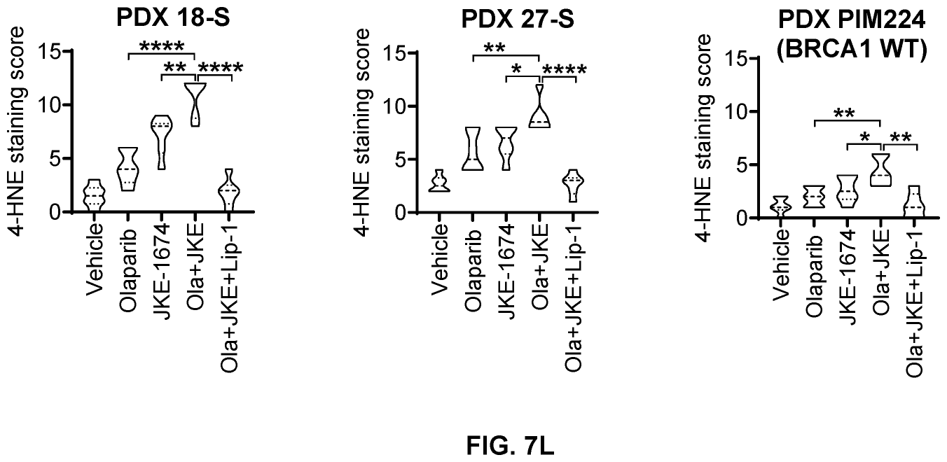

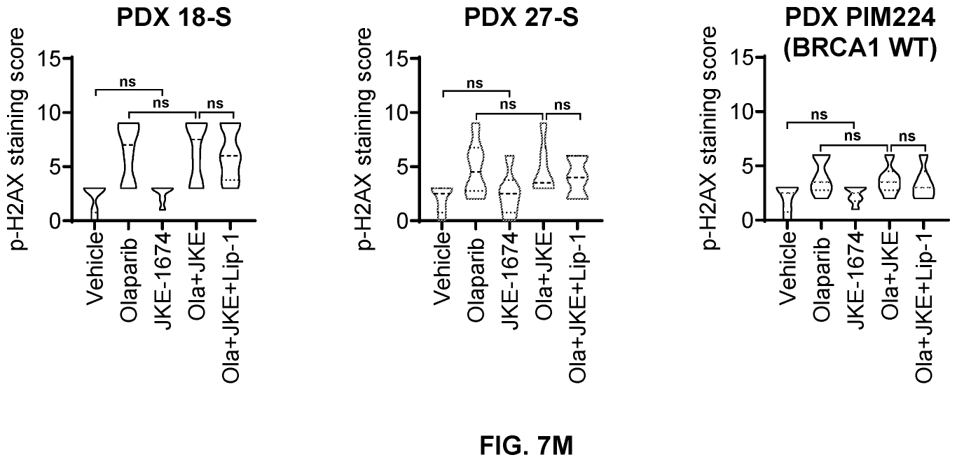

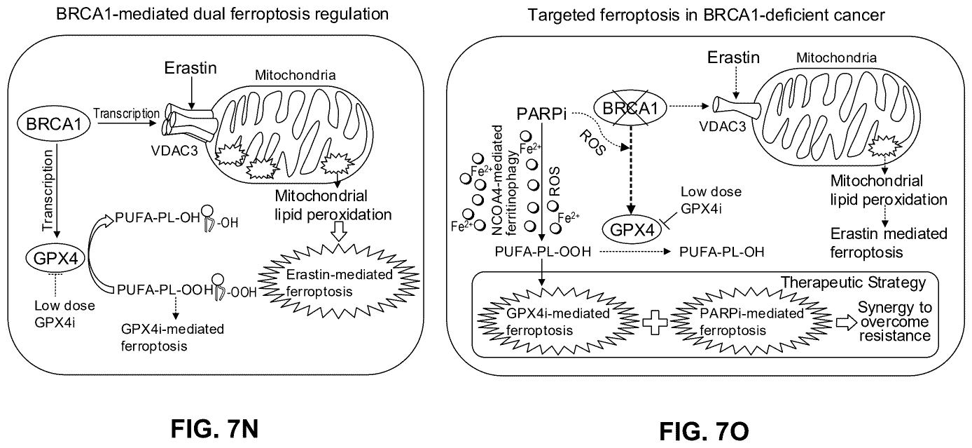

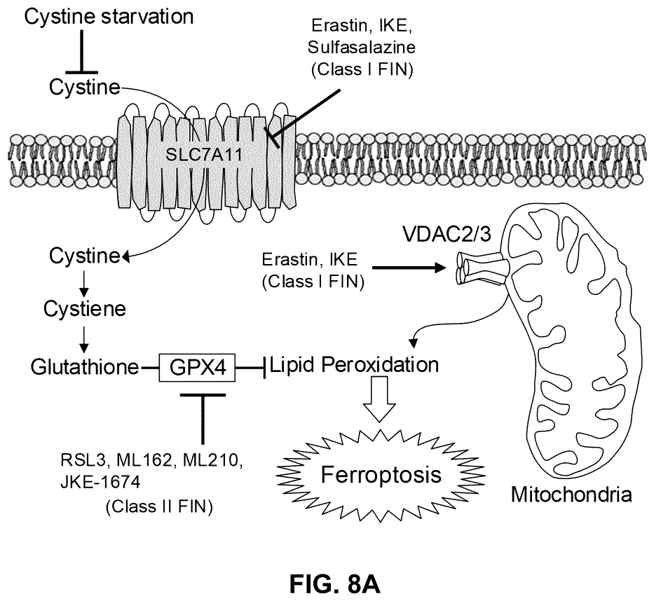

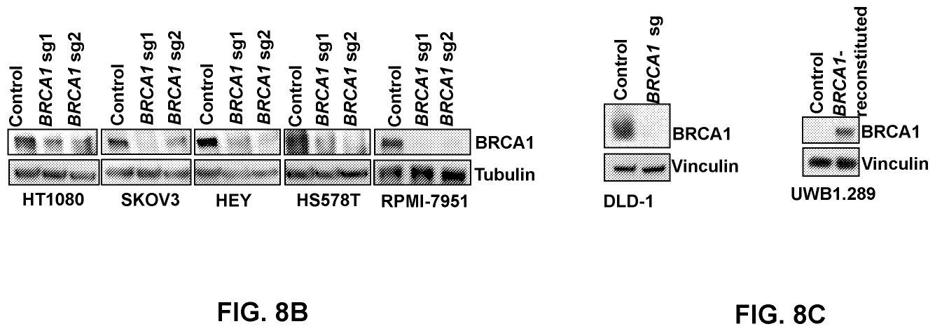

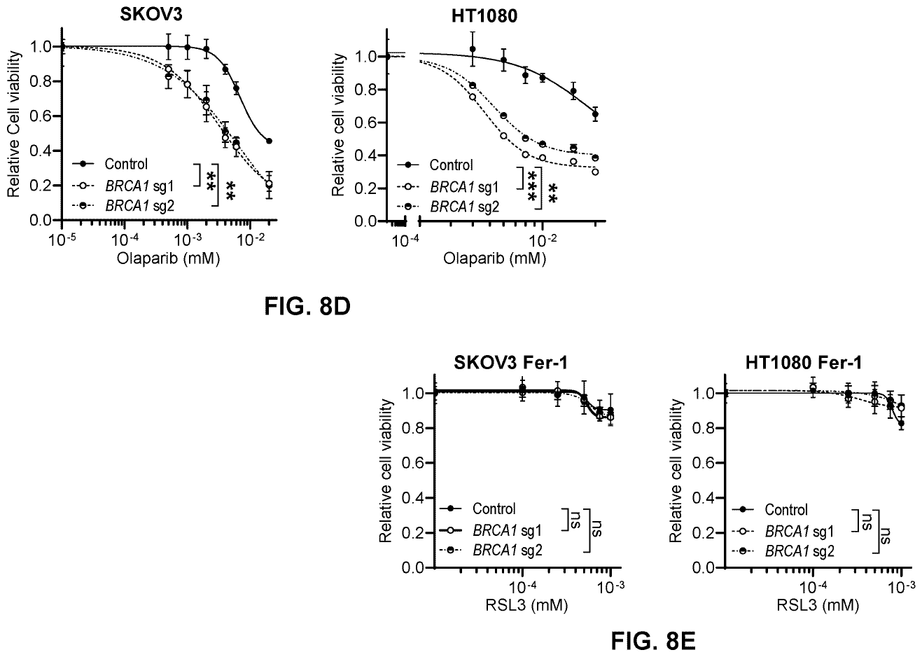

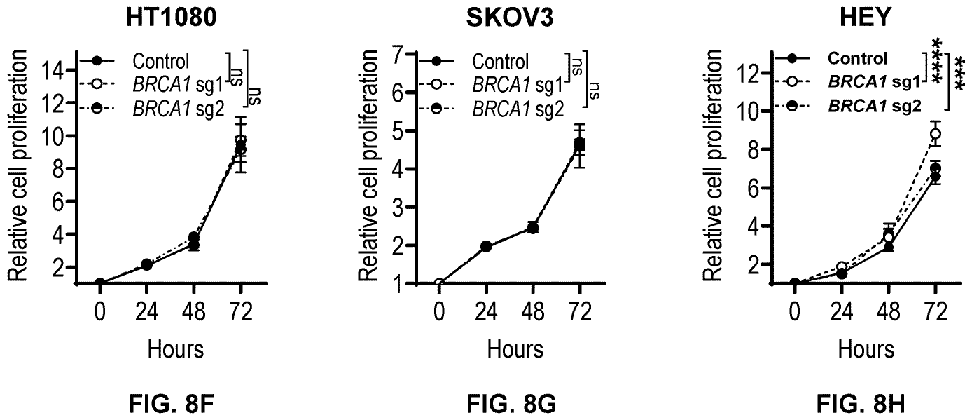

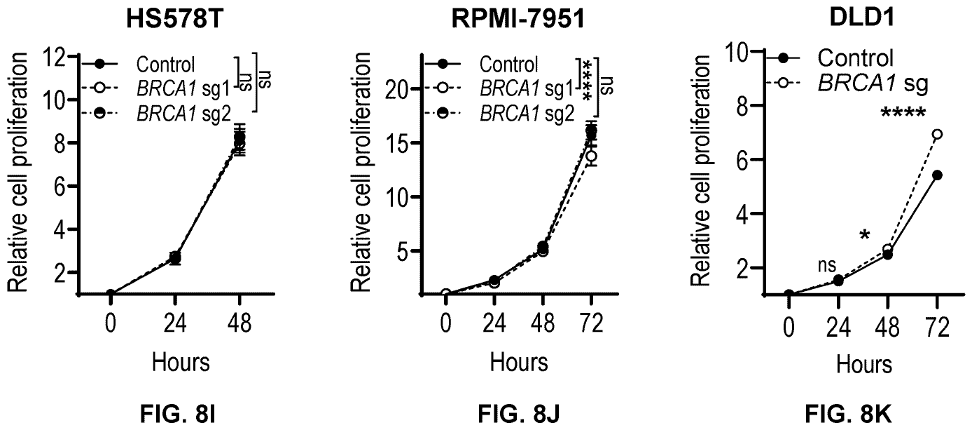

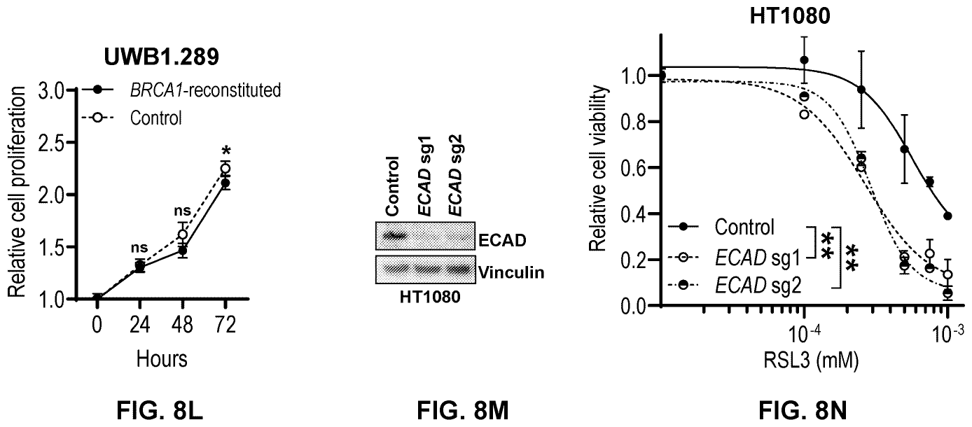

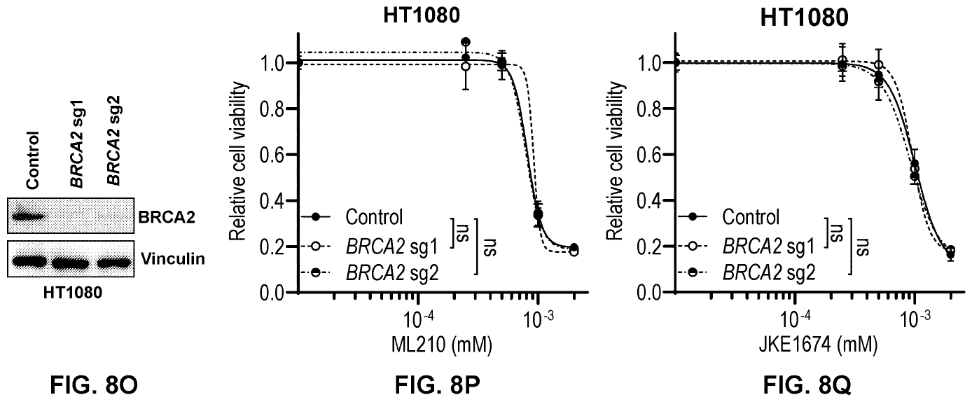

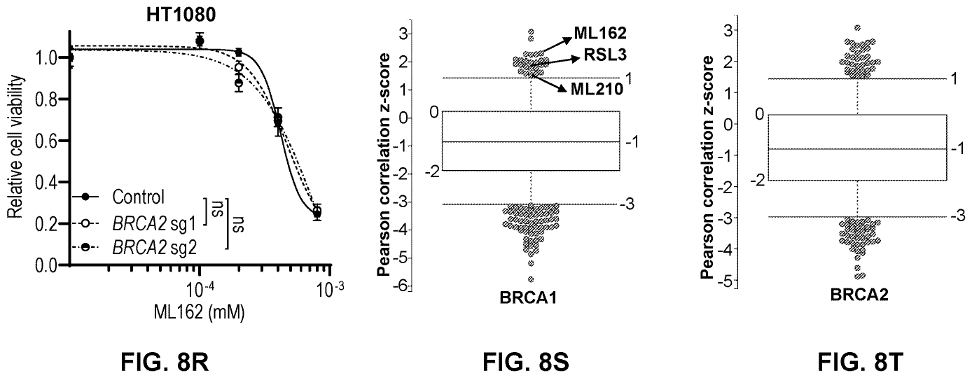

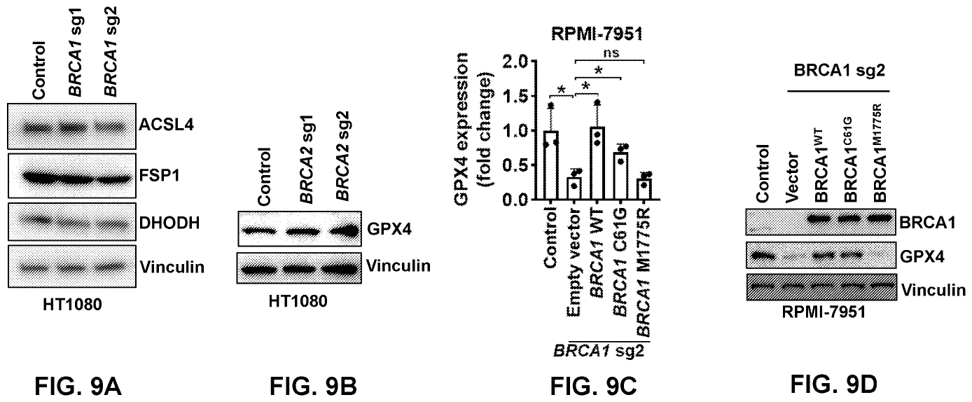

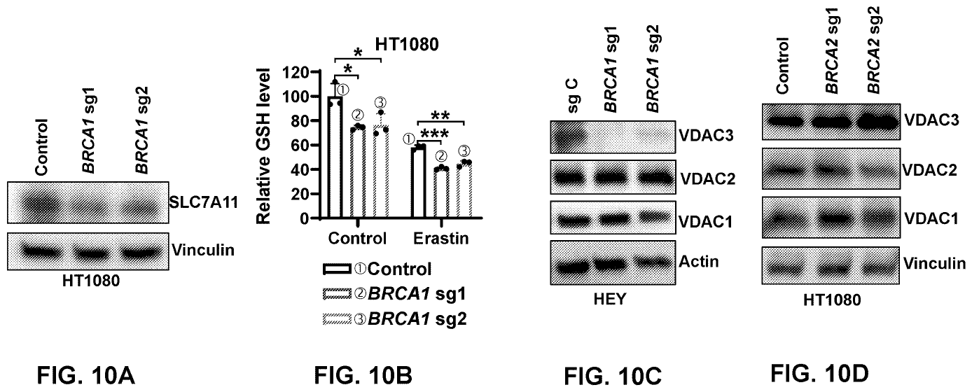

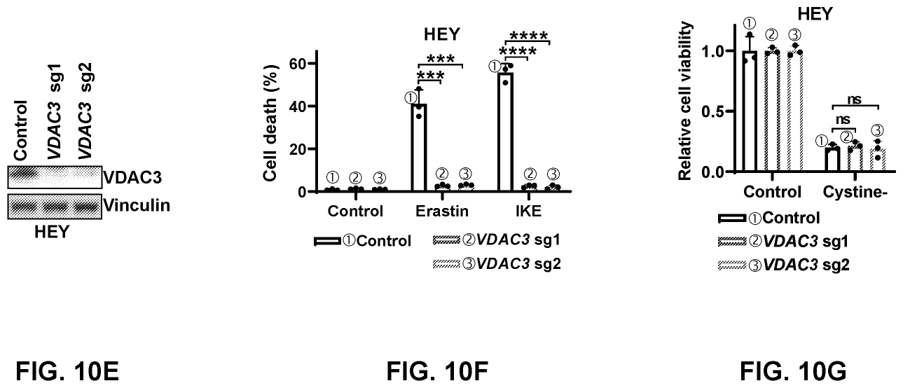

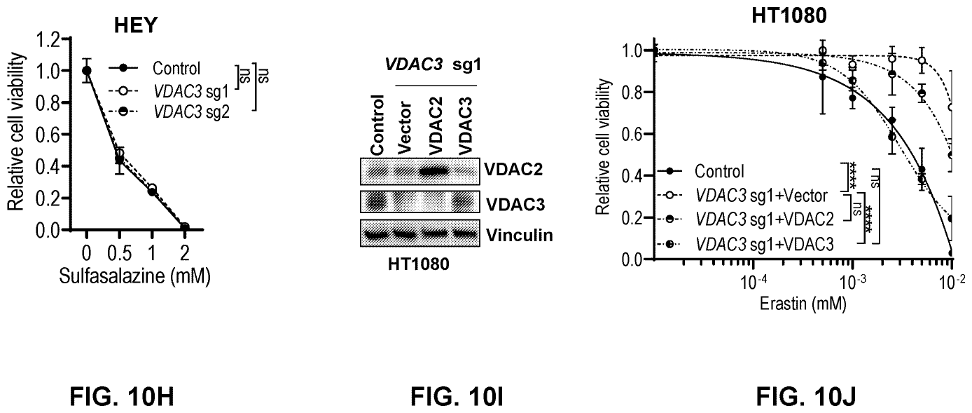

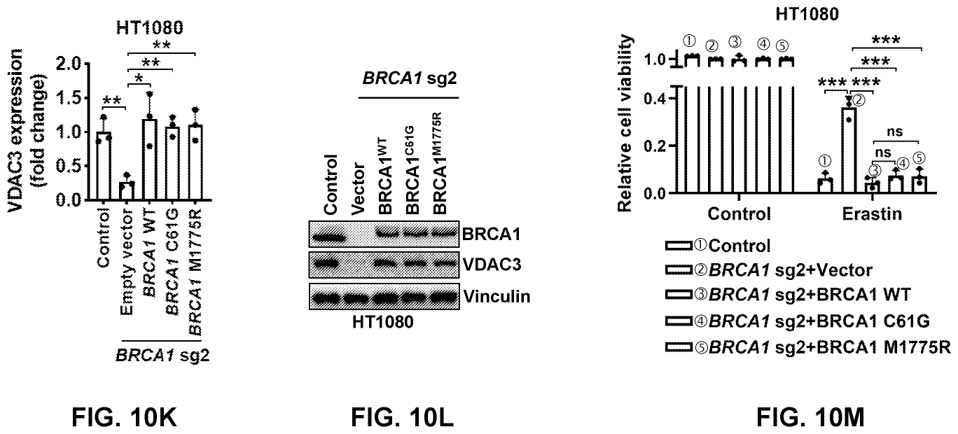

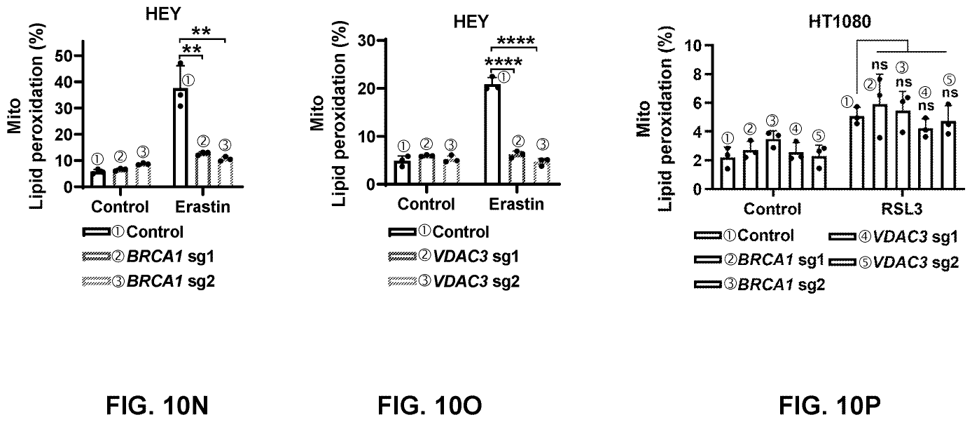

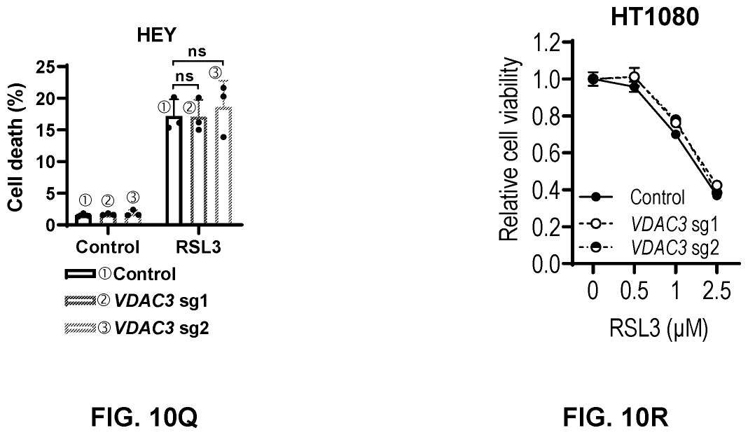

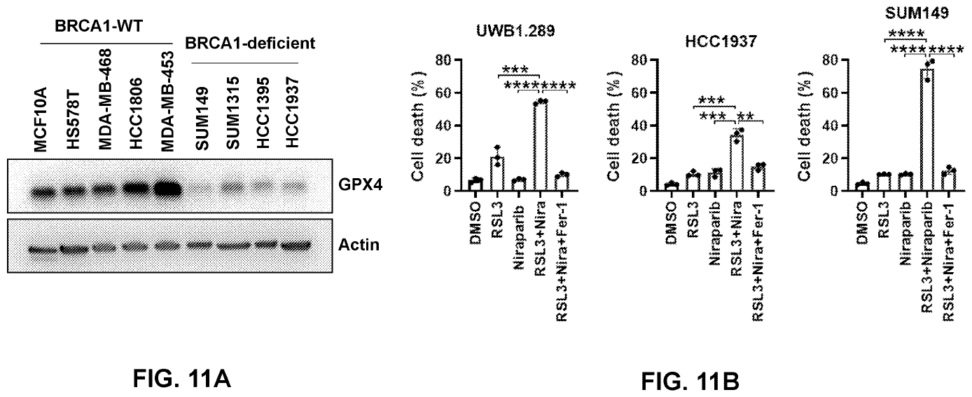

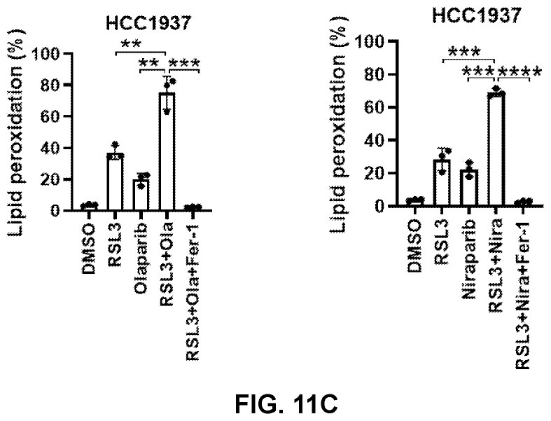

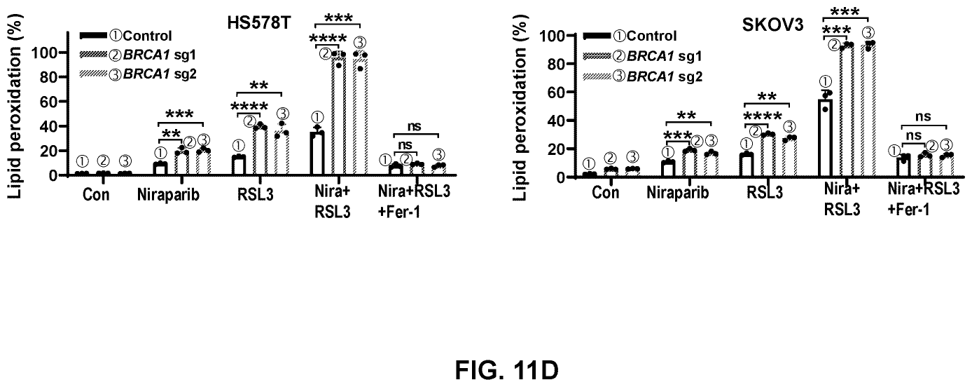

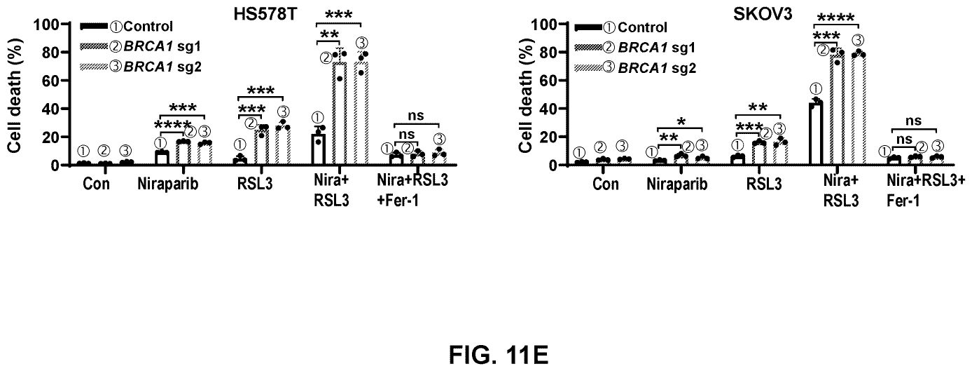

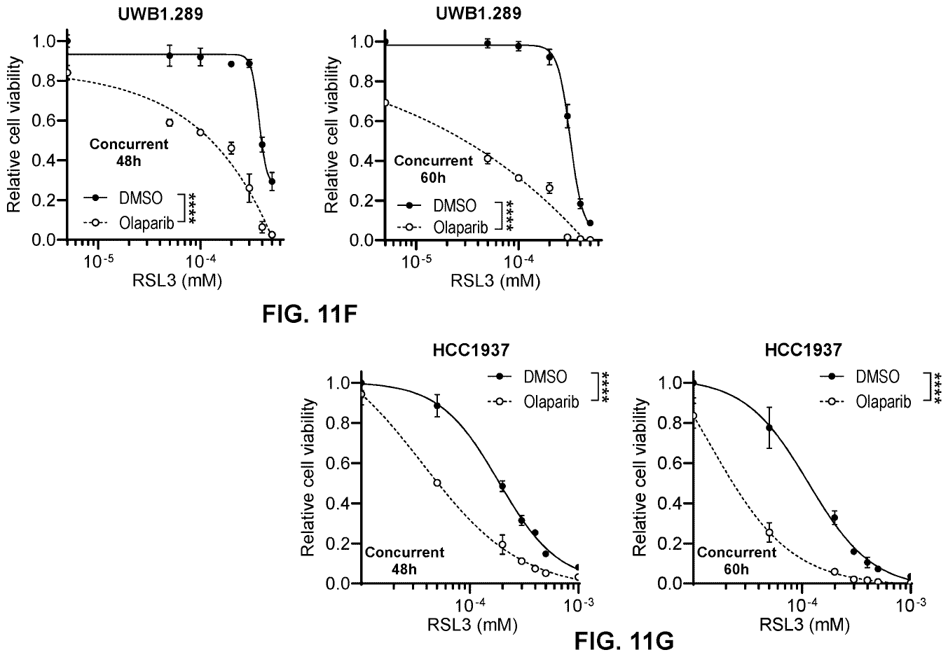

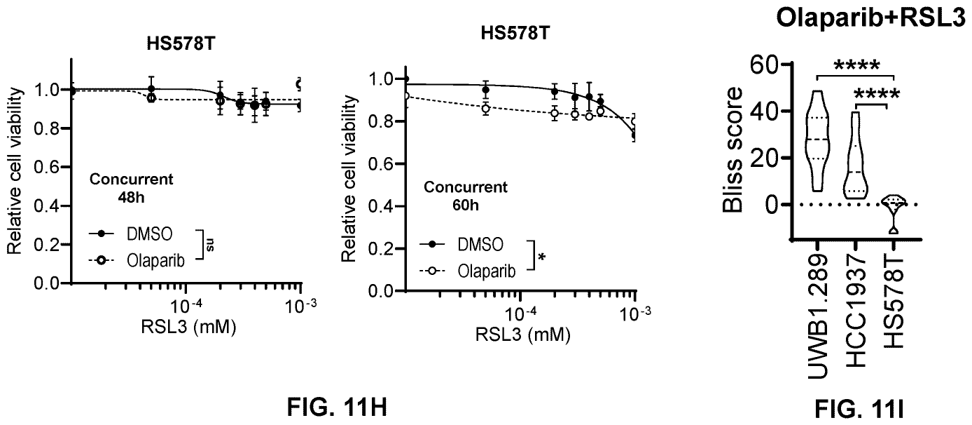

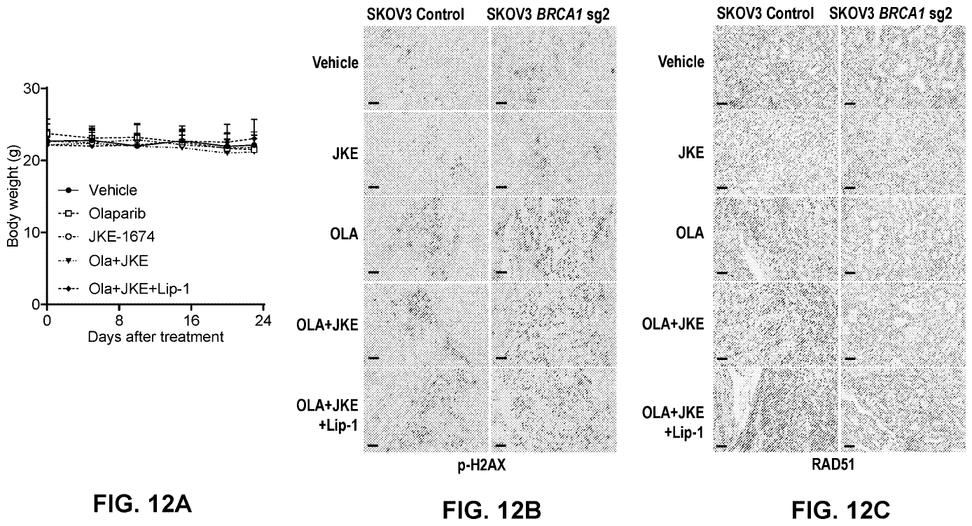

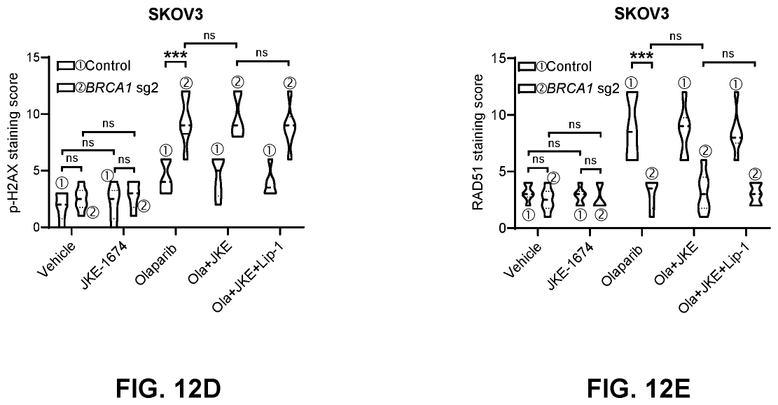

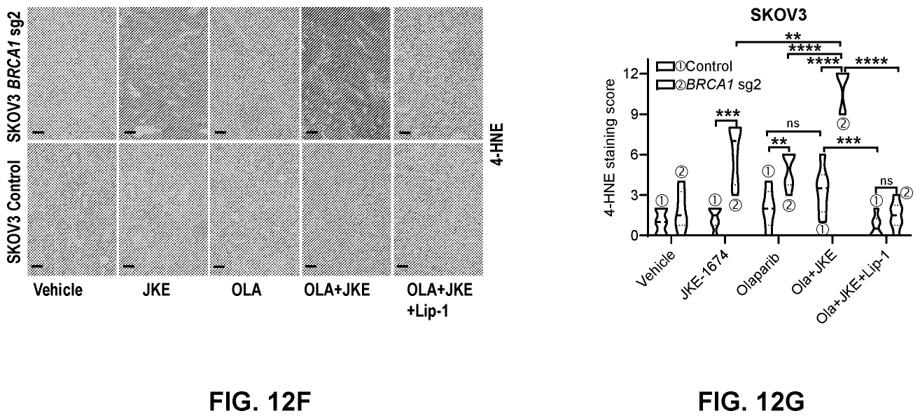

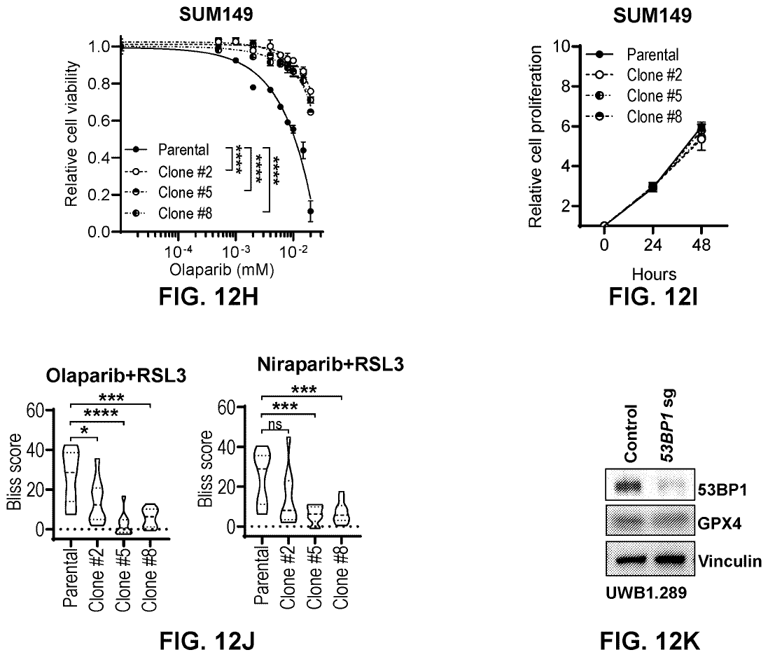

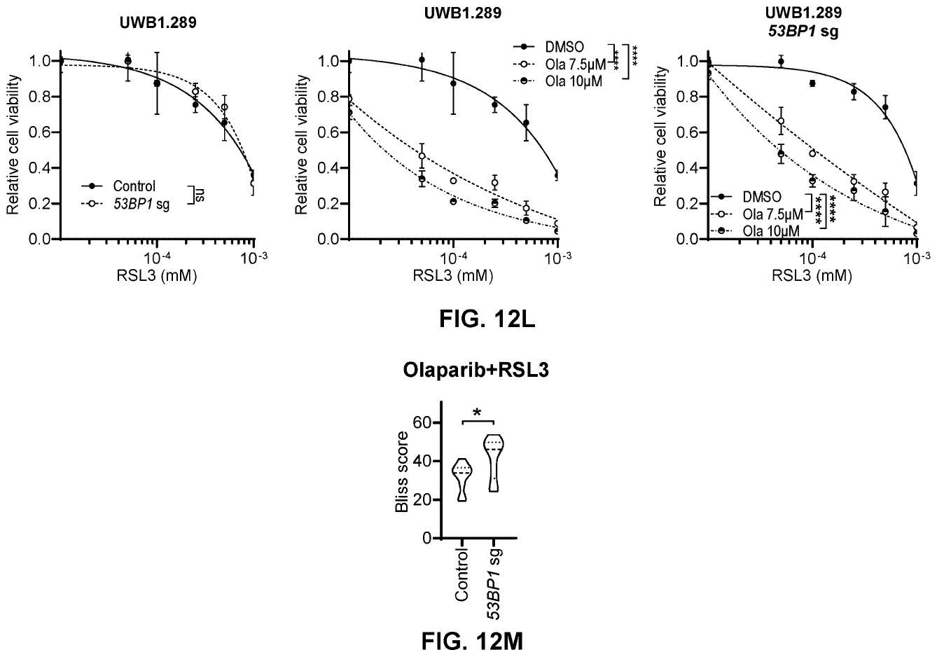

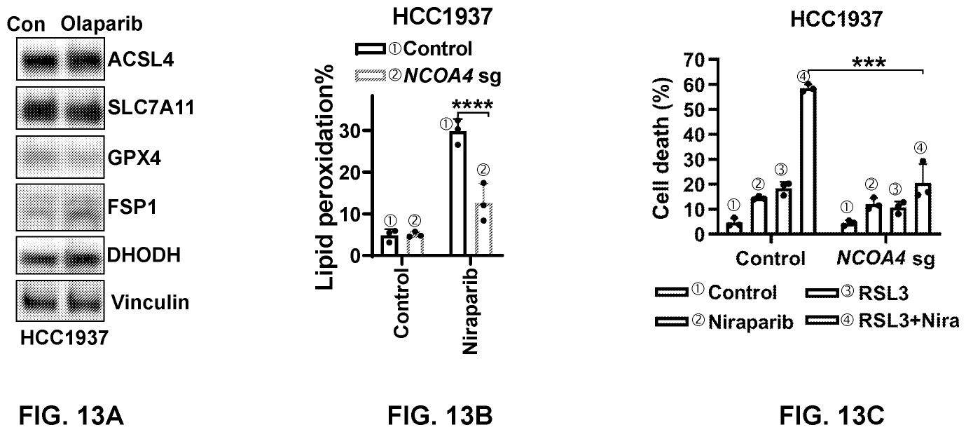

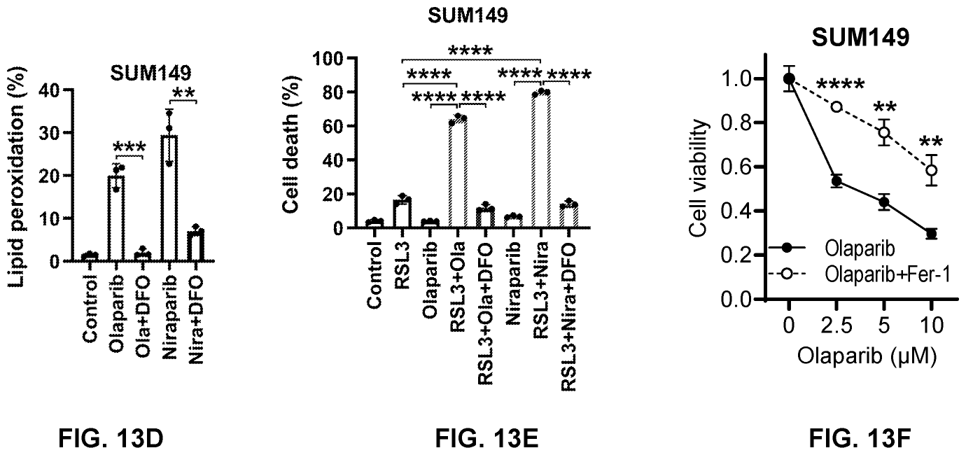

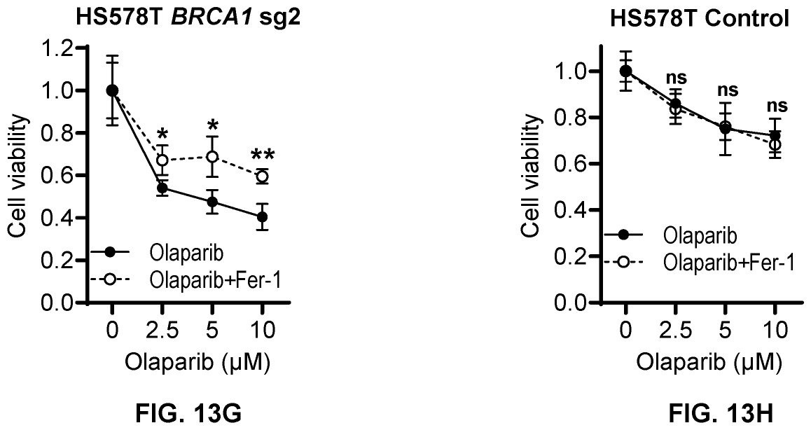

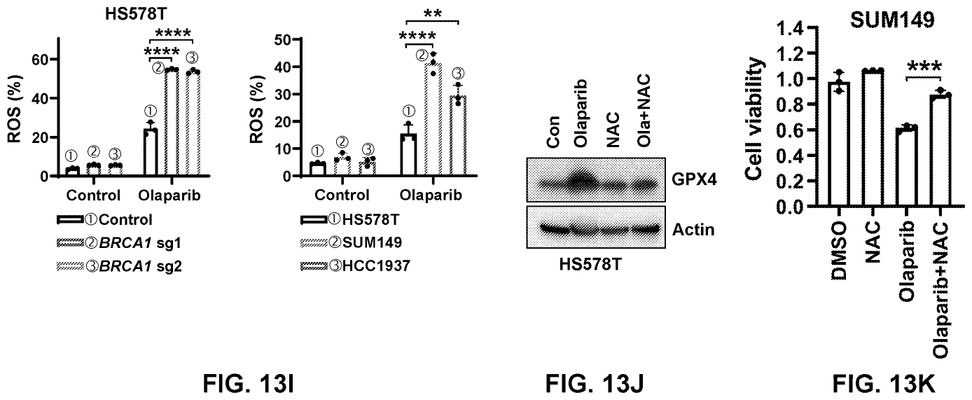

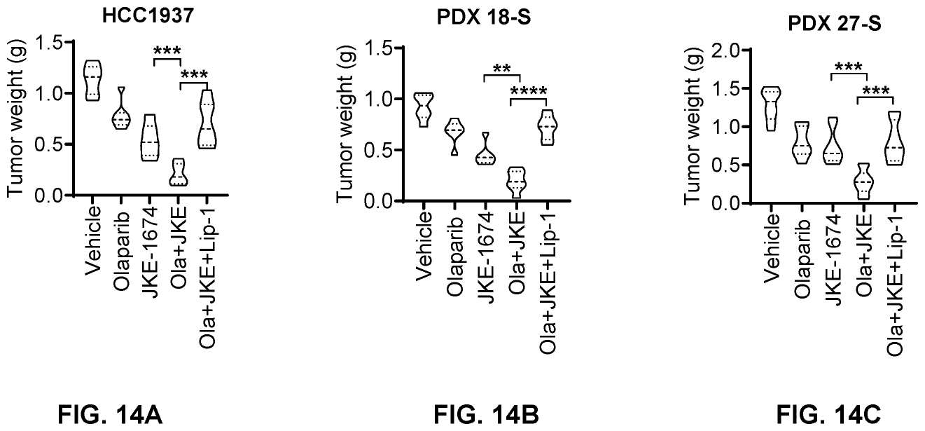

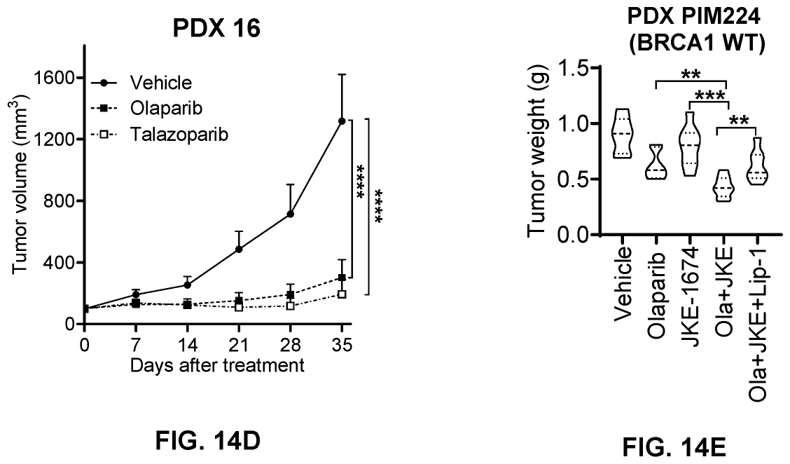

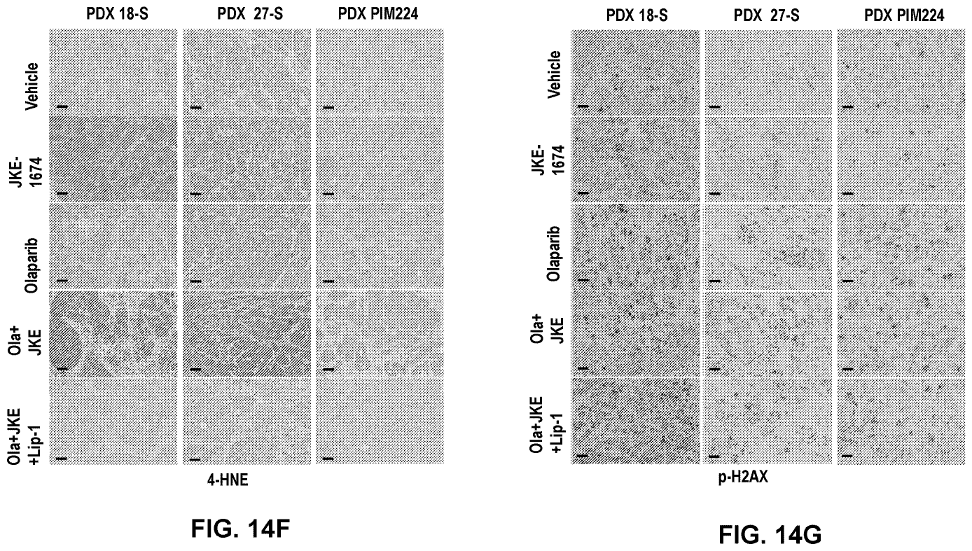

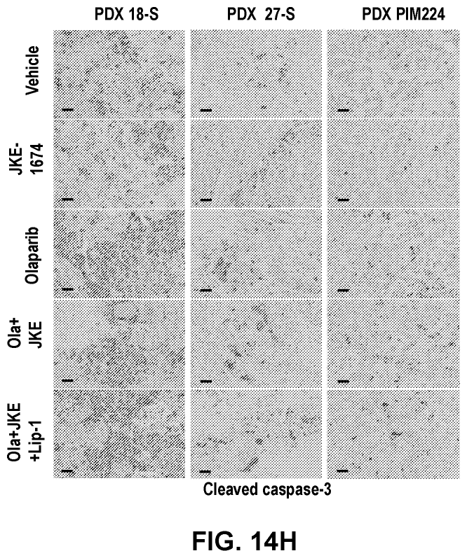

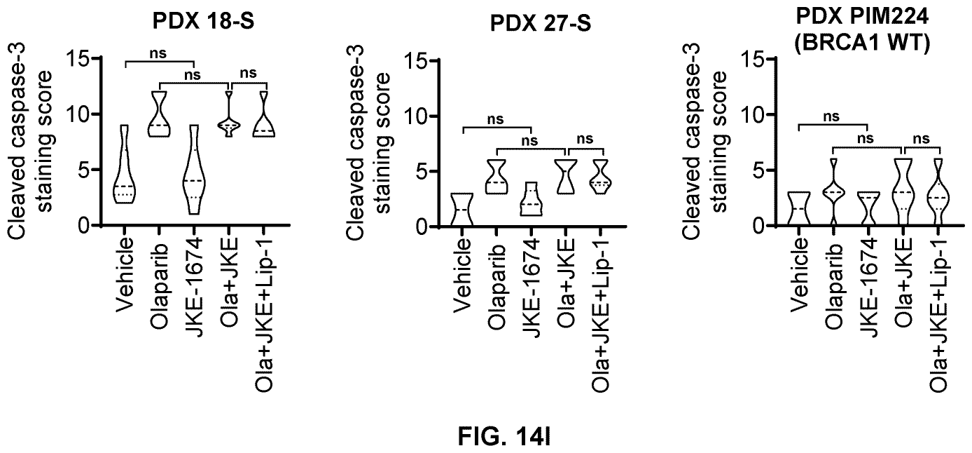

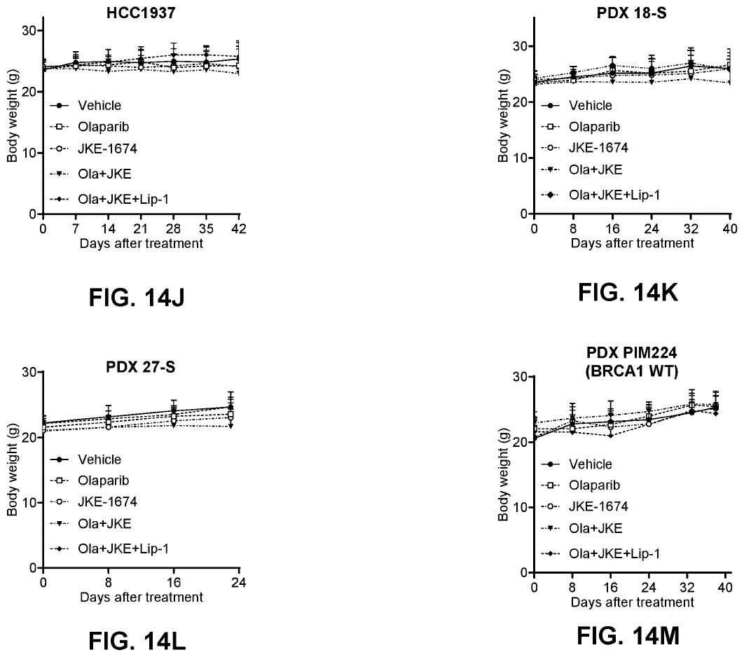

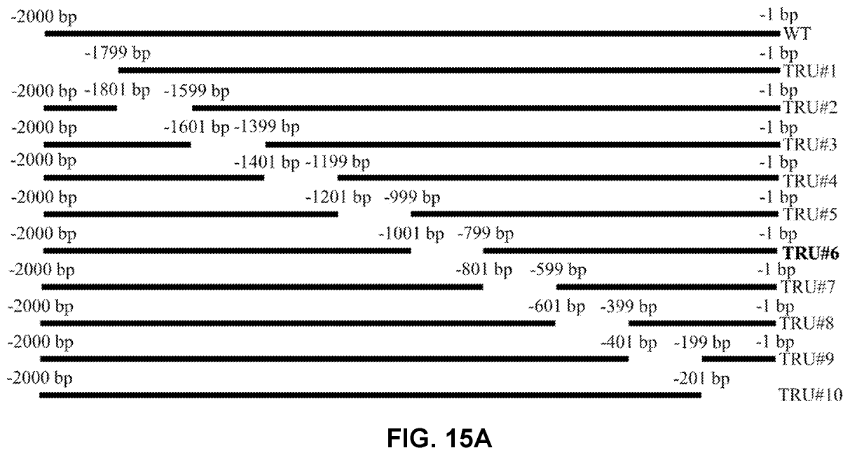

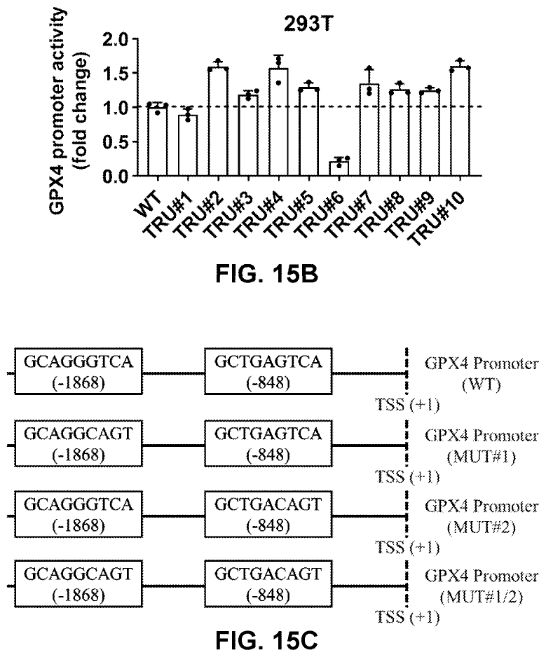

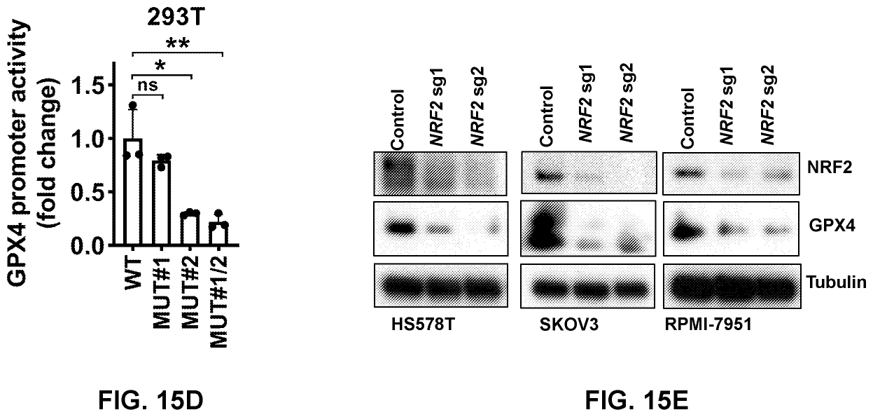

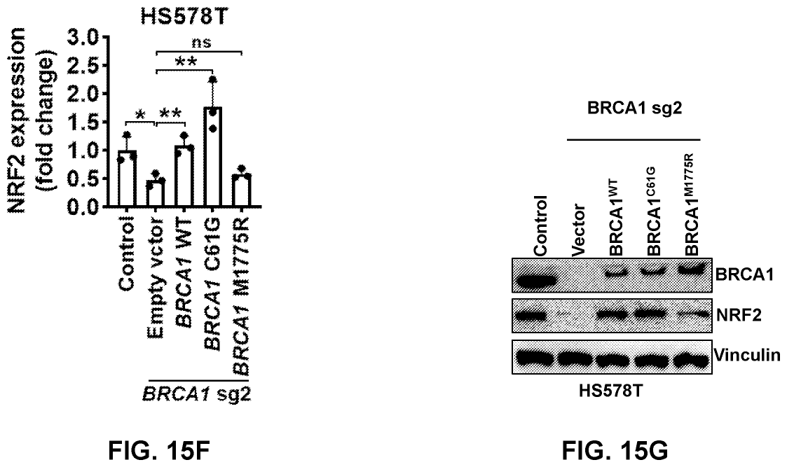

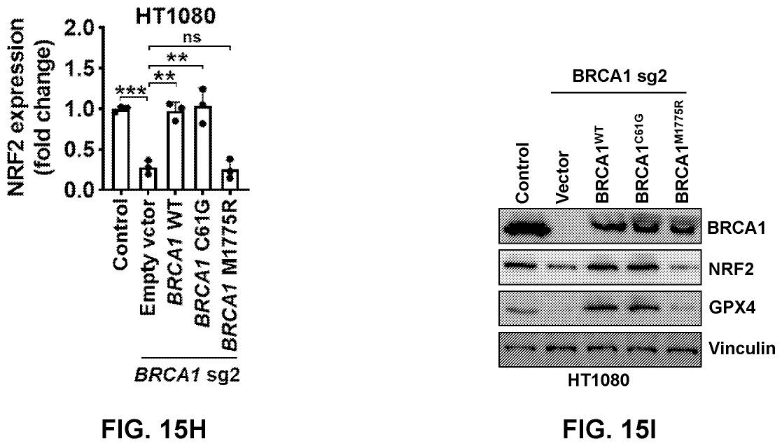

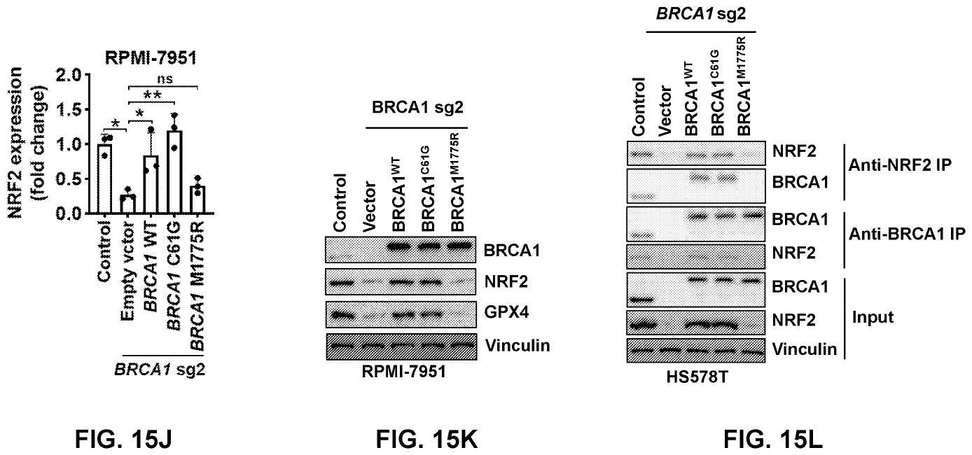

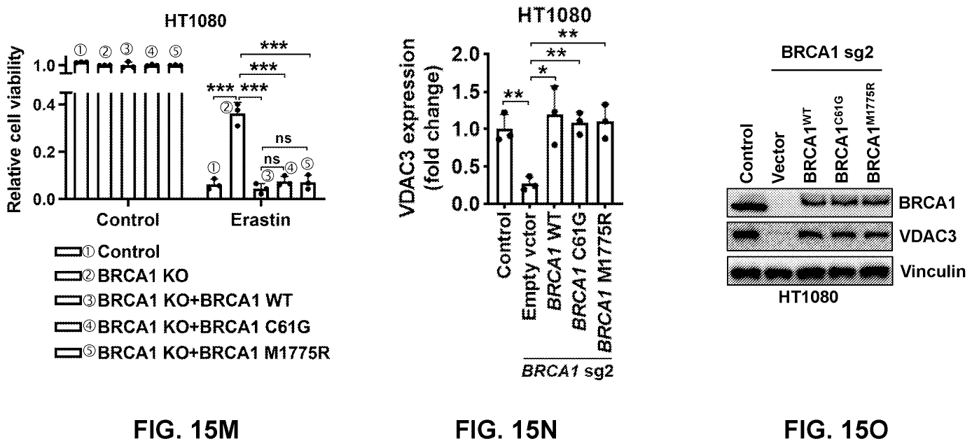

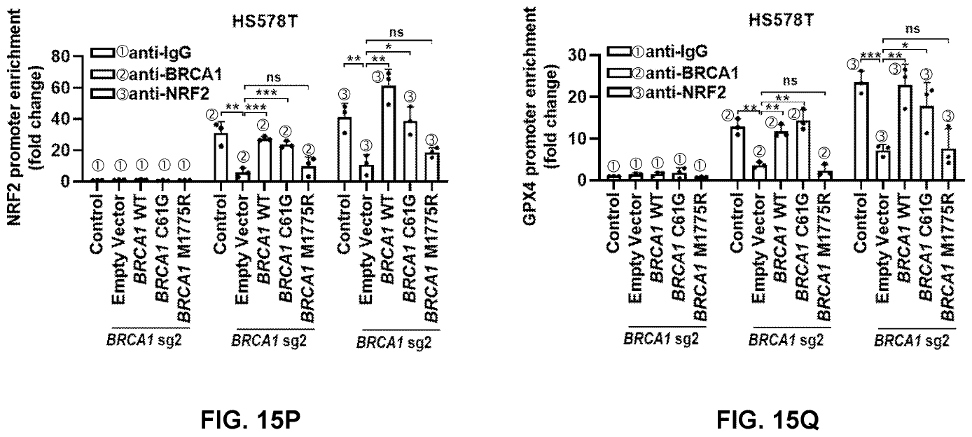

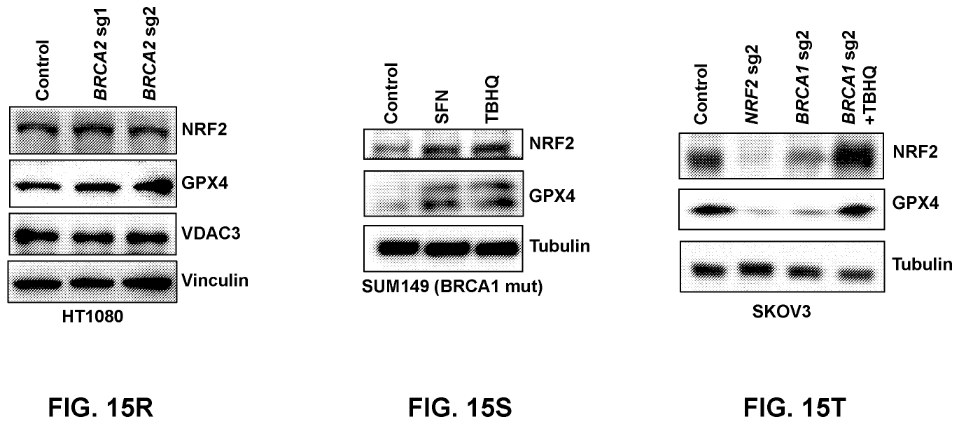

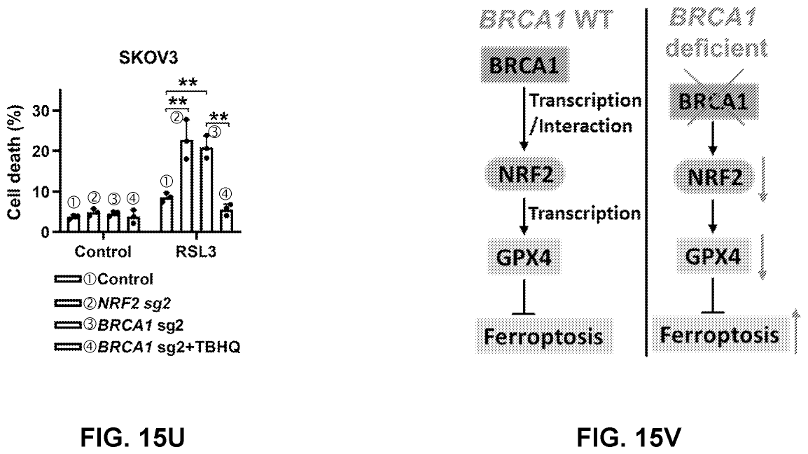

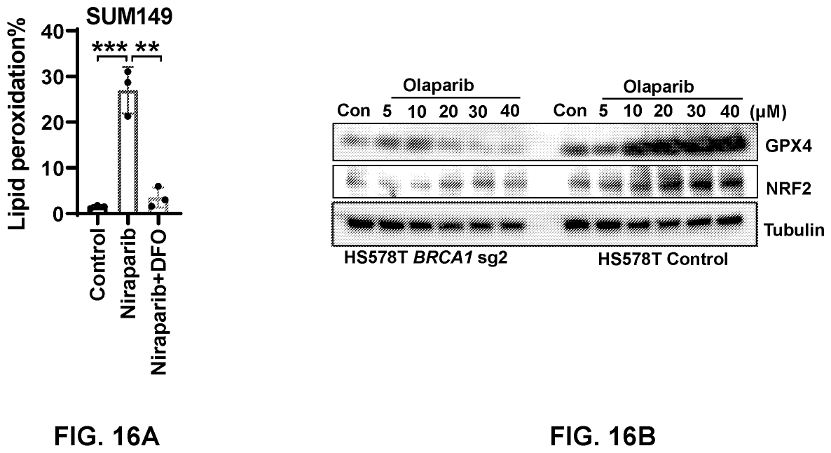

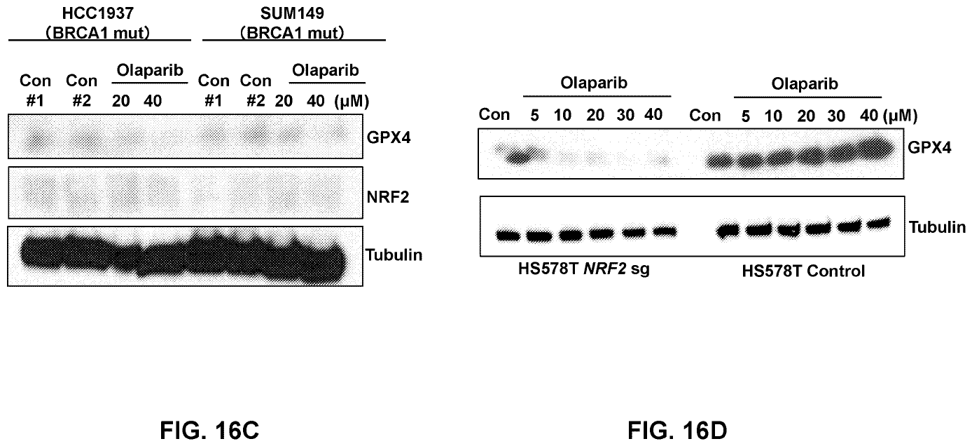

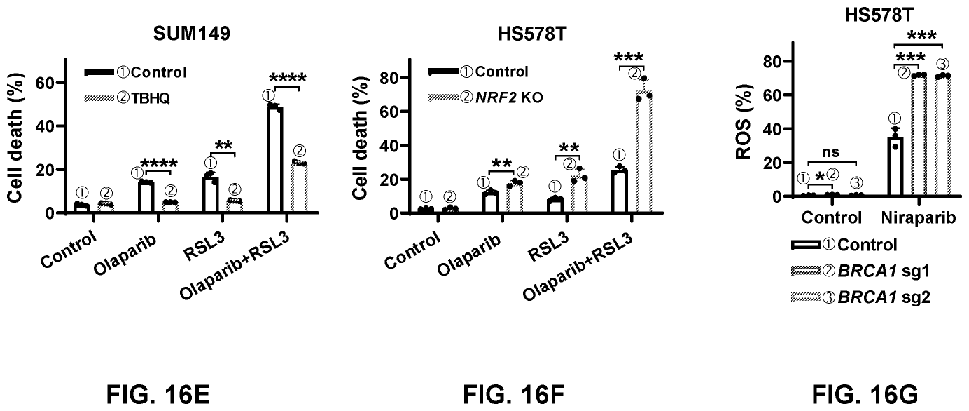

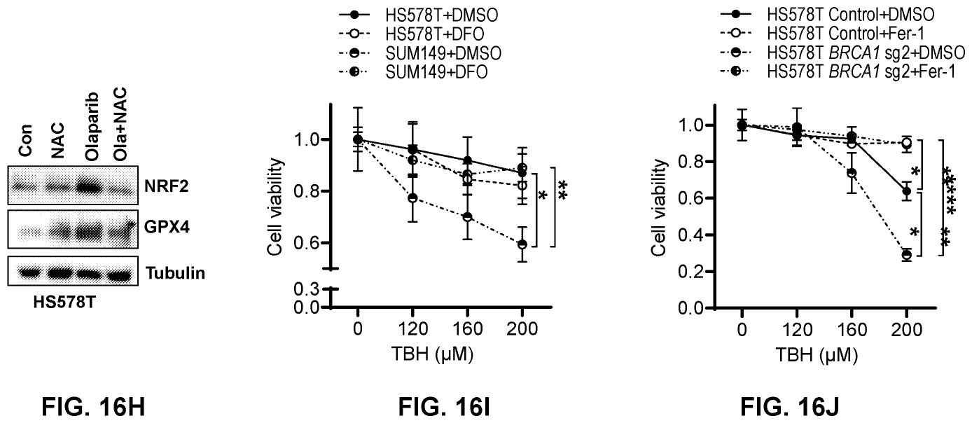

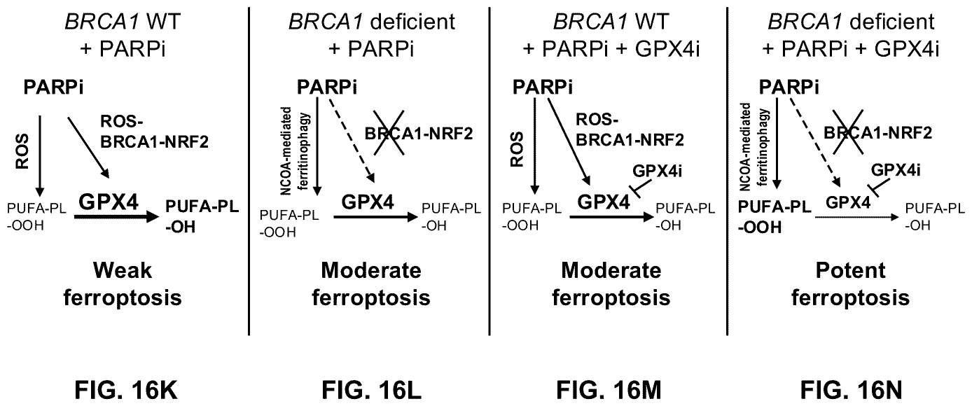

Ferroptosis phenotypes induced by class 1 FINs (SLC7A11 inhibitors) in BRCA1-deficient cells was examined. BRCA1 deficiency dramatically attenuated Erastin-induced lipid peroxidation and ferroptosis (FIG. 3A-3D). Re-expression of WT BRCA1 in BRCA1 sgRNA infected cells restored Erastin-induced ferroptosis (FIG. 3E). Similarly, BRCA1 deficiency remarkably repressed ferroptosis induced by imidazole ketone erastin (IKE, an Erastin analogue; FIG. 3F). Besides Erastin and IKE, sulfasalazine and cystine starvation are additional class I FINs that induce ferroptosis by inhibiting SLC7A11-mediated cystine uptake or limiting extracellular cystine availability (FIG. 8A). BRCA1 deficiency did not affect cells' sensitivity to sulfasalazine or cystine starvation (FIGS. 3G and 3H). In contrast, BRCA2 deficiency did not affect ferroptosis induced by class I FINs (FIG. 3I-3L).

Erastin has a dual effect on ferroptosis: it not only blocks SLC7A11-mediated cystine import and depletes intracellular glutathione pools, but also has a gain-of-function effect on mitochondrial voltage dependent anion channel (VDAC) 2 and 3 (FIG. 8A); consequently, SLC7A11 knockdown promotes whereas VIA (2 3 deficiency blocks Erastin-induced ferroptosis [18-19]. The results show that BRCA1-deficient cells exhibited moderately decreased SLC7A11 and intracellular glutathione levels (FIGS. 10A and 10B), which cannot explain Erastin resistance phenotypes observed in these cells. On the other hand, BRCA1 deficiency decreased the levels of VDAC3, but not VDAC1 or VDAC2 (FIGS. 4A, 4B, and FIG. 10C). As a comparison, BRCA2 deficiency did not affect expression levels of different VDAC members (FIG. 10D), which is consistent with our observations that BRCA2 deletion did not affect cellular sensitivity to Erastin or IKE (FIGS. 3I and 3J). Importantly, VDAC3 deletion largely abolished Erastin- or IKE-induced lipid peroxidation and ferroptosis (FIGS. 4C-4F, 10E, and 10F), but did not affect cellular sensitivity to cystine starvation or sulfasalazine (FIGS. 4G, 4H, 10G, and 10H), thereby phenocopying BRCA1 deficiency in these cancer cells. Overexpression of VDAC2 had a marginal effect on Erastin-induced ferroptosis in VDAC3-KO cells; as a control, VDAC3 expression re-sensitized VDAC3-KO cells to Erastin-induced ferroptosis to the level similar to that in WT (control) cells (FIGS. 10I and 10J). Furthermore, BRCA1 and VDAC3 double deficiency did not further enhance Erastin resistance compared to either single gene deficiency (FIGS. 4I and 4J), suggesting that BRCA1 and VDAC3 operate in the same signaling axis to regulate Erastin-induced ferroptosis.

To further examine how BRCA1 regulates VDAC3 expression, analyses of BRCA1 ChIP-seq datasets from GEO revealed a strong binding of BRCA1 on the VDA (3 promoter in diverse cancer cell lines (FIG. 4K). ChIP analyses confirmed these findings (FIG. 4L), suggesting that IT) A (′3 is a transcriptional target of BRCA1. Re-expression of either BRCA1-C61G or BRCA1-M1775R mutant, similar to BRCA1-WT re-expression, restored VDAC3 expression (FIGS. 10K and 10L) and Erastin-induced ferroptosis in BRCA1-deficient cells (FIG. 10M), suggesting that BRCA1 regulates GPX4 or VDAC3 expression and RSL3-or Erastin-induced ferroptosis through different mechanisms.

How VDAC3 is required for Erastin-induced ferroptosis remains unclear but might relate to its function to transport metabolites and ions across mitochondrial membrane to support mitochondrial metabolism [20]. In line with this, mitochondrial metabolism has been shown to drive Erastin-induced ferroptosis [5, 6]. BRCA1 or VDAC3 deficiency largely abolished mitochondrial lipid peroxidation induced by Erastin (FIGS. 4M, 4N, 10N, and 10O). In addition, whereas treatment with the radical-trapping antioxidant TEMPO or ferrostatin-1 completely suppressed Erastin-induced ferroptosis in wild-type (WT), BRCA1- or VDAC3-deficient cells, MitoTEMPO [7] treatment largely restored cell viability in Erastin-treated WT cells but did not provide a protective effect in Erastin-treated BRCA1- or VDAC3-deficient counterparts (FIG. 4O). RSL3 induced weak mitochondrial lipid peroxidation, and BRCA1 or VDA (3 deficiency did not affect mitochondrial lipid peroxidation under RSL3 treatment (FIG. 10P). Together, these data suggest that BRCA1 controls VDAC3 expression and that the BRCA1-VDAC3 axis promotes Erastin-induced ferroptosis mainly through inducing mitochondrial lipid peroxidation. It should be noted that VIA (3 deletion did not affect RSL3-induced ferroptosis (FIGS. 10Q and 10R); therefore, BRCA1 regulates GPX4 inhibitor-induced ferroptosis through VDAC3-independent mechanisms.

### Example 5: PARP Inhibitor Synergizes with GPX4 Inhibitor to Induce Ferroptosis in BRCA1-Deficient Cancer Cells and Xenografts

Since BRCA1 deficiency sensitizes cancer cells to both PARP inhibitor and GPX4 inhibitor, the combinatorial effects of PARP inhibitor and GPX4 inhibitor in BRCA1-WT and -deficient cancer cells were also studied. It was examined that PARPi (niraparib or olaparib) in combination with GPX4i (RSL3) demonstrated higher synergism in BRCA1-mutant UWB1.289 cells (or BRCA1-WT HS578T cells infected with BRCA1 sgRNAs) than in their BRCA1-reconsituted counterparts (or their WT counterparts) (FIG. 5A-E). In a collection of triple negative breast cancer cell lines, BRCA1-mutant cells (SUM149, HCC1937, HCC1395, and SUM1315) exhibited lower GPX4 expression and more synergism to the PARPi and GPX4i combination therapy than did BRCA1-WT cells (HS578T, MDA-MB-468, HCC1806, MDA-MB-453) or MCF10A cells (an immortalized mammary epithelial cell line) (FIGS. 5D, 5F-5H, and 11A).

PARP inhibitor in combination with GPX4 inhibitor induced potent lipid peroxidation and cell death in BRCA1-mutant SUM149 cells; notably, cell death induced by PARP inhibitor+GPX4 inhibitor combination could be largely abolished by the ferroptosis inhibitor ferrostatin-1 or liproxstatin-1, but not by the apoptosis inhibitor Z-VAD or the necroptosis inhibitor Nec-1s (FIGS. 5I, 5J, 11B, and 11C). Likewise, the combination treatment induced much more potent lipid peroxidation and ferroptosis in BRCA1-depleted HS578T or SKOV3 cells than in their WT counterparts (FIGS. 11D and 11E). In the previous data, RSL3 was used as the GPX4 inhibitor, these observations were further confirmed using the GPX4 inhibitor JKE-1674 (FIGS. 5K and 5L).

It is known that typical concentrations used in the literature of GPX4i (e.g., RSL3) exerts a significantly more potent cell-killing effect than PARPi (e.g., Olaparib) when treated for the same durations. Given these distinct kinetics, in our co-treatment experiments previously described, the treatment duration was adjusted by initially exposing cells to PARPi for 24 hours, followed by a subsequent co-treatment with both PARPi and GPX4i for an additional 12-18 hours. Investigations were further extended by co-treating cells with PARPi and lower doses of GPX4i for an extended treatment duration, without the pre-treatment of PARPi. It was confirmed that PARPi and GPX4i concurrent combination demonstrated more synergism in BRCA1-mutant cells than in BRCA1-WT cells (FIGS. 11F-11I).

Next, the efficacy of PARPi (olaparib)+GPX4i combination therapy in treating BRCA1-WT or -deficient SKOV3 cell-derived xenograft was examined. Because RSL3 is not suitable for in vivo treatment owing to their poor pharmacokinetics, in our preclinical studies, we used JKE-1674, a recently developed potent GPX4i with significantly improved pharmacokinetics in vivo [21]. It was shown that while olaparib and JKE-1674 single or combination treatment did not affect or only moderately suppressed BRCA1-WT xenograft tumor growth, olaparib or JKE-1674 single treatment had more dramatic tumor suppressive effect on BRCA1-deficient tumors than on BRCA1-WT counterparts, and the combination treatment blunted the growth of BRCA1-deficient tumors (FIGS. 5M and 5N). In addition, the ferroptosis inhibitor liproxstatin-1 significantly restored BRCA1-deficient xenograft tumor growth in the olaparib+JKE-1674 treatment group (FIGS. 5M and 5N), indicating that the combination therapy suppresses BRCA1-deficient tumor growth at least partly by inducing ferroptosis in tumors. These treatments did not cause any obvious toxicity or decrease the animals' weight (FIG. 12A).

Olaparib treatment resulted in increased staining for both p-H2AX and RAD51, indicative of elevated DNA damage and enhanced replication fork stability in olaparib-treated tumors; moreover, BRCA1 deficiency further augmented p-H2AX staining but decreased RAD51 staining under Olaparib treatment (FIGS. 12B-12E). Importantly, JKE-1674 or liproxstatin-1 treatment did not impact p-H2AX or RAD51 nuclear staining under vehicle or PARPi treatment conditions in either BRCA1-WT or -deficient tumors (FIGS. 12B-12E), suggesting that the anti-tumor effect of GPX4i is not related to its potential impact on DNA damage or replication fork stability. It was further shown that, under the single or combination treatment, BRCA1-deficient tumors exhibited increased staining for 4-HNE (a lipid peroxidation biomarker) compared to BRCA1-WT tumors, and liproxstatin-1 treatment suppressed 4-HNE staining (FIGS. 12F and 12G).

GPX4i was tested as either as a single treatment or in combination with PARPi, in two cellular models of PARPi-resistant BRCA1-mutant cancer cells. BRCA1 reversion represents an important mechanism driving PARPi resistance in BRCA1-deficient cancers [22-23]. Parental and several BRCA1-reverted SUM149 cell lines (clones #2, #5, and #8) were used as previously reported and confirmed that the restored BRCA1 expression and enhanced PARPi resistance in these BRCA1-reverted cell lines compared to their parental counterparts (FIGS. 5O and 12H).

BRCA1-reverted cells exhibited similar cell proliferation rates compared to the parental cells (FIG. 12I). We found that while #5 and #8 BRCA1-reverted cells displayed increased GPX4 expression, greater resistance to RSL3, and lack of response to the GPX4i+PARPi combination, #2 BRCA1-reverted cells maintained similar GPX4 expression and RSL3 sensitivity to the parental cells, and the GPX4i+PARPi combination exhibited effectiveness in this cell line (FIGS. 5O-5T and 12J). This suggests that GPX4 expression can be used as a potential biomarker for selecting patients with BRCA1 reversion mutations who may benefit from the combination therapy.

Further, it is known that 53BP1 deficiency restores HR defect and therefore promotes PARPi resistance in BRCA1-deficient cells [22-23]. We found that 53BP1 deletion in BRCA1-mutant UWB1.289 cells did not affect GPX4 levels or RSL3-induced ferroptosis (FIGS. 12K and 12L); importantly, although 53BP1 deletion conferred resistance to PARPi, PARPi continued to potentiate GPX4i-induced cell death in these cells, resulting in similar or even higher synergism scores in BRCA1-mutant cells with 53BP1-KO UWB1.289 cells than in 53BP1-WT counterparts (FIGS. 12L and 12M). These results underscore the potential of the combination of PARPi and GPX4i as an effective therapeutic strategy to counteract PARPi resistance engendered by BRCA1-independent HR restoration.

Together, the data suggest that PARP inhibitor synergizes with GPX4 inhibitor to induce ferroptosis in BRCA1-mutant/-deficient cancer cells and tumors.

### Example 6: NCOA4-Mediated Ferritinophagy Coupled with Defective GPX4 Induction Drives the Synergy Between PARP Inhibitor and GPX4 Inhibitor in BRCA1-Deficient Cancer Cells

Mechanistic interrogation of how PARP inhibitor synergizes with GPX4 inhibitor to induce ferroptosis in BRCA1-deficient cancer cells was investigated. First, experiments were done to examine whether PARP inhibitor treatment affected the expression levels of known ferroptosis regulators in BRCA1-deficient cancer cells, and found that olaparib or niraparib treatment significantly induced nuclear receptor coactivator 4 (NCOA4) expression in SUM149 and HCC1937 cells (FIGS. 6A-6F), but not that of other ferroptosis regulators (FIG. 13A). NCOA4-mediated ferritinophagy (the autophagic degradation of the intracellular iron storage protein ferritin) increases intracellular labile iron levels; consequently, NC (A4 deletion suppresses ferroptosis by limiting the labile iron pool [24-25]. Correspondingly, PARP inhibitor treatment increased labile iron levels in BRCA1-deficient cancer cells (FIGS. 6G and 6H), and this effect was attenuated by decreasing NCOA4 expression (FIGS. 61 and 6J); of note, decreased NCOA4 expression under PARPi treatment to the level similar to that in control cells under basal conditions (but not completely abrogated NCOA4 expression; FIG. 6I), which allowed a better investigation of whether PARPi-induced NCOA4 expression (rather than basal NCOA4 expression) plays a role in ferroptosis induced by PARPi+GPX4i combination. Furthermore, iron chelator DFO treatment or NCOA4 deletion largely abolished PARP inhibitor-induced lipid peroxidation or PARP inhibitor+GPX4 inhibitor combination-induced ferroptosis (FIGS. 6K-6N and 13B-13E), suggesting that PARP inhibitor promotes lipid peroxidation and that PARPi in combination with GPX4i trigger ferroptosis in BRCA1-deficient cancer cells mainly by inducing NCOA4 expression and enhancing intracellular labile iron levels. Notably, the increases of NCOA4 expression and iron levels were not observed in BRCA1-WT HS578T cells, while BRCA1 deletion in these cells restored PARPi-induced NCOA4 expression and iron levels (FIGS. 6O and 6P). These data reveal a regulation of NCOA4 expression by BRCA1 under PARPi treatment conditions, and partly explain why PARPi and GPX4i combination induces more potent ferroptosis in BRCA1-deficient cancer cells than in BRCA1-WT counterparts.

It was also determined that PARPi raised GPX4 levels in BRCA1-WT HS578T cells, but not in BRCA1-deficient counterparts or BRCA1-mutant HCC1937 or SUM149 cells (FIGS. 6Q and 6R). Consistently, PARPi moderately induced ferroptosis in BRCA1-mutant or -deficient cells, but not in BRCA1-WT cells (FIGS. 13F-13H); likewise, PARPi mildly induced lipid peroxidation in BRCA1-WT cells, but induced lipid peroxidation more in BRCA1-mutant or -deficient cells (FIGS. 11C and 11D). Further, blocking PARPi-induced adaptive response by reducing GPX4 expression in HS578T cells (which exhibited robust GPX4 induction under PARPi treatment; see FIG. 6Q) increased PARPi-induced ferroptosis (FIGS. 6S and 6T), whereas GPX4 overexpression in BRCA1-mutant SUM149 cells (which exhibited defective GPX4 induction after treatment with PARPi; see FIG. 6R) suppressed ferroptosis induced by PARPi or PARPi+GPX4i combination (FIGS. 6U and 6V).

Considering that proteins involved in anti-oxidant defense can often be induced by oxidative stress and that PARPi trigger reactive oxygen species (ROS) [26], it was investigated that PARPi-induced GPX4 expression most likely reflects an adaptive response to PARPi-mediated ROS stress. In support of this, it was confirmed that PARPi-induced ROS in BRCA1-WT HS578T cells (FIG. 13I) and showed that treatment with the ROS scavenger N-acetyl-1-cysteine (NAC) abolished PARPi-induced GPX4 induction in HS578T cells (FIG. 13J). Consistent with lipid peroxidation induction (FIGS. 11C and 11D), PARPi induced higher levels of ROS in BRCA1-depleted/-mutant cells than in BRCA1-WT cells (FIG. 13I). It was further examined that PARPi-induced cell death in BRCA1-deficient SUM149 cells are suppressed by NAC (FIG. 13K), suggesting that ROS contribute to PARPi-induced cell death in BRCA1-deficient cells.

Together, the data suggest a model in which BRCA1 deficiency elicits at least two cellular effects in response to PARPi treatment: (1) in WT cells, PARPi induces GPX4 expression in a ROS-BRCA1-dependent manner, as an adaptive response to antagonize PARPi-induced lipid peroxidation and ferroptosis. Consequently, BRCA1 deficiency disrupts this induction of GPX4 expression (FIG. 6W). (2) In addition, BRCA1 deficiency upregulates NCOA4 levels and NCOA4-mediated ferritinophagy in response to PARPi treatment (FIG. 6W). These effects collectively render PARPi-treated BRCA1-deficient cells highly susceptible to GPX4i-induced ferroptosis (FIG. 6W).

### Example 7: GPX4 Inhibition Overcomes PARP Inhibitor Resistance in BRCA1-Mutant Tumors

Next, mammary fat pad orthotopic injections in mice were performed (FIG. 7A) and the efficacy of PARPi (olaparib)+GPX4i (JKE-1674) combination therapy in treating BRCA1-mutant breast cancer was examined in xenograft models resistant to PARPi. Treatment with olaparib alone moderately suppressed HCC1937 xenograft tumor growth (FIGS. 7B and 14A). Treatment with JKE-1674 alone significantly reduced tumor growth, whereas the combination treatment suppressed the tumor growth even more potently; in addition, the ferroptosis inhibitor liproxstatin-1 partially restored HCC1937 xenograft tumor growth in the olaparib+JKE-1674 treatment group (FIGS. 7B and 14A), indicating that the combination therapy suppresses tumor growth at least partly by inducing ferroptosis in tumors.

Olaparib+JKE-1674 combination therapy was examined in two BRCA1-mutant patient-derived xenograft (PDX) models using two different treatment strategies. These BRCA1-mutant PDX models (PDX18_S and PDX27_S) were established from post PARPi-treated tumors from breast cancer patients with germline BRCA1 truncating mutations (c.5177_5180delGAAA for PDX18_S and c.2359 dup.G for PDX27_S) who exhibited PARPi resistance [27]. In one set of experiments, the combination of JKE-1674 with olaparib was tested and tumor samples were collected at the same endpoints for subsequent immunohistochemical (IHC) analyses. Results revealed that treatment with JKE-1674 showed efficacy in reducing tumor growth in PDX18-S and PDX27-S, and olaparib+JKE-1674 combination therapy suppressed tumor growth and overcame PARPi resistance in these PDXs (FIGS. 7C, 7D, 14B, and 14C). The suppressed tumor growth caused by the combination therapy could be restored by treatment with liproxstatin-1 (FIGS. 7C, 7D, 14B, and 14C).

To expand the applicability of the preclinical investigations, the PDX studies were repeated using another FDA-approved PARPi, talazoparib, and conducted animal survival analyses. Results showed that the application of talazoparib as a standalone treatment failed to induce a reduction in tumor growth or an extension in animal survival, confirming the PARPi resistance in these PDX models; the treatment with JKE-1674, and the combined administration of JKE-1674 and talazoparib, yielded substantial extensions in animal survival (FIGS. 7E-7H). Importantly, the extension of mean animal survival periods in the combination treatment group surpassed the expectations of a mere additive outcome, underscoring the potency of its synergistic effect (vehicle, talazoparib, JKE-1674, and JKE-1674+talazoparib combination groups exhibited mean animal survival durations of 36.5, 39, 53.5, and 97.5 days in PDX 18-S, and 23, 26, 36, and 74 days in PDX 27-S, respectively).

Two additional PDX models were studied. Firstly, to provide a comparison between PARPi-sensitive and PARPi-resistant models, a PARPi-sensitive PDX line (PDX16, which was established from a patient with BRCA1 mutation (c.5324T>G) who was responsive to PARPi treatment; see Example 8) was included. Results confirmed that PARPi (olaparib or talazoparib) almost completely suppressed tumor growth in PDX16 (FIG. 14D). Further, the treatment with JKE-1674 alone or the combination treatment exhibited minimal to moderate therapeutic effects in BRCA1-WT PIM224 PDXs (FIGS. 71 and 14E). This control demonstrates that the combination therapy does not universally work in all cancer contexts, thereby reinforcing that combination of PARPi and GPX4i targets BRCA-mutant cancers.

IHC analyses revealed that GPX4 levels in the BRCA1-WT PIM224 tumor samples were higher than those in BRCA1-mutant PDX18-S and PDX27-S samples (FIGS. 7J and 7K); mirroring the in vitro observations (FIGS. 6Q and 6R), treatment with olaparib induced GPX4 levels in BRCA1-WT PDX tumors but not in BRCA1-mutant tumors (FIGS. 7J and 7K). The decreased GPX4 levels, particularly under PARPi treatment, in PARPi-resistant BRCA1-mutant tumors might explain why these tumors were sensitive to GPX4i+PARPi combination therapy. In these PDX studies, staining for 4-HNE (FIGS. 7L and 14F), but not for either phospho-H2AX or cleaved caspase-3 (FIGS. 7M and 14G-14I), correlated with tumor growth change in different treatment groups (i.e., olaparib+JKE-1674 combination therapy increased whereas liproxstatin-1 therapy suppressed 4-HNE staining), indicating that ferroptosis induction (but not apoptosis induction or DNA damage response) underlies the therapeutic efficacy of the combination of GPX4i+PARPi in these preclinical models. Finally, these treatments did not cause any obvious toxicity or decrease the animals' weight in our animal studies (FIGS. 14J-14M), suggesting that the treatment was well tolerated in vivo.

### Discussion

The data reveals that BRCA1 has a unique dual role in regulating ferroptosis by controlling the transcription of VDAC3 and GPX4 (FIG. 7N), and that BRCA1 deficiency promotes resistance to erastin-induced ferroptosis and renders cancer cells susceptible to GPX4i-induced ferroptosis (FIG. 7O). The data further shows that GPX4i+PARPi combination therapy induced potent ferroptosis in BRCA1-deficient/-mutant cancer cells and xenograft tumors, and overcame PARPi resistance in BRCA1-mutant PDXs. The data proposes the following mechanisms to explain these findings (FIG. 7O): (1) PARPi induce lipid peroxidation by promoting NCOA4-mediated ferritinophagy in BRCA1-deficient cancer cells; (2) BRCA1 deficiency in combination with GPX4i abrogates an adaptive response to PARPi therapy to boost GPX4 expression; and (3) compared to BRCA1-WT tumors, BRCA1-mutant tumors appear to exhibit decreased GPX4 expression under PARPi treatment, rendering such tumors sensitive to GPX4 inhibition.

From a clinical standpoint, BRCA1-proficient cancers do not constitute the primary target population for PARPi treatment. Therefore, a key distinction between data provided here and previous ones lies in the findings described here of the susceptibility of BRCA1-deficient cancers to ferroptosis and combining GPX4i and PARPi for treating BRCA1-deficient cancers.

Without wishing to be bound by any one theory, the rationale behind the synergy of GPX4i and PARPi in overcoming PARPi resistance in BRCA1-mutant cancer cells relates to the low expression of GPX4 and heightened sensitivity to GPX4i-induced ferroptosis in BRCA1-mutant cancer cells. This is independent of other PARPi resistance mechanisms that may be at play. The data provides two illustrative examples (1) PARPi resistance caused by BRCA1 reversion and (2) PARP resistance caused by 53BP1 deletion. In the case of BRCA1 reversion, it is expected that BRCA1 reversion should restore GPX4 expression and promote resistance to RSL3-induced ferroptosis. However, certain BRCA1-reverted cells retained low GPX4 expression and increased sensitivity to GPX4i. BRCA1 reversion involves complex selection processes in BRCA1-deficient cells under PARPi treatment conditions and is not equivalent to restoring WT BRCA1 expression in BRCA1-deficient cells. While the exact mechanisms underlying the observed GPX4 expression patterns in certain BRCA1-reverted cells (wherein PARPi resistance is regained, yet sensitivity to GPX4i is maintained) may not be fully elucidated, this observation supports GPX4 expression as a potential biomarker for selecting patients with BRCA1 reversion mutations to benefit from GPX4i+PARPi combination therapy.

Without wishing to be bound by any one theory, BRCA1's role in ferroptosis regulation appears to be independent of its canonical function in DNA damage response and repair, but is mediated by its non-canonical function in regulating gene transcription. Importantly, the data showed that BRCA2 deficiency does not affect ferroptosis sensitivity or GPX4 and VDAC3 expression levels. The data shows that BRCA1 regulates GPX4 transcription through its BRCT domain. The interaction between BRCA1 and BRCA2 is not mediated by BRCA1's BRCT domain but instead involves the coiled-coil domain within BRCA1 [29]; therefore, BRCA2 deficiency does not affect the ability of BRCA1 to regulate GPX4 expression through its BRCT domain, explaining why the effects on GPX4 expression and ferroptosis sensitivity were not observed in the context of BRCA2 deficiency. While PARPi is used to treat patients with either BRCA1 or BRCA2 deficiency, the data here support that the PARPi+GPX4i combination therapy benefits cancer patients with BRCA1 deficiency, but not those with BRCA2 deficiency. This information supports patient selection in clinical studies with the PARPi+GPX4i combination therapy.

The data here identified BRCA1-M1775R mutant (but not BRCA1-C61G mutant) as a loss-of-function mutant in regulating GPX4 expression and GPX4i-induced ferroptosis. The C-terminal BRCT domain of BRCA1 is a phosphoprotein binding domain, and M1775R mutation is known to abolish BRCA1 interaction with other phosphoproteins [29, 30, 31]. Furthermore, the phosphorylation events in these phosphoproteins and the interaction between BRCA1's BRCT domain and phosphoproteins are often regulated by upstream stimuli (such as DNA damage and oxidative stress). Therefore, without wishing to be bound by one theory, it is possible that BRCA1 interacts with a phosphorylated transcription factor through its BRCT domain to promote GPX4 transcription, and this interaction is further regulated by PARPi-induced oxidative stress. Besides their established role in DNA repair, PARPs possess the capacity to modify histones and remodel chromatin architecture, consequently controlling gene expression [32]. The interplay between PARPs and gene transcription provides a path through which the expression of genes such as GPX4 and NCOA4 can be influenced in response to PARPi treatment.

The data in which VDAC3-KO cells exhibited remarkable resistance to ferroptosis induced by erastin at low doses but did not affect cystine starvation-induced ferroptosis support that erastin treatment at low doses (e.g., 2.5 μM) is not equivalent to cystine starvation because low-dose erastin treatment can only partially block SLC7A11-mediated cystine uptake. The data suggests that erastin-induced ferroptosis reflects a combinatorial effect of both weakening ferroptosis defense and further boosting mitochondrial activities to drive ferroptosis. Disabling this effect, as occurred in VDA (3-KO cells, weakened ferroptosis-inducing activity of erastin, explaining the remarkable resistance phenotypes in VDAC3-KO cells treated with 2.5 μM erastin (see FIG. 4D). It should be noted that erastin at very high doses (e.g., 10 μM; see FIG. 4D) can still induce cell death in VDAC3-KO cells, because of more complete blockade of SLC7A11-mediated cystine uptake at this high concentration. Furthermore, the lack of phenotypes in VDAC3-KO cells to cystine starvation-induced ferroptosis suggests that basal activity of VDAC3 is not important for this type of ferroptosis (in contrast, VDAC3 is critical for erastin-induced ferroptosis because of erastin's gain-of-function effect to promote VDAC function). The data supports that erastin-like FINs (such as IKE) would work better in ferroptosis-inducing therapy for cancer treatment than more specific SLC7A11 inhibitors.

Because BRCA1-deficient cells are much more vulnerable to ferroptosis induced by GPX4i or by GPX4i+PARPi combination, than are BRCA1-proficient cells, there is a therapeutic window allowing low-dose GPX4i to selectively kill BRCA1-deficient tumors while sparing normal tissues. GPX4i therapy at the dose of JKE-1674 provided in the data, did not show obvious toxicities in the animal studies but did reduce the growth of BRCA1-deficient tumors. The data supports clinical studies with PARPi+GPX4i combination therapy in patients with BRCA1-mutant tumors and PARPi resistance.

### Example 8: Methods

### I. Cell Culture Studies

SUM149 parental cells and clones BRCA1 with reversion mutations were provided by Dr. Mien-Chie Hung at MD Anderson Cancer Center (MDACC) and were described as previously published [28]. DLD-1 and UWB1.289 cell lines were provided by Dr. Junjie Chen at MDACC. HEY and SKOV3 cell lines were provided by Dr. Jinsong Liu at MDACC. MDA-MB-453 cells were obtained from the Cytogenetics and Cell Authentication Core at MDACC. SUM1315 cells were obtained from the BioIVT, and all other cancer cell lines were obtained from the American Type Culture Collection (ATCC). All cell lines were free of mycoplasma contamination (tested by the vendor). No cell line used in the current study has been found in the International Cell Line Authentication Committee database of commonly misidentified cell lines, based on short tandem repeat profiling performed by the vendor. UWB1.289, MCF10A, SUM149, and SUM1315 cells were cultured in medium based on the instruction of ATCC or BioIVT. All other cell lines were cultured in RPMI-1640 or Dulbecco's modified Eagle medium (DMEM) with 10% (volume/volume; v/v) fetal bovine serum and 1% (v/v) penicillin/streptomycin at 37° C. with a humidified atmosphere of 20% O₂ and 5% CO₂. For cystine-starvation experiments, cells were cultured in cystine-free media+10% (v/v) dialyzed fetal bovine serum as previously described47. Mutation information for the cell lines is listed in Table 1.

TABLE 1
Relevant gene mutation information for the cell lines and xenografts.

| | BRCA1 | p53 | KRAS | KEAP1 | STK11 | NFE2L2 |
|---|---|---|---|---|---|---|
| PDX 18-S | c.5177_5180del GAAA | c.G524A | WT | WT | WI | WT |
| PDX 27-S | c.2359 dup.G. | c.C1024T | WT | WT | WT | WT |
| PDX | WT | WT | WT | WT | WT | WT |
| PIM224 | | | | | | |
| HS578T | WT | p.V157F | WT | WT | WT | WT |
| MCF10A | WT | WT | WT | WT | WT | WT |
| HCC1806 | WT | p.T256fs | WT | WT | WT | WT |
| MDA-MB- | WT | Ex10-11del | p.Gly13Asp | WT | WT | WT |
| 453 | | | (c.38G > A) | | | |
| MDA-MB- | WT | p.R273H | WT | WT | WT | WT |
| 468 | | | | | | |
| SUM149 | c.2169delT | p.M237I | WT | WT | WT | WT |
| HCC1937 | c.5266dupC | p.R306* | WT | WT | WT | WT |
| SUM1315 | c.68_69delAG | p.C135F | WT | WT | WT | WT |
| HCC1395 | c.5251C > T | p.R175H | WT | WT | WT | WT |
| HT1080 | WT | WT | WT | WT | WT | WT |
| HEY | WT | WT | p.G12D | WT | WT | WT |
| SKOV3 | WT | p.S90fs | WT | WT | WT | WT |
| RPMI7951 | WT | p.S166* | WT | WT | WT | WT |

### II. Constructs and Reagents

GPX4 expression plasmids were described previously [21]. BRCA1-WT, BRCA1-C61G mutant, and BRCA1-M1775R mutant expression plasmids were obtained from Dr. Junjie Chen at MDACC [33]. VDAC2 and VDAC3 cDNAs were obtained from the Functional Genomics Core Facility at MDACC and subsequently cloned into the lentivirus vector pLV-EF1a-IRES-Blast.

FINs included RSL3 (Selleckchem), ML210 (Selleckchem), ML162 (Cayman Chemical), JKE-1674 (MedChemExpress), erastin (Selleckchem), imidazole ketone erastin (Selleckchem), and sulfasalazine (Sigma-Aldrich); PARPi included olaparib (Selleckchem) and niraparib (Selleckchem); cell death inhibitors included ferrostatin-1 (Selleckchem), liproxstatin-1 (Cayman Chemical), and Z-VAD-FMK (Selleckchem); iron chelator and antioxidants included DFO (Selleckchem), TEMPO (Sigma-Aldrich), and MitoTEMPO (Sigma-Aldrich).

### III. CRISPR-Cas9—Mediated Gene Knockout and Overexpression Cell Line Generation

Knockout of BRCA1, BRCA2, VDAC3, GPX4, ECAD, 53BP1, and NCOA4 in human cell lines was performed using single guide RNAs (sgRNAs) and the CRISPR-Cas9 expression system as previously described [34, 35]. Briefly, sgRNAs were cloned into the lentiviral LentiGuide vector. The sequences of sgRNAs used in the current study are listed in SEQ ID NOs 1-23. LentiGuide clones were transfected into HEK293T cells with a psPAX2 packaging plasmid and pMD2.G-expressing plasmid. Cells were infected with lentivirus with 0.8 μg/mL polybrene and selected with puromycin (1 μg/mL, InvivoGen) or blasticidin (2 μg/mL, InvivoGen), followed by Western blot analysis to confirm target gene deletion. To maintain GPX4 knockout cells, we added 1 μM ferrostatin-1 into the media.

Stable cell lines overexpressing target gene constructs were generated as described previously [36, 37]. Briefly, HEK293T cells were transfected with either empty vector or target gene constructs, together with the psPAX.2 and pMD2.G third-generation lentiviral packaging system using 0.8 μg/mL polybrene. After 48 hours, lentivirus particles in the medium were collected and filtered, and then the target cell lines were infected, followed by puromycin selection to obtain stable cell lines with successful transduction.

### IV. Cell Viability and Cell Death Assay

Viable cells were measured using Cell Counting Kit-8 (CCK-8, Dojindo) as previously described [38, 39]. Briefly, cells were seeded onto 96-well plates and subsequently treated with indicated agents. Next, cells were exposed to 10 μL of CCK-8 reagent (100 μL of medium per well) for 1 hour at 37° C. with 5% CO₂ in an incubator. The absorbance at a wavelength of 450 nm was determined using a FLUOstar Omega microplate reader (BMG Labtech). The combination effects were assessed with Bliss independence model and the score is defined as “observed combination effect” minus “expected additive effect”. The synergy indicates that the “observed combination effect” exceeds the “expected additive effect”.

Cell death was measured by propidium iodide (Roche) staining using a flow cytometer, as previously described [40-42]. Briefly, cells were seeded onto 6- or 12-well plates and subsequently treated with indicated agents. For GPX4 knockout cells, ferrostatin-1 was removed from the culture media. Next, cells were collected (including floating dead cells) and stained with 5 μg/mL propidium iodide. The percentage of the propidium iodide-positive dead cells was determined using the flow cytometer BD Accuri C6 (BD Biosciences) or Attune NXT Flow Cytometer (ThermoFisher) with an FL2 detector. A minimum of 5,000 single cells were analyzed per well.

### V. Lipid Peroxidation and ROS Measurement

As previously described [21, 41, 43, 44] C11-BODIPY 581/591 (Invitrogen) was used for lipid peroxidation, MitoPerOx (Abcam) for mitochondrial lipid peroxidation, and CM-H2DCFDA (ThermoFisher) for ROS measurements. Briefly, cells were seeded on 6- or 12-well plates and subsequently treated with indicated agents. After staining with 2.5 μM C11-BODIPY 581/591, 2.5 μM MitoPerOx, or 4 μM CM-H2DCFDA for 20 minutes, cells were analyzed using the flow cytometer BD Accuri C6 (BD Biosciences) or Attune NxT Flow Cytometer (ThermoFisher) with a 488-nm laser on an FL1 detector. A minimum of 5,000 single cells were analyzed per well.

### VI. Labile Iron Pool Measurement

Labile iron pool was measured according to methods described previously [24, 45]. Briefly, the treated cells were incubated with 0.05 μM calcein-AM (C3099, Invitrogen) for 15 minutes at 37° C. Subsequently, cells were washed twice with phosphate-buffered saline (PBS) and then left untreated or incubated with DFO for 1 hour at 37° C. After being washed with PBS, cells were analyzed using Attune NxT Flow Cytometer (ThermoFisher) with a 488-nm laser on an FL1 detector. The difference in the cellular mean fluorescence with and without DFO incubation reflects the amount of labile iron pool.

### VII. Glutathione Measurement

Glutathione level measurements were performed as previously described [36, 46]. Briefly, cells were seeded onto 96-well plates and subsequently treated with erastin. Next, the media containing erastin was replaced with 100 μL of prepared 1×GSH-Glo Reagent and incubated for 30 minutes. Then, 100 μL of reconstituted Luciferin Detection Reagent was added, gently mixed on a plate shaker, and incubated for 20 minutes. Relative glutathione levels were assessed by luminescent signals using a Gen5 Microplate reader (BIOTEK). Results were normalized to cell viability.

VIII. Quantitative PCR with Reverse Transcription

Quantitative reverse transcription PCR was performed as previously described [47, 48]. Briefly, total RNA was extracted using TRIzol reagent (Invitrogen), and reverse transcription was performed using iScript Reverse Transcription Supermix (Biorad). SYBR GreenER qPCR SuperMix Universal (Invitrogen) was used for quantitative PCR in triplicate, with samples run on a Stratagene MX3000P qPCR system. The threshold cycle (Ct) values for each gene were normalized to those of β-actin, and the 2-ΔΔCt method was used for quantitative analysis. The primer sequences are listed as SEQ ID NOs 24-31.

### IX. Immunoprecipitation and Western Blot Analysis

Cell pellets were lysed using immunoprecipitation lysis buffer (Fisher Scientific) or NP-40 buffer containing complete mini protease inhibitors (Roche) [49]. Protein concentrations were detected by a Bicinchoninic Acid Protein Assay (Thermo Scientific) using a FLUOstar Omega microplate reader (BMG Labtech). Immunoprecipitation and Western blot analysis were conducted as previously described [50, 51]. The primary antibodies and concentrations used in the current study included the followed: GPX4 (1:1,000, MAB5457, R&D systems), BRCA1 (1:1,000, 14823, Cell Signaling), BRCA2 (1:1,000, MAB2476-SP, R&D Systems), DHODH (1:1,000, 14877-1-AP, Proteintech), ACSL4 (1:1,000, sc-271800, Santa Cruz), FSP1 (1:1,000, 20886-1-AP, Proteintech), VDAC1 (1:1,000, 66345-1-Ig, Proteintech), VDAC2 (1:1,000, 66388-1-Ig, Proteintech), VDAC3 (1:1,000, 55260-1-AP, Proteintech), SLC7A11 (1:1,000, 12691, Cell Signaling), NCOA4 (1:1,000, 66849, Cell Signaling), vinculin (1:10000; V4505, Sigma), β-actin (1:1,000, 3700, Cell Signaling), LC3B (1:5000, 3868, Cell Signaling), 53BP1 (1:5000, NB100-304, Novus Biologicals), E-Cadherin (1:1000, 3195, Cell Signaling), and tubulin (1:5000, 2144, Cell Signaling).

### X. Luciferase Reporter Assay

Luciferase reporter assays were performed as previously described [36]. The GPX4 promoter was amplified using genomic DNA extracted from 293T cells and cloned into pGL3 luciferase reporter vectors. The luciferase reporter assay was conducted using the Dual-Luciferase Reporter Assay System (Promega, E1910 and E1960) according to the manufacturer's instructions. Briefly, 293T cells were transfected with each plasmid for 48 hours, washed with PBS, and lysed for 15 minutes at room temperature. Cell lysates were transferred to a 96-well plate for subsequent luciferase activity measurement. The sample was then placed in the luminometer and read.

### XI. ChIP Assays

ChIP assays were essentially performed using the SimpleChIP Enzymatic Chromatin IP Kit (Cell Signaling, #9003) as previously described [52, 53]. Cells were fixed with formaldehyde (1% final volume concentration) for 10 minutes at room temperature. Fixation was stopped by adding glycine and incubating for 5 minutes at room temperature. Chromatin (10 μg) was incubated overnight with 10 μg of BRCA1 (1:1,000, 22362-1-AP, Proteintech) after digestion. Antibody-protein complex was captured with ChIP-Grade Protein G Magnetic Beads (Cell Signaling, 9006). ChIP DNA was analyzed by quantitative PCR with SYBR GreenER qPCR SuperMix Universal (Invitrogen) in a Stratagene MX3000P qPCR system using the primers as listed in SEQ ID NOs 32-39.

### XII. Orthotopic and Subcutaneous Xenograft Model

Female 4- to 6-week-old athymic nude mice (Foxn1^{nu}/Foxn1^{nu}) and NOD scid gamma (NSG) mice were obtained from the Experimental Radiation Oncology Breeding Core Facility at MD Anderson. All mice were maintained under specific pathogen-free housing in the Animal Care Facility in the Department of Veterinary Medicine and Surgery at MD Anderson. Rodent housing conditions used in the current study were as follows: temperature set point 72° F., high limit 74° F., low limit 70° F.; humidity set point 45%, high limit 55%, low limit 40%; light cycle 12 hours light-dark. The study is compliant with all relevant ethical regulations regarding animal research.

PDX lines (including BRCA1-WT line PIM224, and BRCA1-mutant lines 16, 18-S, and 27-S) were obtained from Dr. Helen Piwnica-Worms at MDACC and maintained/expanded on NSG mice as previously described [54]. The BRCA1-mutant tumors used to create the PDX models were obtained in alignment with a phase II neoadjuvant clinical trial (NCT03499353) conducted at MDACC [27]. The objective of the trial was to evaluate the pathologic response and toxicity to single-agent talazoparib for 6 months in 20 patients with stage I to III breast cancer and who were gBRCA1/2-positive before definitive surgery. Upon enrollment, patients were biopsied to provide tumor material for implantation into mice for PDX establishment. Patients were then administered a single oral dose of talazoparib once per day for 6 cycles (each cycle was 28 days), followed by an additional biopsy for PDX establishment and surgery where residual cancer burden (RCB) was determined. Patients with either pathological complete response (pCR) or RCB-I were considered sensitive, while patients with RCB-II or RCB-III were considered resistant to talazoparib. RCB index was classified as: RCB-I (minimal burden), RCB-II (moderate burden) or RCB-III (extensive burden). PDX-16, PDX18-S, and PDX27-S were from patients with RCB—I, RCB-III, RCB-II, respectively.

Orthotopic implantation was conducted as previously described [54]. For cell line-derived orthotopic xenograft models, 5×106 HCC1937 cells were resuspended in 50% volume Matrigel (Invitrogen)+50% volume medium and implanted into the fourth inguinal mammary fat pad of nude mice. For orthotopic PDX models, PDX tumors were harvested and dissociated into single cells by mechanical mincing and digestion69; 1×106 digested PDX 16, 18-S, 27-S, or PIM224 tumor cells were resuspended in 50% volume Matrigel (Invitrogen)+50% volume medium and implanted into the fourth inguinal mammary fat pad of NSG mice. For cell line-derived subcutaneous xenograft models, 6×106 Cas9 control or BRCA1-single-guide RNA (sgRNA)-infected SKOV3 cells were resuspended in 50% volume Matrigel (Invitrogen)+50% volume medium and injected into nude mice subcutaneously.

In all these animal studies, mice were randomized to different treatment groups when tumors reached 50-100 mm3. Olaparib (MedChemExpress) was dissolved in dimethyl sulfoxide and diluted in 30% volume PEG 300+70% volume PBS. JKE-1674 used in mouse administration was synthesized by Institute for Applied Cancer Science at MDACC. JKE-1674, talazoparib (Selleckchem), and liproxstatin-1 (Cayman Chemical) were dissolved in dimethyl sulfoxide and diluted in PBS. JKE-1674 (25 mg/kg), olaparib (40 mg/kg), and talazoparib (0.333 mg/kg) were administered to mice every 2 days. Liproxstatin-1 (10 mg/kg) was administered to mice every day. Treatment with olaparib, JKE-1674, talazoparib, or liproxstatin-1 was continued until the endpoint as indicated in the corresponding figures. The volume of tumors was measured with a caliper one to three times per week until the endpoint and calculated according to the following equation: volume=length×width2× ½. Survival was assessed using the Kaplan-Meier survival curve and differences in survival were calculated by log-rank test. Relevant gene mutation information for the tumors used in the xenograft models is listed in Table 1.

### XII. Histology and Immunohistochemistry

Histologic analysis and immunohistochemistry staining were performed as previously described [56, 57]. Briefly, tumor tissues were collected, immediately fixed in 10% neutral-buffered formalin (ThermoFisher) overnight, and stored in 70% ethanol at 4° C. The tissues were dehydrated and embedded in paraffin by the Research Histology Core Laboratory at MDACC according to standard protocols. Samples were sectioned at a thickness of 5 μm and subjected to hematoxylin and eosin or immunohistochemistry staining. The primary antibodies and concentrations used for immunohistochemistry included the following: anti-4-hydroxynonenal (4-HNE) (1:300, Abcam, ab46545), anti-phospho-H2AX (1:500, EMD Millipore, Cat #05-636), anti-GPX4 (1:150, Novus Biologicals, NBP2-54979), RAD51 (1:200, Abcam, ab133534), and anti-cleaved caspase-3 (1:500, Cell Signaling, 9661s). Staining was performed using the Vectastain elite ABC kit and DAB peroxidase substrate kit (Vector laboratories). Images were randomly acquired at 200× or 400× magnification using an Olympus BX43 microscope, and an immunoreactive score system was used to semiquantitatively assess the level of staining [58].

### XIV. Statistics and Reproducibility

Statistical analyses were performed with GraphPad Prism 8 software using unpaired Student/tests, two-way ANOVA, or log-rank test. The results of cell-based experiments were collected from at least three independent replicates. The results of animal-based experiments were collected from at least six tumors in each group. Data are presented as means±standard deviation. Statistical significance levels (P values) are indicated as follows: *P<0.05; **P<0.01; ***P<0.001; ****P<0.0001; NS, not significant. No statistical methods were used to predetermine sample size. Sample size was determined according to our experience as well as literature reporting in terms of the specific experiment.

### REFERENCES

1. Basu, A., et al., An interactive resource to identify cancer genetic and lineage dependencies targeted by small molecules. Cell, 2013. 154(5): p. 1151-1161.
2. Roy, R., J. Chun, and S. N. Powell, BRCA1 and BRCA2: different roles in a common pathway of genome protection. Nat Rev Cancer, 2011. 12(1): p. 68-78.
3. Bryant, H. E., et al., Specific killing of BRCA2-deficient tumours with inhibitors of poly (ADP-ribose) polymerase. Nature, 2005. 434(7035): p. 913-7.
4. Farmer, H., et al., Targeting the DNA repair defect in BRCA mutant cells as a therapeutic strategy. Nature, 2005. 434(7035): p. 917-21.
5. Gao, M., et al., Role of Mitochondria in Ferroptosis. Mol Cell, 2019. 73 (2): p. 354-363 e3.
6. Gan, B., Mitochondrial regulation of ferroptosis. J Cell Biol, 2021. 220 (9).
7. Trnka, J., et al., A mitochondria-targeted nitroxide is reduced to its hydroxylamine by ubiquinol in mitochondria. Free Radic Biol Med, 2008. 44(7): p. 1406-19.
8. Bae, I., et al., BRCA1 induces antioxidant gene expression and resistance to oxidative stress. Cancer Res, 2004. 64(21): p. 7893-909.
9. Gorrini, C., et al., BRCA1 interacts with NRF2 to regulate antioxidant signaling and cell survival. J Exp Med, 2013. 210(8): p. 1529-44.
10. Xu, P., et al., Breast cancer susceptibility protein 1 (BRCA1) rescues neurons from cerebral ischemia reperfusion injury through NRF2-mediated antioxidant pathway. Redox Biol, 2018. 18: p. 158-172.
11. Sykiotis, G. P. and D. Bohmann, Stress-activated cap'n'collar transcription factors in aging and human disease. Sci Signal, 2010. 3(112): p. re3.
12. Rojo de la Vega, M., E. Chapman, and D. D. Zhang, NRF2 and the Hallmarks of Cancer. Cancer Cell, 2018.
13. Marcar, L., et al., Acquired Resistance of EGFR-Mutated Lung Cancer to Tyrosine Kinase Inhibitor Treatment Promotes PARP Inhibitor Sensitivity. Cell Rep, 2019. 27(12): p. 3422-3432 e4.
14. Hassannia, B., et al., Nano-targeted induction of dual ferroptotic mechanisms eradicates high-risk neuroblastoma. J Clin Invest, 2018. 128 (8): p. 3341-3355.
15. Feng, C., et al., Identification of the Viral Determinant of Hypovirulence and Host Range in Sclerotiniaceae of a Genomovirus Reconstructed from the Plant Metagenome. J Virol., 2021. 95(17): e0026421.
16. Wu, J., et al. (2019). Intercellular interaction dictates cancer cell ferroptosis via NF2-YAP signalling. Nature 572, 402-406. 10.1038/s41586-019-1426-6.
17. Lamber, E. P., et al. (2010). BRCA1 represses amphiregulin gene expression. Cancer Res 70, 996-1005. 10.1158/0008-5472.CAN-09-2842.
18. Dixon, S. J., et al. (2012). Ferroptosis: an iron-dependent form of nonapoptotic cell death. Cell 149, 1060-1072. 10.1016/j.cell.2012.03.042.
19. Yagoda, N., et al. (2007). RAS-RAF-MEK-dependent oxidative cell death involving voltage-dependent anion channels. Nature 447, 864-868. 10.1038/nature05859.
20. Burrows, A. E., et al. Polybromo-associated BRG1-associated factor components BRD7 and BAF180 are critical regulators of p53 required for induction of replicative senescence. Proc Natl Acad Sci USA 107, 14280-14285.
21. Mao, C., et al. (2021). DHODH-mediated ferroptosis defence is a targetable vulnerability in cancer. Nature 593, 586-590. 10.1038/s41586-021-03539-7.
22. Dias, M. P., et al. (2021). Understanding and overcoming resistance to PARP inhibitors in cancer therapy. Nat Rev Clin Oncol 18, 773-791. 10.1038/s41571-021-00532-x.
23. Noordermeer, S. M., and van Attikum, H. (2019). PARP Inhibitor
Resistance: A Tug-of-War in BRCA-Mutated Cells. Trends in cell biology 29, 820-834. 10.1016/j.tcb.2019.07.008.
24. Gao, M., et al. (2016). Ferroptosis is an autophagic cell death process. Cell research 26, 1021-1032. 10.1038/cr.2016.95.
25. Hou, W., et al. (2016). Autophagy promotes ferroptosis by degradation of ferritin. Autophagy 12, 1425-1428. 10.1080/15548627.2016.1187366.
26. Marcar, L., et al. (2019). Acquired Resistance of EGFR-Mutated Lung Cancer to Tyrosine Kinase Inhibitor Treatment Promotes PARP Inhibitor Sensitivity. Cell Rep 27, 3422-3432 e3424. 10.1016/j.celrep.2019.05.058.
27. Litton, J. K., et al. (2020). Neoadjuvant Talazoparib for Patients With Operable Breast Cancer With a Germline BRCA Pathogenic Variant. J Clin Oncol 38, 388-394. 10.1200/JCO.19.01304.
28. Shih, D. J. H., et al. (2022). Exploiting induced vulnerability to overcome PARPi resistance and clonal heterogeneity in BRCA mutant triple-negative inflammatory breast cancer. Am J Cancer Res 12, 337-354.
29. Huen, M. S., Sy, S. M., and Chen, J. (2010). BRCA1 and its toolbox for the maintenance of genome integrity. Nat Rev Mol Cell Biol 11, 138-148. 10.1038/nrm2831.
30. Manke, I. A., Lowery, D. M., Nguyen, A., and Yaffe, M. B. (2003). BRCT repeats as phosphopeptide-binding modules involved in protein targeting. Science 302, 636-639. 10.1126/science. 1088877.
31. Yu, X., Chini, C. C., He, M., Mer, G., and Chen, J. (2003). The BRCT domain is a phospho-protein binding domain. Science 302, 639-642. 10.1126/science.1088753.
32. Gupte, R., Liu, Z., and Kraus, W. L. (2017). PARPs and ADP-ribosylation: recent advances linking molecular functions to biological outcomes. Genes Dev 31, 101-126. 10.1101/gad.291518.116.
33. Feng, L., Li, N., Li, Y., Wang, J., Gao, M., Wang, W., and Chen, J. (2015). Cell cycle-dependent inhibition of 53BP1 signaling by BRCA1. Cell Discov 1, 15019. 10.1038/celldisc.2015.19.
34. Lee, H., Zandkarimi, F., Zhang, Y., Meena, J. K., Kim, J., Zhuang, L., Tyagi, S., Ma, L., Westbrook, T. F., Steinberg, G. R., et al. (2020). Energy-stress-mediated AMPK activation inhibits ferroptosis. Nat Cell Biol 22, 225-234. 10.1038/s41556-020-0461-8.
35. Wu, S., Mao, C., Kondiparthi, L., Poyurovsky, M. V., Olszewski, K., and Gan, B. (2022). A ferroptosis defense mechanism mediated by glycerol-3-phosphate dehydrogenase 2 in mitochondria. Proc Natl Acad Sci USA 119, e2121987119. 10.1073/pnas.2121987119.
36. Zhang, Y., Shi, J., Liu, X., Feng, L., Gong, Z., Koppula, P., Sirohi, K., Li, X., Wei, Y., Lee, H., et al. (2018). BAP1 links metabolic regulation of ferroptosis to tumour suppression. Nat Cell Biol 20, 1181-1192. 10.1038/s41556-018-0178-0.
37. Koppula, P., Lei, G., Zhang, Y., Yan, Y., Mao, C., Kondiparthi, L., Shi, J., Liu, X., Horbath, A., Das, M., et al. (2022). A targetable CoQ-FSP1 axis drives ferroptosis—and radiation-resistance in KEAP1 inactive lung cancers. Nature communications/3, 2206. 10.1038/s41467-022-29905-1.
38. Zhang, Y., Swanda, R. V., Nie, L., Liu, X., Wang, C., Lee, H., Lei, G., Mao, C., Koppula, P., Cheng, W., et al. (2021). mTORC1 couples cyst(e)ine availability with GPX4 protein synthesis and ferroptosis regulation. Nat Commun 12, 1589. 10.1038/s41467-021-21841-w.
39. Koppula, P., Olszewski, K., Zhang, Y., Kondiparthi, L., Liu, X., Lei, G., Das, M., Fang, B., Poyurovsky, M. V., and Gan, B. (2021). KEAP1 deficiency drives glucose dependency and sensitizes lung cancer cells and tumors to GLUT inhibition. iScience 24, 102649. 10.1016/j.isci.2021.102649.
40. Liu, X., and Gan, B. (2016). lncRNA NBR2 modulates cancer cell sensitivity to phenformin through GLUT1. Cell Cycle 15, 3471-3481. 10.1080/15384101.2016.1249545.
41. Koppula, P., Zhang, Y., Shi, J., Li, W., and Gan, B. (2017). The glutamate cystine antiporter SLC7A11/xCT enhances cancer cell dependency on glucose by exporting glutamate. J Biol Chem 292, 14240-14249. 10.1074/jbc. M117.798405.
42. Xiao, Z. D., Han, L., Lee, H., Zhuang, L., Zhang, Y., Baddour, J., Nagrath, D., Wood, C. G., Gu, J., Wu, X., et al. (2017). Energy stress-induced lncRNA FILNCI represses c-Myc-mediated energy metabolism and inhibits renal tumor development. Nat Commun 8, 783. 10.1038/s41467-017-00902-z.
43. Lei, G., Zhang, Y., Koppula, P., Liu, X., Zhang, J., Lin, S. H., Ajani, J. A., Xiao, Q., Liao, Z., Wang, H., and Gan, B. (2020). The role of ferroptosis in ionizing radiation-induced cell death and tumor suppression. Cell Res 30, 146-162. 10.1038/s41422-019-0263-3.
44. Lei, G., Zhang, Y., Hong, T., Zhang, X., Liu, X., Mao, C., Yan, Y., Koppula, P., Cheng, W., Sood, A. K., et al. (2021). Ferroptosis as a mechanism to mediate p53 function in tumor radiosensitivity. Oncogene 40, 3533-3547. 10.1038/s41388-021-01790-w.
45. Prus, E., and Fibach, E. (2008). Flow cytometry measurement of the labile iron pool in human hematopoietic cells. Cytometry A 73, 22-27. 10.1002/cyto.a.20491.
46. Yan, Y., Teng, H., Hang, Q., Kondiparthi, L., Lei, G., Horbath, A., Liu, X., Mao, C., Wu, S., Zhuang, L., et al. (2023). SLC7A11 expression level dictates differential responses to oxidative stress in cancer cells. Nature communications 14, 3673. 10.1038/s41467-023-39401-9.
47. Lee, H., Dai, F., Zhuang, L., Xiao, Z. D., Kim, J., Zhang, Y., Ma, L., You, M. J., Wang, Z., and Gan, B. (2016). BAF180 regulates cellular senescence and hematopoietic stem cell homeostasis through p21. Oncotarget 7, 19134-19146. 10.18632/oncotarget.8102.
48. Zhang, Y., Shi, J., Liu, X., Xiao, Z., Lei, G., Lee, H., Koppula, P., Cheng, W., Mao, C., Zhuang, L., et al. (2020). H2A Monoubiquitination Links Glucose Availability to Epigenetic Regulation of the Endoplasmic Reticulum Stress Response and Cancer Cell Death. Cancer Res 80, 2243-2256. 10.1158/0008-5472.CAN-19-3580.
49. Lin, A., Yao, J., Zhuang, L., Wang, D., Han, J., Lam, E. W., Network, T. R., and Gan, B. (2014). The FoxO-BNIP3 axis exerts a unique regulation of mTORC1 and cell survival under energy stress. Oncogene 33, 3183-3194. 10.1038/one.2013.273.
50. Gan, B., Melkoumian, Z. K., Wu, X., Guan, K. L., and Guan, J. L. (2005). Identification of FIP200 interaction with the TSC1-TSC2 complex and its role in regulation of cell size control. J Cell Biol 170, 379-389.
51. Gan, B., Yoo, Y., and Guan, J. L. (2006). Association of focal adhesion kinase with tuberous sclerosis complex 2 in the regulation of s6 kinase activation and cell growth. J Biol Chem 281, 37321-37329.
52. Gan, B., Lim, C., Chu, G., Hua, S., Ding, Z., Collins, M., Hu, J., Jiang, S., Fletcher-Sananikone, E., Zhuang, L., et al. (2010). FoxOs enforce a progression checkpoint to constrain mTORC1-activated renal tumorigenesis. Cancer Cell 18, 472-484. S1535-6108 (10) 00421-6 [pii] 10.1016/j.ccr.2010.10.019.
53. Lin, A., Piao, H. L., Zhuang, L., Sarbassov dos, D., Ma, L., and Gan, B. (2014). FoxO transcription factors promote AKT Ser473 phosphorylation and renal tumor growth in response to pharmacologic inhibition of the PI3K-AKT pathway. Cancer Res 74, 1682-1693. 10.1158/0008-5472.CAN-13-1729.
54. Echeverria, G. V., Ge, Z., Seth, S., Zhang, X., Jeter-Jones, S., Zhou, X., Cai, S., Tu, Y., McCoy, A., Peoples, M., et al. (2019). Resistance to neoadjuvant chemotherapy in triple-negative breast cancer mediated by a reversible drug-tolerant state. Sci Transl Med/1. 10.1126/scitranslmed.aav0936.
55. Liedtke, C., Mazouni, C., Hess, K. R., Andre, F., Tordai, A., Mejia, J. A., Symmans, W. F., Gonzalez-Angulo, A. M., Hennessy, B., Green, M., et al. (2008). Response to neoadjuvant therapy and long-term survival in patients with triple-negative breast cancer. J Clin Oncol 26, 1275-1281. 10.1200/JCO.2007.14.4147.
56. Gan, B., Peng, X., Nagy, T., Alcaraz, A., Gu, H., and Guan, J. L. (2006). Role of FIP200 in cardiac and liver development and its regulation of TNFalpha and TSC-mTOR signaling pathways. J Cell Biol 175, 121-133. jcb.200604129 [pii] 10.1083/jcb.200604129.
57. Liu, X., Olszewski, K., Zhang, Y., Lim, E. W., Shi, J., Zhang, X., Zhang, J., Lee, H., Koppula, P., Lei, G., et al. (2020). Cystine transporter regulation of pentose phosphate pathway dependency and disulfide stress exposes a targetable metabolic vulnerability in cancer. Nat Cell Biol 22, 476-486. 10.1038/s41556-020-0496-x.
58. Fedchenko, N., and Reifenrath, J. (2014). Different approaches for interpretation and reporting of immunohistochemistry analysis results in the bone tissue—a review. Diagn Pathol 9, 221. 10.1186/s13000-014-0221-9.

Having now fully described this invention, it will be understood by those of ordinary skill in the art that the same can be performed within a wide and equivalent range of conditions, formulations, and other parameters without affecting the scope of the invention or any aspect thereof.

Other aspects of the present disclosure will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A method of treating a BRCA1 deficient cancer in a subject in need thereof, comprising administering to the subject a glutathione peroxidase 4 (GPX4) inhibitor and a poly (ADP-ribose) polymerase (PARP) inhibitor.

2. The method of claim 1, wherein the subject has a BRCA1 mutation.

3. The method of any one of claim 1 or 2, wherein the subject does not have a BRCA2 deficiency.

4. The method of any one of claims 1 to 3, wherein the subject does not have a BRCA2 mutation.

5. The method of any one of claims 1 to 4, wherein the GPX4 inhibitor is selected from the group consisting of RSL3, ML162, ML210, JKE-1674, withaferin A, and a combination thereof.

6. The method of any one of claims 1 to 5, wherein the PARP inhibitor is selected from the group consisting of olaparib, niraparib, veliparib, talazoparib, rucaparib, fluzoparib, and a combination thereof.

7. The method of any one of claims 1 to 6, wherein the GPX4 inhibitor is JKE-1674, and the PARP inhibitor is olaparib.

8. The method of any one of claims 1 to 6, wherein the GPX4 inhibitor is withaferin A, and the PARP inhibitor is olaparib.

9. The method of any one of claims 1 to 6, wherein the GPX4 inhibitor is RSL3, and the PARP inhibitor is olaparib.

10. The method of any one of claims 1 to 6, wherein the GPX4 inhibitor is RSL3, and the PARP inhibitor is niraparib.

11. The method of any one of claims 1 to 10, wherein the BRCA1 deficient cancer is a tumor.

12. The method of claim 11, wherein the tumor is a carcinoma.

13. The method of any one of claims 1 to 12, wherein the BRCA1 deficient cancer is selected from the group consisting of breast cancer, ovarian cancer, colon cancer, pancreatic cancer, and prostate cancer.

14. The method of any one of claims 1 to 13, wherein the BRCA1 deficient cancer is breast cancer.

15. The method of any one of claims 1 to 14, wherein the BRCA1 deficient cancer has low GPX4 expression relative to a non-deficient BRCA1 cancer.

16. The method of any one of claims 1 to 15, wherein the BRCA1 deficient cancer is not deficient of 53BP1.

17. The method of any one of claims 1 to 15, wherein the BRCA1 deficient cancer is deficient of 53BP1.

18. The method of any one of claims 1 to 15, wherein the GPX4 inhibitor is administered prior to administration of the PARP inhibitor.

19. The method of any one of claims 1 to 15, wherein the GPX4 inhibitor is administered after administration of the PARP inhibitor.

20. The method of any one of claims 1 to 15, wherein the GPX4 inhibitor and the PARP inhibitor are administered simultaneously.

21. The method of any one of claims 1 to 15, wherein the GPX4 inhibitor and the PARP inhibitor are administered in the same composition.

22. The method of any one of claims 1 to 15, wherein the GPX4 inhibitor and the PARP inhibitor are administered in different compositions.

23. The method of any one of claims 1 to 15, wherein the administration induces ferroptosis.

24. The method of any one of claims 1 to 21, wherein the administration induces cancer cell death and/or reduces cancer cell growth in the subject.

25. The method of any one of claims 1 to 24, wherein the subject is a human.

26. A method of treating a cancer in a subject in need thereof, comprising identifying whether the subject has a BRCA1 deficiency and administering to the subject having a BRCA1 deficiency a GPX4 inhibitor and a PARP inhibitor.

27. A method of selecting a subject afflicted with a cancer as suitable for treatment with a GPX4 inhibitor and a PARP inhibitor, the method comprising identifying the subject as having a BRCA1 deficiency and treating the subject with a GPX4 inhibitor and a PARP inhibitor.

28. The method of claim 26 or 27, wherein identifying whether the subject has decreased expression of BRCA1 comprises obtaining a cancer sample from the subject and analyzing the sample for the BRCA1 expression level.

29. A method of selecting a subject afflicted with a cancer as suitable for treatment with a GPX4 inhibitor and a PARP inhibitor, the method comprising identifying the subject as having a BRCA1 mutation and treating the subject with a GPX4 inhibitor and a PARP inhibitor.

30. The method of claim 29, wherein identifying whether the subject has a BRCA1 mutation comprises obtaining a cancer sample from the subject and analyzing the BRCA1 mutation status in the sample.

31. The method of any one of claims 26 to 30, wherein the subject does not have a BRCA2 deficiency.

32. The method of any one of claims 26 to 31, wherein the subject does not have a BRCA2 mutation.

33. A method of inducing ferroptosis in a BRCA1-deficient cancer cell, comprising contacting the cell with a GPX4 inhibitor and a PARP inhibitor.

34. The method of claim 33, wherein the cancer cell is in a tumor.

35. The method of claim 34, wherein the tumor is in a human.

36. The method of any one of claims 33 to 35, wherein the ferroptosis induction is increased compared to use of a GPX4 inhibitor alone or a PARP inhibitor alone.

37. The method of any one of claims 33 to 36, wherein cell death is increased compared to use of a GPX4 inhibitor alone or a PARP inhibitor alone.

38. The method of any one of claims 33 to 37, wherein cell viability is reduced compared to use of a GPX4 inhibitor alone or a PARP inhibitor alone.

39. The method of any one of claims 33 to 38, wherein lipid peroxidation in the cell is increased compared to use of a GPX4 inhibitor alone or a PARP inhibitor alone.

40. The method of any one of claims 26 to 39 wherein the GPX4 inhibitor is selected from the group consisting of RSL3, ML162, ML210, JKE-1674, withaferin A, and a combination thereof.

41. The method of any one of claims 26 to 40, wherein the PARP inhibitor is selected from the group consisting of olaparib, niraparib, veliparib, talazoparib, rucaparib, fluzoparib, and a combination thereof.

42. The method of any one of claims 26 to 41, wherein the GPX4 inhibitor is JKE-1674, and the PARP inhibitor is olaparib.

43. The method of any one of claims 26 to 41, wherein the GPX4 inhibitor is withaferin A, and the PARP inhibitor is olaparib.

44. The method of any one of claims 26 to 41, wherein the GPX4 inhibitor is RSL3, and the PARP inhibitor is olaparib.

45. The method of any one of claims 26 to 41, wherein the GPX4 inhibitor is RSL3, and the PARP inhibitor is niraparib.

46. The method of any one of claims 1 to 45, further comprising
a. administering chemotherapy;
b. performing surgery;
c. administering radiation therapy;
d. administering targeted therapy; or
e. any combination thereof.

47. The method of any one of claims 1 to 46, wherein the administering reduces the cancer burden.

48. A pharmaceutical composition comprising a GPX4 inhibitor and a PARP inhibitor for use in treating a cancer cell.

49. The composition of claim 48, wherein the GPX4 inhibitor is selected from the group consisting of RSL3, ML162, ML210, JKE-1674, withaferin A, and a combination thereof.

50. The composition of claim 48 or 49, wherein the PARP inhibitor is selected from the group consisting of olaparib, niraparib, veliparib, talazoparib, rucaparib, fluzoparib, and a combination thereof.

51. The composition of any one of claims 48 to 50, wherein the GPX4 inhibitor is JKE-1674, and the PARP inhibitor is olaparib.

52. The composition of any one of claims 48 to 50, wherein the GPX4 inhibitor is withaferin A, and the PARP inhibitor is olaparib.

53. The composition of any one of claims 48 to 50, wherein the GPX4 inhibitor is RSL3, and the PARP inhibitor is olaparib.

54. The composition of any one of claims 48 to 50, wherein the GPX4 inhibitor is RSL3, and the PARP inhibitor is niraparib.

55. The composition of any one of claims 48 to 54, further comprising at least one pharmaceutically acceptable excipient.

56. The composition of claim 55, wherein at least one pharmaceutically acceptable excipient is a pharmaceutically acceptable carrier.

57. The method of any one of claims 1 to 47 or the composition of any one of claims 48 to 56, wherein the cancer is PARP inhibitor resistant.

58. The method of any one of claims 1 to 47 or the composition of any one of claims 48 to 56, wherein the cancer PARP inhibitor sensitive.

59. A method of treating a PARP inhibitor-resistant cancer in a subject in need thereof, comprising administering an effective amount of a PARP inhibitor and GPX4 inhibitor to the subject, wherein the PARP inhibitor-resistant cancer is BRCA1 deficient.

60. The method of claim 59, wherein the BRCA1 deficiency is a BRCA1 mutation.

61. The method of any one of claim 59 or 60, wherein the subject does not have a BRCA2 deficiency.

62. The method of any one of claims 59 to 61, wherein the subject does not have a BRCA2 mutation.

63. The method of any one of claims 59 to 62, wherein the GPX4 inhibitor is selected from the group consisting of RSL3, ML162, ML210, JKE-1674, withaferin A, and a combination thereof.

64. The method of any one of claims 59 to 63, wherein the PARP inhibitor is selected from the group consisting of olaparib, niraparib, veliparib, talazoparib, rucaparib, fluzoparib, and a combination thereof.

65. The method of any one of claims 59 to 64, wherein the GPX4 inhibitor is JKE-1674, and the PARP inhibitor is olaparib.

66. The method of any one of claims 59 to 64, wherein the GPX4 inhibitor is withaferin A, and the PARP inhibitor is olaparib.

67. The method of any one of claims 59 to 64, wherein the GPX4 inhibitor is RSL3, and the PARP inhibitor is olaparib.

68. The method of any one of claims 59 to 64, wherein the GPX4 inhibitor is RSL3, and the PARP inhibitor is niraparib.

69. The method of any one of claims 59 to 68, wherein the PARP inhibitor-resistant cancer is a tumor.

70. The method of claim 69, wherein the tumor is a carcinoma.

71. The method of any one of claims 59 to 70, wherein the PARP inhibitor-resistant cancer is selected from the group consisting of breast cancer, ovarian cancer, colon cancer, pancreatic cancer, and prostate cancer.

72. The method of any one of claims 59 to 71, wherein the PARP inhibitor-resistant cancer is breast cancer.


SEQUENCE LISTING (39 sequences)

SEQ ID NO: 1 - RNA, 25 nt, synthetic construct
  source 1..25: mol_type = other RNA; note = Control-sgRNA-F
caccggcactaccagagctaactca

SEQ ID NO: 2 - RNA, 25 nt, synthetic construct
  source 1..25: mol_type = other RNA; note = Control-sgRNA-R
aaactgagttagctctggtagtgcc

SEQ ID NO: 3 - RNA, 25 nt, synthetic construct
  source 1..25: mol_type = other RNA; note = BRCA1-sgRNA#1-F
caccgtggatttcgcaggtcctcaa

SEQ ID NO: 4 - RNA, 25 nt, synthetic construct
  source 1..25: mol_type = other RNA; note = BRCA1-sgRNA#1-R
aaacttgaggacctgcgaaatccac

SEQ ID NO: 5 - RNA, 24 nt, synthetic construct
  source 1..24: mol_type = other RNA; note = BRCA1-sgRNA#2-F
caccgatgtttccgtcaaatcgtg

SEQ ID NO: 6 - RNA, 24 nt, synthetic construct
  source 1..24: mol_type = other RNA; note = BRCA1-sgRNA#2-R
aaaccacgatttgacggaaacatc

SEQ ID NO: 7 - RNA, 25 nt, synthetic construct
  source 1..25: mol_type = other RNA; note = BRCA2-sgRNA#1-F
caccggtctacctgaccaatcgatg

SEQ ID NO: 8 - RNA, 25 nt, synthetic construct
  source 1..25: mol_type = other RNA; note = BRCA2-sgRNA#1-R
aaaccatcgattggtcaggtagacc

SEQ ID NO: 9 - RNA, 25 nt, synthetic construct
  source 1..25: mol_type = other RNA; note = BRCA2-sgRNA#2-F
caccggaatgccccatcgattggtc

SEQ ID NO: 10 - RNA, 25 nt, synthetic construct
  source 1..25: mol_type = other RNA; note = BRCA2-sgRNA#2-R
aaacgaccaatcgatggggcattcc

SEQ ID NO: 11 - RNA, 25 nt, synthetic construct
  source 1..25: mol_type = other RNA; note = VDAC3-sgRNA#1-F
caccggccagcaagccacccttcga

SEQ ID NO: 12 - RNA, 25 nt, synthetic construct
  source 1..25: mol_type = other RNA; note = VDAC3-sgRNA#1-R
aaactcgaagggtggcttgctggcc

SEQ ID NO: 13 - RNA, 25 nt, synthetic construct
  source 1..25: mol_type = other RNA; note = VDAC3-sgRNA#2-F
caccgctgtgttggccttcgaaggg

SEQ ID NO: 14 - RNA, 25 nt, synthetic construct
  source 1..25: mol_type = other RNA; note = VDAC3-sgRNA#2-R
aaaccccttcgaaggccaacacagc

SEQ ID NO: 15 - RNA, 25 nt, synthetic construct
  source 1..25: mol_type = other RNA; note = GPX4-sgRNA-F
caccgggtgaagcgctacggaccca

SEQ ID NO: 16 - RNA, 25 nt, synthetic construct
  source 1..25: mol_type = other RNA; note = GPX4-sgRNA-R
aaactgggtccgtagcgcttcaccc

SEQ ID NO: 17 - RNA, 25 nt, synthetic construct
  source 1..25: mol_type = other RNA; note = NCOA4-sgRNA-F
caccgaactttcaagatgtaaccgt

SEQ ID NO: 18 - RNA, 25 nt, synthetic construct
  source 1..25: mol_type = other RNA; note = NCOA4-sgRNA-R
aaacacggttacatcttgaaagttc

SEQ ID NO: 19 - RNA, 20 nt, synthetic construct
  source 1..20: mol_type = other RNA; note = 53BP1-sgRNA
ggaaagactgtcctgaacaa

SEQ ID NO: 20 - RNA, 25 nt, synthetic construct
  source 1..25: mol_type = other RNA; note = ECAD-sgRNA#1-F
caccggcaatgcgttctctatccag

SEQ ID NO: 21 - RNA, 25 nt, synthetic construct
  source 1..25: mol_type = other RNA; note = ECAD-sgRNA#1-R
aaacctggatagagaacgcattgcc

SEQ ID NO: 22 - RNA, 25 nt, synthetic construct
  source 1..25: mol_type = other RNA; note = ECAD-sgRNA#2-F
caccggctgcggctccaagggccca

SEQ ID NO: 23 - RNA, 25 nt, synthetic construct
  source 1..25: mol_type = other RNA; note = ECAD-sgRNA#2-R
aaactgggcccttggagccgcagcc

SEQ ID NO: 24 - RNA, 17 nt, synthetic construct
  source 1..17: mol_type = other RNA; note = VDAC3-F
tctggaccaaccatcta

SEQ ID NO: 25 - RNA, 17 nt, synthetic construct
  source 1..17: mol_type = other RNA; note = VDAC3-R
aggctggcattatttac

SEQ ID NO: 26 - RNA, 22 nt, synthetic construct
  source 1..22: mol_type = other RNA; note = GPX4-F
acaagaacggctgcgtggtgaa

SEQ ID NO: 27 - RNA, 22 nt, synthetic construct
  source 1..22: mol_type = other RNA; note = GPX4-R
gccacacacttgtggagctaga

SEQ ID NO: 28 - RNA, 23 nt, synthetic construct
  source 1..23: mol_type = other RNA; note = NCOA4-F
gcttgctattggtggagttctcc

SEQ ID NO: 29 - RNA, 22 nt, synthetic construct
  source 1..22: mol_type = other RNA; note = NCOA4-R
gccatacctcacggcttctaag

SEQ ID NO: 30 - RNA, 17 nt, synthetic construct
  source 1..17: mol_type = other RNA; note = Beta actin-F
cggaaccgctcattgcc

SEQ ID NO: 31 - RNA, 20 nt, synthetic construct
  source 1..20: mol_type = other RNA; note = Beta actin-R
acccacactgtgcccatcta

SEQ ID NO: 32 - RNA, 25 nt, synthetic construct
  source 1..25: mol_type = other RNA; note = VDAC3-ChIP-F
gctccagtgagccaagatcgcgcca

SEQ ID NO: 33 - RNA, 26 nt, synthetic construct
  source 1..26: mol_type = other RNA; note = VDAC3-ChIP-R
gttgctagttaactgttgccatgggt

SEQ ID NO: 34 - RNA, 23 nt, synthetic construct
  source 1..23: mol_type = other RNA; note = GPX4-BS1-ChIP-F
gtttcctgagaagtcagacaatg

SEQ ID NO: 35 - RNA, 25 nt, synthetic construct
  source 1..25: mol_type = other RNA; note = GPX4-BS1-ChIP-R
ccagatgcagcacagaggcgtcatg

SEQ ID NO: 36 - RNA, 25 nt, synthetic construct
  source 1..25: mol_type = other RNA; note = GPX4-BS2-ChIP-F
gtgtttctgttgccacatgtccaag

SEQ ID NO: 37 - RNA, 23 nt, synthetic construct
  source 1..23: mol_type = other RNA; note = GPX4-BS2-ChIP-R
gctcccacccccaggggcagctg

SEQ ID NO: 38 - RNA, 21 nt, synthetic construct
  source 1..21: mol_type = other RNA; note = GPX4-BS3-ChIP-F
gagggcgtggccgtgggaaag

SEQ ID NO: 39 - RNA, 21 nt, synthetic construct
  source 1..21: mol_type = other RNA; note = GPX4-BS3-ChIP-R
cggcgctagctcaccatggtc